(19) United States
(12) Patent Application Publication
(10) Pub. No.: US 2013/0052203 A1
(43) Pub. Date: Feb. 28, 2013

(54) IN VIVO SCREENING MODELS FOR TREATMENT OF QC-RELATED DISORDERS

(71) Applicants: Sigrid Graubner, Munchen (DE); Wolfgang Jagla, Windach (DE); Stephan Schilling, Halle/Saale (DE); Holger Cynis, Halle/Saale (DE); Hans-Ulrich Demuth, Halle/Saale (DE); Torsten Hoffmann, Halle/Saale (DE); Michael Wermann, Halle (DE); Katrin Schulz, Wansleben am See (DE); Christoph Baeuscher, Unterhaching (DE); Stefanie Kohlmann, Unterhaching (DE)

(72) Inventors: Sigrid Graubner, Munchen (DE); Wolfgang Jagla, Windach (DE); Stephan Schilling, Halle/Saale (DE); Holger Cynis, Halle/Saale (DE); Hans-Ulrich Demuth, Halle/Saale (DE); Torsten Hoffmann, Halle/Saale (DE); Michael Wermann, Halle (DE); Katrin Schulz, Wansleben am See (DE); Christoph Baeuscher, Unterhaching (DE); Stefanie Kohlmann, Unterhaching (DE)

(73) Assignee: PROBIODRUG AG, Halle/Saale (DE)

(21) Appl. No.: 13/571,947

(22) Filed: Aug. 10, 2012

Related U.S. Application Data
(60) Provisional application No. 61/522,900, filed on Aug. 12, 2011.

(51) Int. Cl. A01K 67/027; A61K 49/00; C12N 5/071; C07K 16/40; A61K 39/395; A61P 11/00; A61P 25/18; A61P 35/00; A61P 19/02; A61P 35/04; A61P 9/10; A61P 1/18; A01K 67/02; A61P 25/28

(52) U.S. Cl. 424/158.1; 800/9; 800/22; 800/3; 435/354; 530/389.8

(57) ABSTRACT

A double transgenic non-human animal, in particular a transgenic mouse encoding Qpct proteins, which have been implicated in Qpct-related diseases, and Amyloid Precursor Protein (APP). Also provided are cells and cell lines comprising transgenes encoding for Qpct and APP. Also provided are methods and compositions for evaluating agents that affect Qpct, for use in compositions for the treatment of Qpct-related diseases.

### CROSS-REFERENCE TO RELATED APPLICATIONS

This application claims priority from U.S. Provisional Application Ser. No. 61/522,900, filed on Aug. 12, 2011, which is incorporated herein by reference in its entirety.

### MATERIAL INCORPORATED BY REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present invention relates generally to transgenic animals as well as methods and compositions for screening and treating QC-related disorders, especially Alzheimer's disorder.

### BACKGROUND OF THE INVENTION

Glutaminyl cyclase (QC, EC 2.3.2.5; Qpct; glutaminyl peptide cyclotransferase) catalyzes the intramolecular cyclization of N-terminal glutamine residues into pyroglutamic acid (5-oxo-proline, pGlu*) under liberation of ammonia and the intramolecular cyclization of N-terminal glutamate residues into pyroglutamic acid under liberation of water.

A QC was first isolated by Messer from the Latex of the tropical plant Carica papaya in 1963 (Messer, M. 1963 Nature 4874, 1299). 24 years later, a corresponding enzymatic activity was discovered in animal pituitary (Busby, W. H. J. et al. 1987 J Biol Chem 262, 8532-8536; Fischer, W. H. and Spiess, J. 1987 Proc Natl Acad Sci U S A 84, 3628-3632). For the mammalian QC, the conversion of Gln into pGlu by QC could be shown for the precursors of TRH and GnRH (Busby, W. H. J. et al. 1987 J Biol Chem 262, 8532-8536; Fischer, W. H. and Spiess, J. 1987 Proc Natl Acad Sci U S A 84, 3628-3632). In addition, initial localization experiments of QC revealed a co-localization with its putative products of catalysis in bovine pituitary, further improving the suggested function in peptide hormone synthesis (Bockers, T. M. et al. 1995 J Neuroendocrinol 7, 445-453). In contrast, the physiological function of the plant QC is less clear. In the case of the enzyme from C. papaya, a role in the plant defense against pathogenic microorganisms was suggested (El Moussaoui, A. et al. 2001 Cell Mol Life Sci 58, 556-570). Putative QCs from other plants were identified by sequence comparisons recently (Dahl, S. W. et al. 2000 Protein Expr Purif 20, 27-36). The physiological function of these enzymes, however, is still ambiguous.

The QCs known from plants and animals show a strict specificity for L-glutamine in the N-terminal position of the substrates and their kinetic behavior was found to obey the Michaelis-Menten equation (Pohl, T. et al. 1991 Proc Natl Acad Sci U S A 88, 10059-10063; Consalvo, A. P. et al. 1988 Anal Biochem 175, 131-138; Gololobov, M. Y. et al. 1996 Biol Chem Hoppe Seyler 377, 395-398). A comparison of the primary structures of the QCs from C. papaya and that of the highly conserved QC from mammals, however, did not reveal any sequence homology (Dahl, S. W. et al. 2000 Protein Expr Purif 20, 27-36). Whereas the plant QCs appear to belong to a new enzyme family (Dahl, S. W. et al. 2000 Protein Expr Purif 20, 27-36), the mammalian QCs were found to have a pronounced sequence homology to bacterial aminopeptidases (Bateman, R. C. et al. 2001 Biochemistry 40, 11246-11250), leading to the conclusion that the QCs from plants and animals have different evolutionary origins.

EP 02 011 349.4 discloses polynucleotides encoding insect glutaminyl cyclase, as well as polypeptides encoded thereby. This application further provides host cells comprising expression vectors comprising polynucleotides of the invention. Isolated polypeptides and host cells comprising insect QC are useful in methods of screening for agents that reduce glutaminyl cyclase activity. Such agents are described as useful as pesticides.

The subject matter of the present invention is particularly useful in the field of QC-related diseases, one example of those being Alzheimer's Disease. Alzheimer's disease (AD) is characterized by abnormal accumulation of extracellular amyloidotic plaques closely associated with dystrophic neurones, reactive astrocytes and microglia (Terry, R. D. and Katzman, R. 1983 Ann Neurol 14, 497-506; Glenner, G. G. and Wong, C. W. 1984 Biochem Biophys Res Comm 120, 885-890; Intagaki, S. et al. 1989 J Neuroimmunol 24, 173-182; Funato, H. et al. 1998 Am J Pathol 152, 983-992; Selkoe, D. J. 2001 Physiol Rev 81, 741-766). Amyloid-beta (abbreviated as Aβ) peptides are the primary components of senile plaques and are considered to be directly involved in the pathogenesis and progression of AD, a hypothesis supported by genetic studies (Glenner, G. G. and Wong, C. W. 1984 Biochem Biophys Res Comm 120, 885-890; Borchelt, D. R. et al. 1996 Neuron 17, 1005-1013; Lemere, C. A. et al. 1996 Nat Med 2, 1146-1150; Mann, D. M. and Iwatsubo, T. 1996 Neurodegeneration 5, 115-120; Citron, M. et al. 1997 Nat Med 3, 67-72; Selkoe, D. J. 2001 Physiol Rev 81, 741-766). Aβ is generated by proteolytic processing of the β-amyloid precursor protein (APP) (Kang, J. et al. 1987 Nature 325, 733-736; Selkoe, D. J. 1998 Trends Cell Biol 8, 447-453), which is sequentially cleaved by β-secretase at the N-terminus and by γ-secretase at the C-terminus of Aβ (Haass, C. and Selkoe, D. J. 1993 Cell 75, 1039-1042; Simons, M. et al. 1996 J Neurosci 16 899-908). In addition to the dominant Aβ peptides starting with L-Asp at the N-terminus (Aβ1-42/40), a great heterogeneity of N-terminally truncated forms occurs in senile plaques. Such shortened peptides are reported to be more neurotoxic in vitro and to aggregate more rapidly than the full-length isoforms (Pike, C. J. et al. 1995 J Biol Chem 270, 23895-23898). N-truncated peptides are known to be overproduced in early onset familial AD (FAD) subjects (Saido, T. C. et al. 1995 Neuron 14, 457-466; Russo, C, et al. 2000 Nature 405, 531-532), to appear early and to increase with age in Down's syndrome (DS) brains (Russo, C. et al. 1997 FEBS Lett 409, 411-416, Russo, C. et al. 2001 Neurobiol Dis 8, 173-180; Tekirian, T. L. et al. 1998 J Neuropathol Exp Neurol 57, 76-94). Finally, their amount reflects the progressive severity of the disease (Russo, C. et al. 1997 FEBS Lett 409, 411-416; Guntert, A. et al. 2006 Neuroscience 143, 461-475).

Additional post-translational processes may further modify the N-terminus by isomerization or racemization of the aspartate at position 1 and 7 and by cyclization of glutamate at residues 3 and 11. Pyroglutamate-containing isoforms at position 3 (pGluAβ3-40/42/43) represent the prominent forms—approximately 50% of the total Aβ amount—of the N-truncated species in senile plaques (Mori, H. et al. 1992 J Biol Chem 267, 17082-17086, Saido, T. C. et al. 1995 Neuron 14, 457-466; Russo, C. et al. 1997 FEBS Lett 409, 411-416; Tekirian, T. L. et al. 1998 J Neuropathol Exp Neurol 57, 76-94; Geddes, J. W. et al. 1999 Neurobiol Aging 20, 75-79; Harigaya, Y. et al. 2000 Biochem Biophys Res Commun 276, 422-427) and they are also present in pre-amyloid lesions (Lalowski, M. et al. 1996 J Biol Chem 271, 33623-33631). The accumulation of the pGluAβ3-40/42/43 peptides is likely due to the structural modification that enhances aggregation and confers resistance to most amino-peptidases (Saido, T. C. et al. 1995 Neuron 14, 457-466; Tekirian, T. L. et al. 1999 J Neurochem 73, 1584-1589). This evidence provides clues for a pivotal role of AβN3(pE) peptides in AD pathogenesis. However, relatively little is known about their neurotoxicity and aggregation properties (He, W. and Barrow, C. J. 1999 Biochemistry 38, 10871-10877; Tekirian, T. L. et al. 1999 J Neurochem 73, 1584-1589). Moreover, the action of these isoforms on glial cells and the glial response to these peptides are completely unknown, although activated glia is strictly associated with senile plaques and might actively contribute to the accumulation of amyloid deposits.

In recent studies the toxicity, aggregation properties and catabolism of Aβ1-42, Aβ1-40, pGluAβ3-42, pGluAβ3-40, pGluAβ11-42 and pGluAβ11-40 peptides were investigated in neuronal and glial cell cultures, and it was shown that pyroglutamate modification exacerbates the toxic properties of Aβ-peptides and also inhibits their degradation by cultured astrocytes. Shirotani et al. investigated the generation of pGluAβ(3-x) peptides in primary cortical neurons infected by Sindbis virus in vitro. They constructed amyloid precursor protein complementary DNAs, which encoded a potential precursor for pGluAβ by amino acid substitution and deletion. For one artificial precursor starting with a N-terminal glutamine residue instead of glutamate in the natural precursor, a spontaneous conversion or an enzymatic conversion by glutaminyl cyclase to pyroglutamate was suggested. The cyclization mechanism of N-terminal glutamate at position 3 in the natural precursor of pGluAβ(3-x) was neither determined in vitro, in situ nor in vivo (Shirotani, K. et al. 2002 NeuroSci Lett 327, 25-28).

WO 2008/087197 describes an in vivo screening model for the treatment of Alzheimer's disease and other QC-related disorders which comprises a transgenic mouse encoding QC. Accordingly, it is an object of the invention to provide a transgenic animal, which overexpresses both APP and QC. It is another object of the invention to provide DNA constructs encoding APP and QC. It is an additional object of the invention to provide DNA constructs encoding APP linked to a promoter and QC linked to a promoter. It is an additional object of the invention to provide a non-human transgenic animal model system to study the in vivo and in vitro regulation and effects of APP and QC in specific tissue types.

### SUMMARY OF THE INVENTION

The invention comprises methods and compositions for non-human transgenic, in particular mammal, models for QC-related diseases. Specifically, the present invention comprises non-human transgenic animal models that overexpress APP and QC.

The present invention further comprises compositions and methods for screening for biologically active agents that modulate QC-related diseases including, but not limited to, Mild Cognitive Impairment (MCI), Alzheimer's Disease (AD), cerebral amyloid angiopathy, Lewy body dementia, neurodegeneration in Down Syndrome, hereditary cerebral hemorrhage with amyloidosis (Dutch type), Familial Danish Dementia, Familial British Dementia, ulcer disease and gastric cancer with or w/o Helicobacter pylori infections, pathogenic psychotic conditions, schizophrenia, infertility, neoplasia, inflammatory host responses, cancer, psoriasis, rheumatoid arthritis, atherosclerosis, restenosis, lung fibrosis, liver fibrosis, renal fibrosis, Acquired Immune Deficiency Syndrome, graft rejection, Chorea Huntington (HD), impaired humoral and cell-mediated immune responses, leukocyte adhesion and migration processes in the endothelium, impaired food intake, sleep-wakefulness, impaired homeostatic regulation of energy metabolism, impaired autonomic function, impaired hormonal balance and impaired regulation of body fluids and the Guam Parkinson-Dementia complex. Another aspect of the present invention comprises methods and compositions for screening for QC inhibitors.

Further, by administration of effectors of QC activity to a mammal it can be possible to stimulate gastrointestinal tract cell proliferation, preferably proliferation of gastric mucosal cells, epithelial cells, acute acid secretion and the differentiation of acid producing parietal cells and histamine-secreting enterochromaffin-like cells.

Furthermore, by administration of effectors of QC activity to a mammal it can be possible to suppress the proliferation of myeloid progenitor cells.

In addition, administration of QC inhibitors can lead to suppression of male fertility.

The present invention provides pharmaceutical compositions for parenteral, enteral or oral administration, comprising at least one effector of QC optionally in combination with customary carriers and/or excipients.

Additionally, the present invention comprises methods and compositions for the treatment and/or prevention of QC-related diseases, particularly methods and compositions that inhibit or promote QC.

It was shown by inhibition studies that human and murine QC are metal-dependent transferases. QC apoenzyme could be reactivated most efficiently by zinc ions, and the metal-binding motif of zinc-dependent aminopeptidases is also present in human QC. Compounds interacting with the active-site bound metal are potent inhibitors.

Unexpectedly, it was shown that recombinant human QC as well as QC-activity from brain extracts catalyze both, the N-terminal glutaminyl as well as glutamate cyclization. Most striking is the finding, that QC-catalyzed Glu-conversion is favored around pH 6.0 while Gln-conversion to pGlu-derivatives occurs with a pH-optimum of around 8.0. Since the formation of pGlu-Aβ-related peptides can be suppressed by inhibition of recombinant human QC and QC-activity from pig pituitary extracts, the enzyme QC is a target in drug development for treatment of e.g. Alzheimer's disease.

Other objects and features will be in part apparent and in part pointed out hereinafter.

### BRIEF DESCRIPTION OF THE FIGURES

Those of skill in the art will understand that the drawings, described below, are for illustrative purposes only. The drawings are not intended to limit the scope of the present teachings in any way.

Further understanding of these and other aspects of the present invention will be gained by reference to the figures, which represent the following:

FIG. 1: Transgenic Plasmid pUC18-mThy1-hAPP695wt used to generate APP695 transgenic mice.

FIG. 2: 1% Agarose/1xTBE gel which shows 8931 by fragment for microinjection to generate APP695 transgenic mice.

FIG. 3: Single construct in head-to-tail PCR for identification of APP695 transgenic founders.

FIG. 4: Incomplete constructs in head-to-tail PCR for identification of APP695 transgenic founders.

FIG. 5: Multiple constructs in head-to-tail PCR for identification of APP695 transgenic founders.

FIG. 6: mRNA expression level of different mTHy1-hAPP695-wt transgenic mouse lines in the cortex.

FIG. 7: mRNA expression level of different mTHy1-hAPP695-wt transgenic mouse lines in the hippocampus.

FIG. 8: mRNA expression level of different mTHy1-hAPP695-wt transgenic mouse lines in the spinal cord.

FIG. 9: Transgenic Plasmid pUC18-mThy1-hQC used to generate QC transgenic mice.

FIG. 10: 0.8% Agarose/1xTBE gel which shows control cut of transgenic plasmid for microinjection to generate QC transgenic mice.

FIG. 11: Multiple constructs in head-to-tail PCR for identification of QC transgenic founders.

FIG. 12: mRNA expression level of different mTHy1-hQC transgenic mouse founder lines in the cortex (Co), hippocampus (Hi) and spinal cord (SC) of Fo#37, Fo#43 and Fo#53.

FIG. 13: APPwt transgenic expression cassette.

FIG. 14: hQC transgenic expression cassette.

FIG. 15: Genotyping assay for APPwt

FIG. 16: Gel electrophoresis for the detection of the APPwt transgene by PCR.

FIG. 17: (A): Genomic map of the APPwt-35 transgene; (B): Gel electrophoresis APPwt PCR.

FIG. 18: Genotyping assay for hQC.

FIG. 19: (A): Genomic map of the hQC-43-35 transgene; (B): Gel electrophoresis hQC-43.

FIG. 20: (A): Genomic map of the hQC-53-35 transgene; (B): Gel electrophoresis hQC-53.

FIG. 21: Genotyping assay for hQC.

FIG. 22: Cloning strategy of integration site.

FIG. 23: ELISA human APP detection in several transgenic hAPP mouse lines.

FIG. 24: Western analysis of right cerebrum (A) and cerebellum (B) of transgenic hAPP mouse derived from different founder lines (−31, 35, −37, −38 and −40).

FIG. 25: Transgene expression levels in hQC lines.

FIG. 26: hC43 transgene expression rates in 2 month old animals in comparison to 24 month old animals.

FIG. 27: QC-activity in brain samples and plasma from transgenic animals, which express human QC neuron-specifically.

FIG. 28: Localization of overexpressed Amyloid. IHC with anti-x-4xAβ/6E10 (Covance; mouse monoclonal, 1:30000) in APP-Aw35-wt and APP-Aw35-hom on coronal brain sections showing intra- and extracellular immunoreactivity to amyloid (APP or Abeta) in cortex at the age of 4 months in APP-Aw35-hom (C, D).

FIG. 29: Localization of overexpressed Amyloid Precursor Protein (APP). IHC with anti-APP (Synaptic Systems; C-terminal, rabbit polyclonal, 1:100000) in APP-Aw35-wt and APP-Aw35-hom on coronal brain sections showing intraneuronal immunoreactivity in the cortex at the age of 4 months in APP-Aw35-hom (C, D).

FIG. 30: Immunhistochemical staining of coronal brain sections (striatum) with human QC-specific antibody.

FIG. 31: Weight analysis of one APP-wt-31 male set revealed significantly lower weight in transgene animals compared to wildtype littermates (mean+SEM) at 2 different stages of life (3 months: t-test p<0.05; 6 months: t-test p<0.001).

FIG. 32: Vector maps of APP and hQC.

FIG. 33: Progress in plaque pathology between the age of 18 & 26 months. Immunohistochemistry (IHC) with anti-N3pE-Aβ (Synaptic Systems; rabbit polyclonal, 1:100000) in APPhom/HQChet on coronal brain sections at the age of 18 months (A) and 26 months (B) showing appearance of rare plaques in the subiculum in the younger animals (A, A′), and a clear plaque pathology in cortical and hippocampal regions in the older animals (B). Blue hematoxylin counterstain.

FIG. 34: Plaque pathology. IHC with anti-N3pE-Aβ (Synaptic Systems; rabbit polyclonal, 1:100000) and anti-APP (Synaptic systems; C-terminal, rabbit polyclonal, 1:100000) in APPhom/HQChet on parallel coronal brain sections showing advanced plaque pathology in cortical and hippocampal regions at the age of 26 months. Blue hematoxylin counterstain.

FIG. 35: N-terminally truncated Aβ in plaques. IHC with anti-N3pE-Aβ (Synaptic Systems; rabbit polyclonal, 1:100000) in APPwt/HQChet and APPhom/HQChet on coronal brain sections showing plaque pathology in cortical and hippocampal regions at the age of 24 months in (hom/het). No counterstain.

FIG. 36: Localization of overexpressed Amyloid Precursor Protein (APP). IHC with anti-APP (Synaptic Systems; C-terminal, rabbit polyclonal, 1:100000) in APPwt/HQChet and APPhom/HQChet on coronal brain sections showing intraneuronal immunoreactivity and plaque pathology in cortical and hippocampal regions at the age of 24 months in (hom/het).

FIG. 37: Plaque pathology. IHC with anti-x-4xAβ/6E10 (Covance; mouse monoclonal, 1:30000) in APPwt/HQChet and APPhom/HQChet on coronal brain sections showing strong specific intra- and extracellular immunoreactivity as well as plaque pathology in cortical and hippocampal regions at the age of 24 months in (hom/het).

FIG. 38: Plaque pathology. IHC with anti-N1-4x-Aβ (IBL; N-terminal-specific; rabbit polyclonal, 1:30000) in APPwt/HQChet and APPhom/HQChet on coronal brain sections showing plaque pathology in cortical and hippocampal regions at the age of 24 months in (hom/het). No counterstain.

FIG. 39: N-terminally truncated Aβ in plaques. IHC with anti-N11pE Aβ (Probiodrug; mouse monoclonal, 1:30000) in APPwt/HQChet and APPhom/HQChet on coronal brain sections showing plaque pathology in cortical and hippocampal regions at the age of 24 months in (hom/het). Unspecific background staining is comparable in (hom/het) & (wt/het).

FIG. 40: Diffuse plaques and dense core plaques. IHC with anti-N3pE-Abeta (Synaptic Systems; rabbit polyclonal, 1:100000) (A, B) and anti-APP (Synaptic systems; c-terminal, rabbit polyclonal, 1:100000) (C, D) in APPwt/HQChet and APPhom/HQChet on coronal brain sections illustrating plaque composition in cortex at the age of 26 months in (hom/het). Blue hematoxylin counterstain.

FIG. 41: Neuroinflammation associated with dense core plaques. IHC in APPhom/HQChet coronal brain sections with anti-GFAP (DAKO, rabbit polyclonal, 1:10000) (A, B) showing activated astrocytes around and between Congo Red-positive plaques in cortex at the age of 26 months. Blue hematoxylin counterstain. The same Congo Red positive plaques are visualized separately using a fluorescence microscope (C, D).

FIG. 42: Analyzing the period of immobility during a 6-minutes trial in the tail suspension assay revealed significantly decreased values (i.e. higher activity levels) in HOM/tg females compared to WT/tg littermates (A: set 1, t-test p=0.0001; B: set 2, t-test p=0.0205); mean and individual data points.

FIG. 43: Assessment of nociception using the tail flick test disclosed a statistically significant prolongation of tail withdrawal latency of HOM/tg APP35/hQC63 females in set 1 (A: t-test p=0.0144), but not in set 2 (B: t-test p=0.2025); mean and individual averages of 3 trials.

FIG. 44: Best trial analysis of t-turn in the pole test delivered significantly increased t-turn latencies in HOM/tg animals of set 2 (B: t-test p=0.0081) and by trend of set 1 (A: t-test p=0.0612) when compared to WT/tg littermates; mean and individual data points.

### DETAILED DESCRIPTION OF THE INVENTION

According to a first aspect of the invention, there is provided a transgenic non-human animal for overexpressing APP and QC.

The advantage of the present invention is the provision of a non-human animal which is able to effectively mimic the human situation in an Alzheimer's model. For example, the ability to overexpress human APP and human QC in a single model allows the entire APP cleavage pathway to be operable resulting in the formation of neurotoxic forms of Aβ, preferably N-terminally truncated forms of Aβ, such as Aβ(3-x) and/or Aβ(11-x), being rapidly catalyzed by human QC to N-terminally truncated forms of Aβ that contain a pyroglutamate (pGlu) residue at the N-terminus, such as pGlu-Aβ(3-x) and/or pGlu-Aβ(11-x), wherein x is an integer between 35 and 45. Preferably, x is an integer selected from 37, 38, 39, 40, 41, 42 and 43. More preferably, x is an integer selected from 39, 40, 41, 42 and 43. Most preferably, x is an integer selected from 40, 42 and 43.

In one embodiment, the transgenic non-human animal comprises cells containing one or more DNA transgenes encoding human APP and human QC.

In one embodiment, the human QC comprises the amino acid sequence of SEQ ID NO: 1 or an amino acid sequence having at least 75% sequence identity to the amino acid sequence of SEQ ID NO: 1 or a fragment or derivative of the amino acid sequence of SEQ ID NO: 1.

In one embodiment, the human APP comprises human APP695 or human APP770. In a further embodiment, the human APP comprises human APP695 as defined by the amino acid sequence of SEQ ID NO: 2 or an amino acid sequence having at least 75% sequence identity to the amino acid sequence of SEQ ID NO: 2 or a fragment or derivative of the amino acid sequence of SEQ ID NO: 2. In an alternative embodiment, the human APP comprises human APP770 as defined by the amino acid sequence of SEQ ID NO: 3 or an amino acid sequence having at least 75% sequence identity to the amino acid sequence of SEQ ID NO: 3 or a fragment or derivative of the amino acid sequence of SEQ ID NO: 3.

When amino acids, peptides or polypeptides are referred to herein, it will be appreciated that the amino acid residue will be represented by a one-letter or a three-letter designation, corresponding to the trivial name of the amino acid, in accordance with the following conventional list:

| Amino Acid | One-Letter Symbol | Three-Letter Symbol |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

In one embodiment, the human QC has an amino acid sequence having at least 80% sequence identity to the amino acid sequence of SEQ ID NO: 1, such as a sequence identity selected from any one of 85%, 86%, 87%, 88%, 89%, 90%, 91%, 92%, 93%, 94%, 95%, 96%, 97%, 98% or 99% sequence identity to the amino acid sequence of SEQ ID NO: 1. In a particular embodiment, the human QC consists of the amino acid sequence of SEQ ID NO: 1.

In one embodiment, the human APP695 has an amino acid sequence having at least 80% sequence identity to the amino acid sequence of SEQ ID NO: 2, such as a sequence identity selected from any one of 85%, 86%, 87%, 88%, 89%, 90%, 91%, 92%, 93%, 94%, 95%, 96%, 97%, 98% or 99% sequence identity to the amino acid sequence of SEQ ID NO: 2. In a particular embodiment, the human APP695 consists of the amino acid sequence of SEQ ID NO: 2.

In one embodiment, the human APP770 has an amino acid sequence having at least 80% sequence identity to the amino acid sequence of SEQ ID NO: 3, such as a sequence identity selected from any one of 85%, 86%, 87%, 88%, 89%, 90%, 91%, 92%, 93%, 94%, 95%, 96%, 97%, 98% or 99% sequence identity to the amino acid sequence of SEQ ID NO: 3. In a particular embodiment, the human APP770 consists of the amino acid sequence of SEQ ID NO: 3.

In one embodiment, the human QC comprises a fragment or derivative of the amino acid sequence of SEQ ID NO: 1. It will be appreciated that when the human QC comprises a fragment of the amino acid sequence of SEQ ID NO: 1 it will be required to be a fragment which retains some or all of the function of the full-length QC amino acid sequence described in SEQ ID NO: 1. References herein to “derivative of the amino acid sequence of SEQ ID NO: 1” include modifications of the amino acid sequence of SEQ ID NO: 1.

In one embodiment, the human APP695 comprises a fragment or derivative of the amino acid sequence of SEQ ID NO: 2. It will be appreciated that when the human APP695 comprises a fragment of the amino acid sequence of SEQ ID NO: 2 it will be required to be a fragment which retains some or all of the function of the full-length APP amino acid sequence described in SEQ ID NO: 2. References herein to “derivative of the amino acid sequence of SEQ ID NO: 2” include modifications of the amino acid sequence of SEQ ID NO: 2.

In one embodiment, the human APP770 comprises a fragment or derivative of the amino acid sequence of SEQ ID NO: 3. It will be appreciated that when the human APP770 comprises a fragment of the amino acid sequence of SEQ ID NO: 3 it will be required to be a fragment which retains some or all of the function of the full-length APP amino acid sequence described in SEQ ID NO: 3. References herein to “derivative of the amino acid sequence of SEQ ID NO: 3” include modifications of the amino acid sequence of SEQ ID NO: 3.

Individual substitutions, deletions or additions, which alter, add or delete a single amino acid or a small percentage of amino acids (typically less than 10%, more typically less than 5%, and still more typically less than 1%.) A “modification” of the amino acid sequence encompasses conservative substitutions of the amino acid sequence. Conservative substitution tables providing functionally similar amino acids are well known in the art. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (1), Leucine (L), Methionine (M), Valine (V); and
6) Phenylalanine (F), Tyrosine (Y), Tryptophan (W).

Other minor modifications are included within the sequence so long as the polypeptide retains some or all of the structural and/or functional characteristics of the QC polypeptide of SEQ ID NO: 1 or the APP polypeptides of SEQ ID NOS: 2 or 3. Exemplary structural or functional characteristics include sequence identity or substantial similarity, antibody reactivity, the presence of conserved structural domains such as RNA binding domains or acidic domains.

It will be appreciated that references herein to QC refer to glutaminyl peptide cyclotransferase (EC 2.3.2.5.; also known as Qpct, QPCTL or QC-like enzyme) and QC-like enzymes. QC and QC-like enzymes have identical or similar enzymatic activity, further defined as QC activity. In this regard, QC-like enzymes can fundamentally differ in their molecular structure from QC.

The term “QC activity” as used herein is defined as intramolecular cyclization of N-terminal glutamine residues into pyroglutamic acid (pGlu*) or of N-terminal L-homoglutamine or L-β-homoglutamine to a cyclic pyro-homoglutamine derivative under liberation of ammonia. See Schemes 1 and 2.

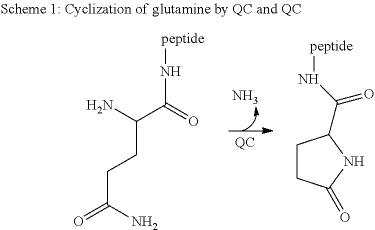

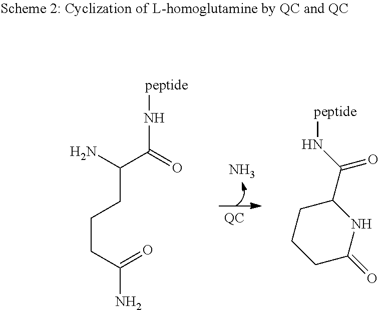

References herein to the term “QC-related disease” or “QC-related disorder refers to all diseases, disorders or conditions that are modulated by QC.

References herein to “APP” refer to amyloid precursor protein. APP is is an integral membrane protein expressed in many tissues and concentrated in the synapses of neurons. APP has been implicated as a regulator of synapse formation, neural plasticity and iron export. APP is best known and most commonly studied as the precursor molecule whose proteolysis generates beta amyloid (An), a 39- to 42-amino acid peptide whose amyloid fibrillar form is the primary component of amyloid plaques found in the brains of Alzheimer's disease patients.

References herein to the term “transgene” include a segment of DNA that has been incorporated into a host genome or is capable of autonomous replication in a host cell and is capable of causing the expression of one or more cellular products. Exemplary transgenes will provide the host cell, or animals developed therefrom, with a novel phenotype relative to the corresponding non-transformed cell or animal.

In one embodiment, the DNA transgene encoding QC comprises the nucleotide sequence of SEQ ID NO: 4 or substantially the same nucleotide sequence of SEQ ID NO: 4.

In one embodiment, the DNA transgene encoding APP695 comprises the nucleotide sequence of SEQ ID NO: 5 or substantially the same nucleotide sequence of SEQ ID NO: 5.

In one embodiment, the DNA transgene encoding APP770 comprises the nucleotide sequence of SEQ ID NO: 6 or substantially the same nucleotide sequence of SEQ ID NO: 6.

The QC polynucleotides comprising the transgene of the present invention include QC cDNA and shall also include modified QC cDNA. The APP polynucleotides comprising the transgene of the present invention include APP cDNA and shall also include modified APP cDNA. As used herein, a “modification” of a nucleic acid can include one or several nucleotide additions, deletions, or substitutions with respect to a reference sequence. A modification of a nucleic acid can include substitutions that do not change the encoded amino acid sequence due to the degeneracy of the genetic code, or which result in a conservative substitution. Such modifications can correspond to variations that are made deliberately, such as the addition of a Poly A tail, or variations which occur as mutations during nucleic acid replication.

References herein to “substantially the same nucleotide sequence” refers to DNA having sufficient identity to the reference polynucleotide, such that it will hybridize to the reference nucleotide under moderately stringent, or higher stringency, hybridization conditions. DNA having “substantially the same nucleotide sequence” as the reference nucleotide sequence, can have an identity ranging from at least 60% to at least 95% with respect to the reference nucleotide sequence.

The phrase “moderately stringent hybridization” refers to conditions that permit a target-nucleic acid to bind a complementary nucleic acid. The hybridized nucleic acids will generally have an identity within a range of at least about 60% to at least about 95%. Moderately stringent conditions are conditions equivalent to hybridization in 50% formamide, 5×Denhart's solution, 5×saline sodium phosphate EDTA buffer (SSPE), 0.2% SDS (Aldrich) at about 42° C., followed by washing in 0.2×SSPE, 0.2% SDS (Aldrich), at about 42° C.

High stringency hybridization refers to conditions that permit hybridization of only those nucleic acid sequences that form stable hybrids in 0.018M NaCl at about 65° C., for example, if a hybrid is not stable in 0.018M NaCl at about 65° C., it will not be stable under high stringency conditions, as contemplated herein. High stringency conditions can be provided, for example, by hybridization in 50% formamide, 5×Denhart's solution, 5×SSPE, 0.2% SDS at about 42° C., followed by washing in 0.1×SSPE, and 0.1% SDS at about 65° C.

Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al. (Current Protocols in Molecular Biology (Supplement 47), John Wiley & Sons, New York (1999)).

In one embodiment, the DNA transgene encoding QC has a nucleotide sequence having at least 75% sequence identity to the nucleotide sequence of SEQ ID NO: 4, such as a sequence identity selected from any one of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleotide sequence of SEQ ID NO: 4. In a particular embodiment, the DNA transgene encoding QC consists of the nucleotide sequence of SEQ ID NO: 4.

In one embodiment, the DNA transgene encoding APP695 has a nucleotide sequence having at least 75% sequence identity to the nucleotide sequence of SEQ ID NO: 5, such as a sequence identity selected from any one of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleotide sequence of SEQ ID NO: 5. In a particular embodiment, the DNA transgene encoding APP695 consists of the nucleotide sequence of SEQ ID NO: 5.

In one embodiment, the DNA transgene encoding APP770 has a nucleotide sequence having at least 75% sequence identity to the nucleotide sequence of SEQ ID NO: 6, such as a sequence identity selected from any one of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleotide sequence of SEQ ID NO: 6. In a particular embodiment, the DNA transgene encoding APP770 consists of the nucleotide sequence of SEQ ID NO: 6.

In one embodiment, each of the transgenes are operably linked to a tissue-specific promoter. References herein to the term “operably linked” include references to a DNA sequence and a regulatory sequence(s) are connected in such a way as to permit gene expression when the appropriate molecules (e.g., transcriptional activator proteins) are bound to the regulatory sequence(s).

The invention further provides a DNA construct comprising the QC transgene as described above. The invention also provides a DNA construct comprising the APP transgene as described above. As used herein, the term “DNA construct” refers to a specific arrangement of genetic elements in a DNA molecule.

References herein to the term “construct” includes a recombinant nucleic acid, generally recombinant DNA, that has been generated for the purpose of the expression of a specific nucleotide sequence(s), or is to be used in the construction of other recombinant nucleotide sequences. The recombinant nucleic acid can encode e.g. a chimeric or humanized polypeptide.

If desired, the DNA constructs can be engineered to be operatively linked to appropriate expression elements such as promoters or enhancers to allow expression of a genetic element in the DNA construct in an appropriate cell or tissue. The use of the expression control mechanisms allows for the targeted delivery and expression of the gene of interest. For example, the constructs of the present invention may be constructed using an expression cassette which includes in the 5′-3′ direction of transcription, a transcriptional and translational initiation region associated with gene expression in brain tissue, DNA encoding a mutant or wild-type QC or APP protein, and a transcriptional and translational termination region functional in the host animal. One or more introns also can be present. The transcriptional initiation region can be endogenous to the host animal or foreign or exogenous to the host animal.

The DNA constructs described herein, may be incorporated into vectors for propagation or transfection into appropriate cells to generate APP or QC overexpressing mutant non-human mammals and are also comprised by the present invention. One skilled in the art can select a vector based on desired properties, for example, for production of a vector in a particular cell such as a mammalian cell or a bacterial cell.

Vectors can contain a regulatory element that provides tissue specific or inducible expression of an operatively linked nucleic acid. One skilled in the art can readily determine an appropriate tissue-specific promoter or enhancer that allows expression of QC and APP polypeptides in a desired tissue. It should be noted that tissue-specific expression as described herein does not require a complete absence of expression in tissues other than the preferred tissue. Instead, “cell-specific” or “tissue-specific” expression refers to a majority of the expression of a particular gene of interest in the preferred cell type or tissue.

Any of a variety of inducible promoters or enhancers can also be included in the vector for expression of a QC or APP polypeptide or nucleic acid that can be regulated. Such inducible systems, include, for example, tetracycline inducible System (Gossen & Bizard, Proc. Natl. Acad. Sci. USA, 89:5547-5551 (1992); Gossen et al., Science, 268:17664769 (1995); Clontech, Palo Alto, Calif.); metallothionein promoter induced by heavy metals; insect steroid hormone responsive to ecdysone or related steroids such as muristerone (No et al., Proc. Natl. Acad. Sci. USA, 93:3346-3351 (1996); Yao et al., Nature, 366:476-479 (1993); Invitrogen, Carlsbad, Calif.); mouse mammary tumor virus (MMTV) induced by steroids such as glucocorticoid and estrogen (Lee et al., Nature, 294:228-232 (1981); and heat shock promoters inducible by temperature changes; the rat neuron specific enolase gene promoter (Forss-Petter, et al., Neuron 5; 197-197 (1990)); the human β-actin gene promoter (Ray, et al., Genes and Development (1991) 5:2265-2273); the human platelet derived growth factor B (PDGF-B) chain gene promoter (Sasahara, et al., Cell (1991) 64:217-227); the rat sodium channel gene promoter (Maue, et al., Neuron (1990) 4:223-231); the human copper-zinc superoxide dismutase gene promoter (Ceballos-Picot, et al., Brain Res. (1991) 552:198-214); and promoters for members of the mammalian POU-domain regulatory gene family (Xi et al., (1989) Nature 340:35-42).

Regulatory elements, including promoters or enhancers, can be constitutive or regulated, depending upon the nature of the regulation, and can be regulated in a variety of tissues, or one or a few specific tissues. The regulatory sequences or regulatory elements are operatively linked to one of the polynucleotide sequences of the invention such that the physical and functional relationship between the polynucleotide sequence and the regulatory sequence allows transcription of the polynucleotide sequence. Vectors useful for expression in eukaryotic cells can include, for example, regulatory elements including the CAG promoter, the SV40 early promoter, the cytomegalovirus (CMV) promoter, the mouse mammary tumor virus (MMTV) steroid-inducible promoter, Pgtf, Moloney marine leukemia virus (MMLV) promoter, thy-1 promoter and the like.

If desired, the vector can contain a selectable marker. As used herein, a “selectable marker” refers to a genetic element that provides a selectable phenotype to a cell in which the selectable marker has been introduced. A selectable marker is generally a gene whose gene product provides resistance to an agent that inhibits cell growth or kills a cell. A variety of selectable markers can be used in the DNA constructs of the invention, including, for example, Neo, Hyg, hisD, Gpt and Ble genes, as described, for example in Ausubel et al. (Current Protocols in Molecular Biology (Supplement 47), John Wiley & Sons, New York (1999)) and U.S. Pat. No. 5,981,830. Drugs useful for selecting for the presence of a selectable marker include, for example, G418 for Neo, hygromycin for Hyg, histidinol for hisD, xanthine for Gpt, and bleomycin for Ble (see Ausubel et al, supra, (1999); U.S. Pat. No. 5,981,830). DNA constructs of the invention can incorporate a positive selectable marker, a negative selectable marker, or both (see, for example, U.S. Pat. No. 5,981,830).

### Non-Human Transgenic Animals

The invention primarily provides a non-human transgenic animal whose genome comprises a transgene encoding a QC and APP polypeptide. References herein to the term “transgenic animal” include a non-human animal, a non-limiting example being a mammal, in that one or more of the cells of the animal includes a genetic modification as defined herein. Further non-limiting examples includes rodents such as a rat or mouse. Other examples of transgenic animals include non-human primates, sheep, dogs, cows, goats, chickens, amphibians etc. In one embodiment, the transgenic animal is a rodent such as a rat or mouse. In a further embodiment, the transgenic animal according to the present invention is a mouse.

It will be appreciated that the non-human transgenic animal of the invention may be obtained by crossbreeding a transgenic non-human animal overexpressing QC with a transgenic non-human animal for overexpressing APP. Thus, according to a further aspect of the invention there is provided a method of producing a transgenic non-human animal for overexpressing APP and QC, wherein said method comprises crossbreeding a transgenic non-human animal comprising cells containing a DNA transgene encoding human APP with a transgenic non-human animal comprising cells containing a DNA transgene encoding human QC. According to a further aspect of the invention there is provided a transgenic non-human animal for overexpressing APP and QC, obtainable by the method as hereinbefore defined.

In one embodiment, the animal is heterozygous for at least one of the transgenes, such as both transgenes. In an alternative embodiment, the animal is homozygous for at least one of the transgenes, such as both transgenes. In one embodiment, the animal is homozygous for APP and heterozygous for QC. In a further embodiment, the animal is a mouse.

The DNA fragment can be integrated into the genome of a transgenic animal by any method known to those skilled in the art. The DNA molecule containing the desired gene sequence can be introduced into pluripotent cells, such as ES cells, by any method that will permit the introduced molecule to undergo recombination at its regions of homology. Techniques that can be used include, but are not limited to, calcium phosphate/DNA co-precipitates, microinjection of DNA into the nucleus, electroporation, bacterial protoplast fusion with intact cells, transfection, and polycations, (e.g., polybrene, polyornithine, etc.) The DNA can be single or double stranded DNA, linear or circular. (See for example, Hogan et al., Manipulating the Mouse Embryo: A Laboratory Manual Cold Spring Harbor Laboratory (1986); Hogan et al., Manipulating the Mouse Embryo: A Laboratory Manual, second ed., Cold Spring Harbor Laboratory (1994), U.S. Pat. Nos. 5,602,299; 5,175,384; 6,066,778; 4,873,191 and 6,037,521; retrovirus mediated gene transfer into germ lines (Van der Putten et al., Proc. Natl. Acad. Sci. USA 82:6148-6152 (1985)); gene targeting in embryonic stem cells (Thompson et al., Cell 56:313-321 (1989)); electroporation of embryos (Lo, Mol Cell. Biol. 3:1803-1814 (1983)); and sperm-mediated gene transfer (Lavitrano et al., Cell 57:717-723 (1989)).

For example, the zygote is a good target for microinjection, and methods of microinjecting zygotes are well known (see U.S. Pat. No. 4,873,191).

Embryonal cells at various developmental stages can also be used to introduce transgenes for the production of transgenic animals. Different methods are used depending on the stage of development of the embryonal cell. Such transfected embryonic stem (ES) cells can thereafter colonize an embryo following their introduction into the blastocoele of a blastocyst-stage embryo and contribute to the germ line of the resulting chimeric animal (reviewed in Jaenisch, Science 240:1468-1474 (1988)). Prior to the introduction of transfected ES cells into the blastocoele, the transfected ES cells can be subjected to various selection protocols to enrich the proportion of ES cells that have integrated the transgene if the transgene provides a means for such selection. Alternatively, PCR can be used to screen for ES cells that have integrated the transgene.

In addition, retroviral infection can also be used to introduce transgenes into a non-human animal. The developing non-human embryo can be cultured in vitro to the blastocyst stage. During this time, the blastomeres can be targets for retroviral infection (Janenich, Proc. Nati. Acad. Sci. USA 73:1260-1264 (1976)). Efficient infection of the blastomeres is obtained by enzymatic treatment to remove the zona pellucida (Hogan et al., supra, 1986). The viral vector system used to introduce the transgene is typically a replication-defective retrovirus carrying the transgene (Jahner et al., Proc. Natl. Acad Sci. USA 82:6927-6931 (1985); Van der Putten et al., Proc. Natl. Acad Sci. USA 82:6148-6152 (1985)). Transfection is easily and efficiently obtained by culturing the blastomeres on a monolayer of virus-producing cells (Van der Putten, supra, 1985; Stewart et al., EMBO J. 6:383-388 (1987)). Alternatively, infection can be performed at a later stage. Virus or virus-producing cells can be injected into the blastocoele (Jahner D. et al., Nature 298:623-628 (1982)). Most of the founders will be mosaic for the transgene since incorporation occurs only in a subset of cells, which form the transgenic animal. Further, the founder can contain various retroviral insertions of the transgene at different positions in the genome, which generally will segregate in the offspring. In addition, transgenes may be introduced into the germline by intrauterine retroviral infection of the mid-gestation embryo (Jahner et al., supra, 1982). Additional means of using retroviruses or retroviral vectors to create transgenic animals known to those of skill in the art involves the micro-injection of retroviral particles or mitomycin C-treated cells producing retrovirus into the perivitelline space of fertilized eggs or early embryos (WO 90/08832 (1990); Haskell and Bowen, Mal. Reprod. Dev. 40:386 (1995)).

Any other technology to introduce transgenes into a non-human animal, e.g. the knock-in or the rescue technologies can also be used to solve the problem of the present invention. The knock-in technology is well known in the art as described e.g. in Casas et al. (2004) Am J Pathol 165, 1289-1300.

Once the founder animals are produced, they can be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic mice to produce mice homozygous for a given integration site in order to both augment expression and eliminate the need for screening of animals by DNA analysis; crossing of separate homozygous lines to produce compound heterozygous or homozygous lines; breeding animals to different inbred genetic backgrounds so as to examine effects of modifying alleles on expression of the transgene and the effects of expression.

The transgenic animals are screened and evaluated to select those animals having the phenotype of interest. Initial screening can be performed using, for example, Southern blot analysis or PCR techniques to analyze animal tissues to verify that integration of the transgene has taken place. The level of mRNA expression of the transgene in the tissues of the transgenic animals can also be assessed using techniques which include, but are not limited to, Northern blot analysis of tissue samples obtained from the animal, in situ hybridization analysis, and reverse transcriptase-PCR (rt-PCR). Samples of the suitable tissues can be evaluated immunocytochemically using antibodies specific for QC or with a tag such as EGFP. The transgenic non-human mammals can be further characterized to identify those animals having a phenotype useful in methods of the invention. In particular, transgenic non-human mammals overexpressing QC can be screened using the methods disclosed herein. For example, tissue sections can be viewed under a fluorescent microscope for die present of fluorescence, indicating the presence of the reporter gene.

Another method to affect tissue specific expression of the QC and APP protein is through the use of tissue-specific promoters. Non-limiting examples of suitable tissue-specific promoters include the albumin promoter (liver-specific; Pinkert et al., (1987) Genes Dev. 1:268-277); lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al., (1983) Cell 33:729-740; Queen and Baltimore (1983) Cell 33:741-748), neuron-specific promoters (e.g., the neurofilament promoter, the Thy-1 promoter or the Bri-protein promoter; Sturchler-Pierrat et al., (1997) Proc. Natl. Acad Sci. USA 94:13287-13292, Byrne and Ruddle (1989) PNAS 86:5473-5477), pancreas-specific promoters (Edlund et al., (1985) Science 230:912-916), cardiac specific expression (alpha myosin heavy chain promoter, Subramaniam, A, Jones W K, Gulick J, Wert S, Neumann J, and Robbins J. Tissue-specific regulation of the alpha-myosin heavy chain gene promoter in transgenic mice. J Biol Chem 266: 24613-24620, 1991.), and mammary gland-specific promoters (e.g., milk whey promoter; U.S. Pat. No. 4,873,316 and European Application Publication No. 264,166).

The invention further provides an isolated cell containing a DNA construct of the invention. The DNA construct can be introduced into a cell by any of the well-known transfection methods (Sambrook et al., Molecular Cloning: A Laboratory Manual, 2nd ed., Cold Spring Harbor Press, Plainview, N.Y. (1989); Ausubel et al., supra, (1999)). Alternatively, the cell can be obtained by isolating a cell from a mutant non-human mammal created as described herein. Thus, the invention provides a cell isolated from an APP and QC mutant non-human mammal of the invention, in particular, an APP and QC mutant mouse. The cells can be obtained from a homozygous APP and QC mutant non-human mammal such as a mouse or a heterozygous APP and QC mutant non-human mammal such as a mouse or a homozygous APP and heterozygous QC mutant non-human mammal such as a mouse.

According to a further aspect of the invention, there is provided a transgenic mouse comprising a transgenic nucleotide sequence encoding QC, which comprises the nucleotide sequence of SEQ ID NO: 4 or substantially the same nucleotide sequence of SEQ ID NO: 4, and a transgenic nucleotide sequence encoding APP, which comprises the nucleotide sequence of SEQ ID NOS: 5 or 6 or substantially the same nucleotide sequences of SEQ ID NOS: 5 or 6, operably linked to a promoter, integrated into the genome of the mouse, wherein the mouse demonstrates a phenotype that can be reversed or ameliorated with an QC inhibitor.

### Effectors

Effectors, as that term is used herein, are defined as molecules that bind to enzymes and increase (promote) or decrease (inhibit) their activity in vitro and/or in vivo. Some enzymes have binding sites for molecules that affect their catalytic activity; a stimulator molecule is called an activator. Enzymes may even have multiple sites for recognizing more than one activator or inhibitor. Enzymes can detect concentrations of a variety of molecules and use that information to vary their own activities.

Effectors can modulate enzymatic activity because enzymes can assume both active and inactive conformations: activators are positive effectors, inhibitors are negative effectors. Effectors act not only at the active sites of enzymes, but also at regulatory sites, or allosteric sites, terms used to emphasize that the regulatory site is an element of the enzyme distinct from the catalytic site and to differentiate this form of regulation from competition between substrates and inhibitors at the catalytic site (Darnell, J., Lodish, H. and Baltimore, D. 1990, Molecular Cell Biology 2nd Edition, Scientific American Books, New York, page 63).

### Assays and Identification of Therapeutic Agents

The methods and compositions of the present invention are particularly useful in the evaluation of effectors of QC, preferably activity decreasing effectors of QC, i.e. QC inhibitors, and for the development of drugs and therapeutic agents for the treatment and prevention of a disease selected from mild cognitive impairment, Alzheimer's disease, Familial British Dementia, Familial Danish Dementia, neurodegeneration in Down Syndrome, Huntington's disease, Kennedy's disease, ulcer disease, duodenal cancer with or w/o Helicobacter pylori infections, colorectal cancer, Zolliger-Ellison syndrome, gastric cancer with or without Helicobacter pylori infections, pathogenic psychotic conditions, schizophrenia, infertility, neoplasia, inflammatory host responses, cancer, malign metastasis, melanoma, psoriasis, rheumatoid arthritis, atherosclerosis, pancreatitis, restenosis, lung fibrosis, liver fibrosis, renal fibrosis, graft rejection, acquired immune deficiency syndrome, impaired humoral and cell-mediated immune responses, leukocyte adhesion and migration processes in the endothelium, impaired food intake, impaired sleep-wakefulness, impaired homeostatic regulation of energy metabolism, impaired autonomic function, impaired hormonal balance or impaired regulation of body fluids, multiple sclerosis, the Guillain-Barré syndrome and chronic inflammatory demyelinizing polyradiculoneuropathy.

The transgenic animal or the cells of the transgenic animal of the invention can be used in a variety of screening assays. Thus, according to a further aspect of the invention, there is provided a method of screening for biologically active agents that inhibit or promote QC production in vivo, comprising:
(a) administering a test agent to the transgenic non-human animal as defined herein; and
(b) determining the effect of the agent on the amount of QC produced or on the QC activity.

According to a yet further aspect of the invention there is provided a method of screening for therapeutic agents that inhibit or promote QC activity comprising:
(a) administering test agents to the transgenic non-human animal as defined herein;
(b) evaluating the effects of the test agent on the neurological phenotype of the transgenic non-human animal; and
(c) selecting a test agent which inhibits or promotes QC activity.

For example, any of a variety of potential agents suspected of affecting QC and amyloid accumulation, as well as the appropriate antagonists and blocking therapeutic agents, can be screened by administration to the transgenic animal and assessing the effect of these agents upon the function and phenotype of the cells and on the (neurological) phenotype of the transgenic animals.

Behavioral studies may also be used to test potential therapeutic agents, such as those studies designed to assess motor skills, learning and memory deficits. An example of such a test is the Morris Water maze (Morris (1981) Learn Motivat 12:239-260). Additionally, behavioral studies may include evaluations of locomotor activity such as with the rotor-rod and the open field.

The methods of the invention can advantageously use cells isolated from a homozygous or heterozygous APP and QC mutant non-human mammal, to study amyloid accumulation as well as to test potential therapeutic compounds. The methods of the invention can also be used with cells expressing APP and QC such as a transfected cell line.

According to a further aspect of the invention, there is provided a cell or cell line derived from the transgenic non-human animal as defined herein.

A cell overexpressing APP and QC can be used in an in vitro method to screen compounds as potential therapeutic agents for treating a QC-related disease, preferably a neurodegenerative disease, more preferably a disease selected from Mild Cognitive Impairment, Alzheimer's disease, cerebral amyloid angiopathy, Lewy body dementia, neurodegeneration in Down Syndrome, hereditary cerebral hemorrhage with amyloidosis (Dutch type), Familial Danish Dementia, Familial British Dementia and Chorea Huntington. In such a method, a compound is contacted with a cell overexpressing APP and QC, a transfected cell or a cell derived from a APP and QC mutant non-human animal which overexpresses APP and QC, and screened for alterations in a phenotype associated with expression of APP and QC. The changes in Aβ production, preferably the production of N-terminal truncated forms of Aβ, more preferably the production of N-terminal truncated forms of Aβ starting at amino acid position no. 3, such as Aβ(3-40), Aβ(3-42) and Aβ(3-43), or starting at amino acid position no. 11, such as Aβ(11-40), Aβ(11-42) and Aβ(11-43), most preferably N-terminal truncated forms of Aβ starting with a pyroglutamate (pGlu) residue at amino acid position no. 3 or no. 11, such as pGlu-Aβ(3-40), pGlu-Aβ(3-42), pGlu-Aβ(3-43), pGlu-Aβ(11-40), pGlu-Aβ(11-42) and pGlu-Aβ(11-43) in the cellular assay and the transgenic animal can be assessed by methods well known to those skilled in the art.

A QC and/or APP fusion polypeptide such as green fluorescent protein can be particularly useful for such screening methods since the expression of QCand/or APP can be monitored by fluorescence intensity. Other exemplary fusion polypeptides include other fluorescent proteins, or modifications thereof, glutathione S transferase (GST), maltose binding protein, poly His, and the like, or any type of epitope tag. Such fusion polypeptides can be detected, for example, using antibodies specific to the fusion polypeptides. The fusion polypeptides can be an entire polypeptide or a functional portion thereof so long as the functional portion retains desired properties, for example, antibody binding activity or fluorescence activity.

The invention further provides a method of identifying a potential therapeutic agent for use in treating the diseases as mentioned above. The method includes the steps of contacting a cell containing one or more DNA constructs comprising polynucleotides encoding an APP and a QC polypeptide with a compound and screening the cell for one or more of decreased QC production, decreased enzymatic activity of QC, decreased APP production, decreased Aβ production, preferably decreased production of N-terminal truncated forms of Aβ, more preferably decreased production of N-terminal truncated forms of Aβ starting at amino acid position no. 3, such as Aβ(3-40), Aβ(3-42) and Aβ(3-43), or starting at amino acid position no. 11, such as Aβ(11-40), Aβ(11-42) and Aβ(11-43), most preferably decreased production of N-terminal truncated forms of Aβ starting with a pyroglutamate (pGlu) residue at amino acid position no. 3 or no. 11, such as pGlu-Aβ(3-40), pGlu-Aβ(3-42), pGlu-Aβ(3-43), pGlu-Aβ(11-40), pGlu-Aβ(11-42) and pGlu-Aβ(11-43), thereby identifying a potential therapeutic agent for use in treating QC-related diseases. The cell can be isolated from a transgenic non-human mammal having nucleated cells containing the QC and APP DNA construct. Alternatively, the cell can contain a DNA construct comprising a nucleic acid encoding a green fluorescent protein fusion, or other fusion polypeptide, with a QC polypeptide.

Additionally, cells expressing an APP and a QC polypeptide can be used in a preliminary screen to identify compounds as potential therapeutic agents having activity that alters a phenotype associated with QC expression. As with in vivo screens using the APP and QC transgenic non-human mammals, an appropriate control cell can be used to compare the results of the screen. The effectiveness of compounds identified by an initial in vitro screen using cells expressing APP and QC can be further tested in vivo using the APP and QC transgenic non-human mammals of the invention, if desired. Thus, the invention provides methods of screening a large number of compounds using a cell-based assay, for example, using high throughput screening, as well as methods of further testing compounds as therapeutic agents in an animal model of Aβ-related disorders.

The non-human transgenic animals whose genome comprises a transgene encoding a QC polypeptide can be used to investigate the physiological function of QC in vivo.

In a preferred embodiment, the APP and QC transgenic animals of the present invention are crossbred with existing animal models that are acknowledged disease specific animal models. Such crossbred animals can be used to determine the effect of overexpressed recombinant APP and QC and/or increased APP and QC activity on the outbreak, course and severity of said specific diseases.

A suitable method comprises the following steps:
(a) Crossbreeding of the APP and QC transgenic non-human animals of the present invention with a non-human animal model, which is specific for a desired disease,
(b) Breeding and ageing the crossbred animals and the disease specific animals;
(c) Monitoring the disease state age-dependently in the crossbred animals,
(d) As a control group, monitoring the disease state age-dependently in the disease specific animal models that are not transgenic for APP and QC,
(e) Calculating the differences in the disease state in the crossbred animals versus the disease specific anmals, and
(f) Determining the effect of the APP and QC transgenes on the disease state.

To determine the effect of the APP and QC transgenes on the disease state, the increase of the production of APP and/or Aβ, preferably increased production of N-terminal truncated forms of Aβ, more preferably increased production of N-terminal truncated forms of Aβ starting at amino acid position no. 3, such as Aβ(3-40), Aβ(3-42) and Aβ(3-43), or starting at amino acid position no. 11, such as Aβ(11-40), Aβ(11-42) and Aβ(11-43), most preferably increased production of N-terminal truncated forms of Aβ starting with a pyroglutamate (pGlu) residue at amino acid position no. 3 or no. 11, such as pGlu-Aβ(3-40), pGlu-Aβ(3-42), pGlu-Aβ(3-43), pGlu-Aβ(11-40), pGlu-Aβ(11-42) and pGlu-Aβ(11-43) can be measured in the aforementioned method with conventional assays.

Furthermore, said crossbred animals are suitable for use in methods of screening for activity decreasing effectors of QC (QC inhibitors). A suitable screening method comprises:
(a) Crossbreeding of the APP and QC transgenic non-human animals of the present invention with a non-human animal model, which is specific for a desired disease,
(b) Administering a test agent to a treatment group of crossbred animals,
(c) Administering a placebo to a control group of crossbred animals,
(d) Monitoring the disease state age-dependently in the crossbred animals,
(e) Monitoring the disease state age-dependently in the control group,
(f) Calculating the differences in the disease state in the treatment group versus the control group, and
(g) Determining the effect of the test agent on the disease state.

Suitably, the effect of the test agent investigated in the aforementioned method is one of decreased enzymatic activity of QC, decreased APP production, decreased Aβ production, preferably decreased production of N-terminal truncated forms of Aβ, more preferably decreased production of N-terminal truncated forms of Aβ starting at amino acid position no. 3, such as Aβ(3-40), Aβ(3-42) and Aβ(3-43), or starting at amino acid position no. 11, such as Aβ(11-40), Aβ(11-42) and Aβ(11-43), most preferably decreased production of N-terminal truncated forms of Aβ starting with a pyroglutamate (pGlu) residue at amino acid position no. 3 or no. 11, such as pGlu-Aβ(3-40), pGlu-Aβ(3-42), pGlu-Aβ(3-43), pGlu-Aβ(11-40), pGlu-Aβ(11-42) and pGlu-Aβ(11-43), each of which can be measured with conventional assays.

Suitably, the crossbred animals are heterozygous for the APP and QC transgenes. Also suitably, the crossbred animals are homozygous for the APP and QC transgenes. In one embodiment, the crossbred animals are homozygous for the APP transgene and heterozygous for the QC transgene.

The recombinant APP and QC, which are overexpressed in the aforementioned crossbred non-human animals, suitably leads to one or more of the following effects on the disease state: an earlier outbreak of the specific disease, an accelerated course of the specific disease and/or a more severe course of the specific disease.

Another effect of the overexpressed APP and QC could be the increase or decrease of the level of one or more QC substrates in the crossbred hon-human animals. Preferred QC substrates are of N-terminal truncated forms of Aβ, more preferably N-terminal truncated forms of Aβ starting at amino acid position no. 3, such as Aβ(3-40), Aβ(3-42) and Aβ(3-43), or starting at amino acid position no. 11, such as Aβ(11-40), Aβ(11-42) and Aβ(11-43.

A particular preferred embodiment is the use of this method for screening of QC inhibitors.

Suitably, this method is used for the screening of QC inhibitors for the treatment of a disease selected from mild cognitive impairment, Alzheimer's disease, Familial British Dementia, Familial Danish Dementia, neurodegeneration in Down Syndrome, Huntington's disease, Kennedy's disease, ulcer disease, duodenal cancer with or w/o Helicobacter pylori infections, colorectal cancer, Zolliger-Ellison syndrome, gastric cancer with or without Helicobacter pylori infections, pathogenic psychotic conditions, schizophrenia, infertility, neoplasia, inflammatory host responses, cancer, malign metastasis, melanoma, psoriasis, rheumatoid arthritis, atherosclerosis, pancreatitis, restenosis, lung fibrosis, liver fibrosis, renal fibrosis, graft rejection, acquired immune deficiency syndrome, impaired humoral and cell-mediated immune responses, leukocyte adhesion and migration processes in the endothelium, impaired food intake, impaired sleep-wakefulness, impaired homeostatic regulation of energy metabolism, impaired autonomic function, impaired hormonal balance or impaired regulation of body fluids, multiple sclerosis, the Guillain-Barré syndrome and chronic inflammatory demyelinizing polyradiculoneuropathy.

In a further preferred embodiment, this method is used for the screening of QC inhibitors for the treatment of Alzheimer's disease or neurodegeneration in Down syndrome.

In yet another preferred embodiment, this method is used for the screening of QC inhibitors for the treatment of Familial British Dementia or Familial Danish Dementia.

Furthermore, this method is preferably used for the screening of QC inhibitors for the treatment of a disease selected from rheumatoid arthritis, atherosclerosis, restenosis, and pancreatitis.

The efficacy of QC inhibitors for the treatment of Alzheimer's Disease, Familial British Dementia or Familial Danish Dementia and, e.g. neurodegeneration in Down Syndrome can be tested in existing animal models of Alzheimer's disease.

QC may be involved in the formation of pyroglutamic acid that favors the aggregation of amyloid β-peptides. Therefore, a suitable QC substrate, which can be monitored when the above methods are employed, is one selected from [Glu3]Aβ3-40/42/43 or [Glu11]Aβ11-40/42/43. These peptides are involved in the onset and progression of Alzheimer's disease and neurodegeneration in Down Syndrome. Recombinant QC, which is expressed in the crossbred non-human animals of the present invention, may lead to one or more of the following effects: earlier formation of at least one of [pGlu3]Aβ3-40/42/43 or [pGlu11]Aβ11-40/42/43, faster formation of at least one of [pGlu3]Aβ3-40/42/43 or [pGlu11]Aβ11-40/42/43 or increased level of at least one of [pGlu3]Aβ3-40/42/43 or [pGlu11]Aβ11-40/42/43.

The QC inhibitor, which is selected by employing the screening method in the crossbred non-human animals accordingly leads to the prevention of the formation of at least one of [pGlu3]Aβ3-40/42/43 or [pGlu11]Aβ3-40/42/43 and may subsequently lead to the prevention of the precipitation of amyloid β-peptides and formation of plaques. Finally, said QC inhibitor should suitably lead to one or more of the following effects: postponing the outbreak, slowing down the course and/or reducing the severity of Alzheimer's disease and neurodegeneration in Down Syndrome in the crossbred non-human animals.

Suitable animal models of Alzheimer's Disease are reviewed in McGowan et al., TRENDS in Genetics, Vol. 22, No. May 2006, pp 281-289, and are selected from PDAPP, Tg2576, APP23, TgCRND8, PSEN1M146V or PSEN1M146L, PSAPP, APPDutch, BRI-Aβ40 and BRI-Aβ42, JNPL3, TauP301S, TauV337M, TauR406W, rTg4510, Htau, TAPP, 3×TgAD, as described below. Another suitable model of Alzheimer's disease is the 5XFAD model (Oakley H., et al., Intraneuronal beta-amyloid aggregates, neurodegeneration, and neuron loss in transgenic mice with five familial Alzheimer's disease mutations: potential factors in amyloid plaque formation. J Neurosci. 2006 Oct. 4; 26(40):10129-40).

PDAPP: First mutant APP transgenic model with robust plaque pathology. Mice express a human APP cDNA with the Indiana mutation (APPV717F). Plaque pathology begins between 6-9 months in hemizygous PDAPP mice. There is synapse loss but no overt cell loss and no NFT pathology is observed. This model has been used widely in vaccination therapy strategies.

Tg2576: Mice express mutant APPSWE under control of the hamster prion promoter. Plaque pathology is observed from 9 months of age. These mice have cognitive deficits but no cell loss or NFT pathology. It is one of the most widely used transgenic models.

APP23: Mice express mutant APPSWE under control of the Thy1 promoter. Prominent cerebrovascular amyloid, amyloid deposits are observed from 6 months of age and some hippocampal neuronal loss is associated with amyloid plaque formation.

TgCRND8: Mice express multiple APP mutations (Swedish plus Indiana). Cognitive deficits coincide with rapid extracellular plaque development at ˜3 months of age. The cognitive deficits can be reversed by Aβ vaccination therapy.

PSEN1M146V or PSEN1M146L (lines 6.2 and 8.9, respectively): These models were the first demonstration in vivo that mutant PSEN1 selectively elevates Aβ42. No overt plaque pathology is observed.

PSAPP (Tg2576×PSEN1M146L, PSEN1-A246E+APPSWE): Bigenic transgenic mice, addition of the mutant PSEN1 transgene markedly accelerated amyloid pathology compared with singly transgenic mutant APP mice, demonstrating that the PSEN1-driven elevation of Aβ42 enhances plaque pathology.

APPDutch: Mice express APP with the Dutch mutation that causes hereditary cerebral hemorrhage with amyloidosis-Dutch type in humans. APPDutch mice develop severe congophilic amyloid angiopathy. The addition of a mutant PSEN1 transgene redistributes the amyloid pathology to the parenchyma indicating differing roles for Aβ40 and Aβ42 in vascular and parenchymal amyloid pathology.

BRI-Aβ40 and BRI-Aβ42: Mice express individual Aβ isoforms without APP over-expression. Only mice expressing Aβ42 develop senile plaques and CAA, whereas BRI-Aβ40 mice do not develop plaques, suggesting that Aβ42 is essential for plaque formation.

JNPL3: Mice express 4RON MAPT with the P301 L mutation. This is the first transgenic model, with marked tangle pathology and cell loss, demonstrating that MAPT alone can cause cellular damage and loss. JNPL3 mice develop motor impairments with age owing to servere pathology and motor neuron loss in the spinal cord.

TauP301 S: Tansgenic mice expressing the shortest isoform of 4R MAPT with the P301 S mutation. Homozygous mice develop severe paraparesis at 5-6 months of age with widespread neurofibrillary pathology in the brain and spinal cord and neuronal loss in the spinal cord.

TauV337M: Low level synthesis of 4R MAPT with the V337M mutation ( 1/10 endogenous MAPT) driven by the promoter of platelet-derived growth factor (PDGF). The development of neurofibrillary pathology in these mice suggests the nature of the MAPT rather than absolute MAPT intracellular concentration drives pathology.

TauR406W: Mice expressing 4R human MAPT with the R406W mutation under control of the CAMKII promoter. Mice develop MAPT inclusions in the forebrain from 18 months of age and have impaired associative memory.

rTg4510: Inducible MAPT transgenic mice using the TET-off system. Abnormal MAPT pathology occurs from one month of age. Mice have progressive NFT pathology and severe cell loss. Cognitive deficits are evident from 2.5 months of age. Turning off the transgene improves cognitive performance but NT pathology worsens.

Htau: Transgenic mice expressing human genomic MAPT only (mouse MAPT knocked-out). Htau mice accumulate hyperphosphorylated MAPT from 6 months and develop Thio-S-positive NFT by the time they are 15 months old.

TAPP (Tg2576×JNPL3): Increased MAPT forebrain pathology in TAPP mice compared with JNPL3 suggesting mutant APP and/or Aβ can affect downstream MAPT pathology.

3×TgAD: Triple transgenic model expressing mutant APPSWE, MAPTP301 L on a PSEN1M146V ‘knock-in’ background (PSNE1-KI). Mice develop plaques from 6 months and MAPT pathology from the time they are 12 months old, strengthening the hypothesis that APP or Aβ can directly influence neurofibrillary pathology.

5XFAD: Mutations in the genes for amyloid precursor protein (APP) and presenilins (PS1, PS2) increase production of beta-amyloid 42 (Abeta42) and cause familial Alzheimer's disease (FAD). Transgenic mice that express FAD mutant APP and PS1 overproduce Abeta42 and exhibit amyloid plaque pathology similar to that found in AD, but most transgenic models develop plaques slowly. To accelerate plaque development and investigate the effects of very high cerebral Abeta42 levels, APP/PS1 double transgenic mice were generated that coexpress five FAD mutations (5XFAD mice) and additively increase Abeta42 production. 5XFAD mice generate Abeta42 almost exclusively and rapidly accumulate massive cerebral Abeta42 levels. Amyloid deposition (and gliosis) begins at 2 months and reaches a very large burden, especially in subiculum and deep cortical layers. Intraneuronal Abeta42 accumulates in 5XFAD brain starting at 1.5 months of age (before plaques form), is aggregated (as determined by thioflavin S staining), and occurs within neuron soma and neurites. Some amyloid deposits originate within morphologically abnormal neuron soma that contain intraneuronal Abeta. Synaptic markers synaptophysin, syntaxin, and postsynaptic density-95 decrease with age in 5XFAD brain, and large pyramidal neurons in cortical layer 5 and subiculum are lost. In addition, levels of the activation subunit of cyclin-dependent kinase 5, p25, are elevated significantly at 9 months in 5XFAD brain. Finally, 5XFAD mice have impaired memory in the Y-maze.

Suitable study designs are conventional. QC inhibitors could be applied via the drinking solution or chow, or any other conventional route of administration, e.g. orally, intravenously or subcutaneously.

In regard to Alzheimer's disease and neurodegeneration in Down syndrome, the efficacy of the QC inhibitors can be assayed by sequential extraction of Aβ using SDS and formic acid. Initially, the SDS and formic acid fractions containing the highest Aβ concentrations can be analyzed using an ELISA quantifying total Aβ(x-42) or Aβ(x-40) as well as pGluAβ3-40/42/43 or pGluAβ11-40/42/43. In particular, suitable QC inhibitors are capable to reduce the formation of pGluAβ3-40 and/or pGluAβ3-42. Even preferred are QC inhibitors that are capable to reduce the formation of pGluAβ11-40 and/or pGluAβ11-42.

An ELISA kit for the quantification of pGluAβ3-42 is commercially available from IBL, Cat-no. JP27716.

An ELISA for the quantification of pGluAβ3-40 is described by Schilling et al., 2008 (Schilling S, Appl T, Hoffmann T, Cynis H, Schulz K, Jagla W, Friedrich D, Wermann M, Buchholz M, Heiser U, von Hörsten S, Demuth H U. Inhibition of glutaminyl cyclase prevents pGlu-Abeta formation after intracortical/hippocampal microinjection in vivo/in situ. J Neurochem. 2008 August; 106(3):1225-36.)

Subsequently after QC inhibitor treatment, the crossbred non-human animals can be tested regarding behavioral changes. Suitable behavioral test paradigms are, e.g. those, which address different aspects of hippocampus-dependent learning. Examples of such neurological tests are the Morris water maze test and the Fear Conditioning test looking at contextual memory changes (Comery, T A et al, (2005), J Neurosci 25:8898-8902; Jacobsen J S et al, (2006), Proc Natl. Acad. Sci USA 103:5161-5166). Further suitable behavioral tests are outlined in the working examples of the present application. Suitably, the QC inhibitors, which are selected by employing the screening methods of the present invention, reduce the behavioral changes, or more suitably improve the behavior of the crossbred non-human animals.

The animal model of inflammatory diseases, e.g. atherosclerosis contemplated by the present invention can be an existing atherosclerosis animal model, e.g., the apoE deficient mouse. The apolipoprotein E knockout mouse model has become one of the primary models for atherosclerosis (Arterioscler Thromh Vase Biol., 24: 1006-1014, 2004; Trends Cardiovasc Med, 14: 187-190, 2004). The studies with the crossbred non-human animals of the present invention may be performed as described by Johnson et al. in Circulation, 111: 1422-1430, 2005, or using modifications thereof. Apolipoprotein E-Deficient Mouse Model Apolipoprotein E (apoE) is a component of several plasma lipoproteins, including chylomicrons, VLDL, and HDL. Receptor-mediated catabolism of these lipoprotein particles is mediated through the interaction of apoE with the LDL receptor (LDLR) or with LDLR-related protein (LRP). ApoE-deficient mice exhibit hypercholesterolemia and develop complex atheromatous lesions similar to those seen in humans. The efficacy of the compounds of the present invention was also evaluated using this animal model.

Other animal models for inflammatory diseases, which are suitable for use in the aforementioned screening method, include those where inflammation is initiated by use of an artificial stimulus. Such animal models are the thioglycollate-induced inflammation model, the collagen-induced arthritis model, the antibody induced arthritis model and models of restenosis (e.g. the effects of the test compounds on rat carotid artery responses to the balloon catheter injury). Such artificial stimuli can be used to initiate an inflammatory response in the crossbred non-human animal models of the present invention.

In inflammatory diseases, chemotactic cytokines play a role. Chemotactic cytokines (chemokines) are proteins that attract and activate leukocytes and are thought to play a fundamental role in inflammation. Chemokines are divided into four groups categorized by the appearance of N-terminal cysteine residues (“C”-; “CC”-; “CXC”- and “CX3C”-chemokines). “CXC”-chemokines preferentially act on neutrophils. In contrast, “CC”-chemokines attract preferentially monocytes to sites of inflammation. Monocyte infiltration is considered to be a key event in a number of disease conditions (Gerard, C. and Rollins, B. J. (2001) Nat. Immunol 2, 108-115; Bhatia, M., et al., (2005) Pancreatology. 5, 132-144; Kitamoto, S., Egashira, K., and Takeshita, A. (2003) J Pharmacol Sci. 91, 192-196). The MCP family, as one family of chemokines, consists of four members (MCP-1-4), displaying a preference for attracting monocytes but showing differences in their potential (Luini, W., et al., (1994) Cytokine 6, 28-31; Uguccioni, M., et al., (1995) Eur J Immunol 25, 64-68). The chemokines CCL2 (MCP-1), CCL8 (MCP-2), CCL7 (MCP-3), CCL13 (MCP-1), CCL16, CCL18 bear a glutamine (Gln) residue at the N-terminus and are therefore substrates of QC.

Accordingly, QC may be involved in the formation of pyroglutamic acid at the N-terminus of the chemokines CCL2, CCL8, CCL7, CCL13, CCL 16, and CCL 18 that stabilizes these chemokines against degradation by proteases and aminopeptidases and thereby maintains their biological activity in chemotaxis. Recombinant QC, which is expressed in the crossbred non-human animals of the present invention, may lead to one or more of the following effects: earlier formation of at least one of [pGlu1]CCL2, [pGlu1]CCL8, [pGlu1]CCL7, [pGlu1]CCL13, [pGlu1]CCL 16, or [pGlu1]CCL 18, faster formation of at least one of [pGlu1]CCL2, [pGlu1]CCL8, [pGlu1]CCL7, [pGlu1]CCL13, [pGlu1]CCL 16, or [pGlu1]CCL 18 or increased level of at least one of [pGlu1]CCL2, [pGlu1]CCL8, [pGlu1]CCL7, [pGlu1]CCL13, [pGlu1]CCL 16, or [pGlu1]CCL 18.

The QC inhibitor, which is selected by employing the screening method in the crossbred non-human animals accordingly leads to the prevention of the formation of at least one of [pGlu1]CCL2, [pGlu1]CCL8, [pGlu1]CCL7, [pGlu1]CCL13, [pGlu1]CCL 16, or [pGlu1]CCL 18.

The efficacy of the QC inhibitors can be assayed by measuring the inhibition of the chemotaxis of a monocytic cells induced by MCP-1 in vitro and in vivo or by measuring the inflammatory response caused by thioglycollate, collagen, antibody or LPS induction. Effective QC inhibitors should show a reduced monocyte infiltration after thioglycollate, collagen, antibody or LPS induction of inflammation.

Furthermore, the inhibition of the formation of [pGlu1]CCL2, [pGlu1]CCL8, [pGlu1]CCL7, [pGlu1]CCL13, [pGlu1]CCL 16, or [pGlu1]CCL 18 can be tested in vitro and in vivo.

In one embodiment, the present invention provides the use of activity-decreasing effectors of QC, as selected with use of the present inventive animal model, for the suppression of pGlu-Amyloid peptide formation in Mild Cognitive Impairment, Alzheimer's disease, Down Sydrome, Famlilial Danish Dementia and Familial British Dementia.

In a further embodiment, the present invention provides the use of activity-increasing effectors of QC, as selected with use of the present inventive animal model, for the stimulation of gastrointestinal tract cell proliferation, especially gastric mucosal cell proliferation, epithelial cell proliferation, the differentiation of acid-producing parietal cells and histamine-secreting enterochromaffin-like (ECL) cells, and the expression of genes associated with histamine synthesis and storage in ECL cells, as well as for the stimulation of acute acid secretion in mammals by maintaining or increasing the concentration of active[pGlu¹]-Gastrin.

In a further embodiment, the present invention provides the use of activity decreasing effectors of QC, as selected with use of the present inventive animal model, for the treatment of duodenal ulcer disease and gastric cancer with or without Helicobacter pylori in mammals by decreasing the conversion rate of inactive [Gln¹]Gastrin to active [pGlu¹]Gastrin.

In another embodiment, the present invention provides the use of activity increasing effectors of QC, as selected with use of the present inventive animal model, for the preparation of antipsychotic drugs and/or for the treatment of schizophrenia in mammals. The effectors of QC either maintain or increase the concentration of active [pGlu^{l}]neurotensin.

In a further embodiment, the present invention provides the use of activity-lowering effectors of QC, as selected with the present inventive animal model, for the preparation of fertilization prohibitive drugs and/or to reduce the fertility in mammals. The activity lowering effectors of

QC decrease the concentration of active [pGlu¹]FPP, leading to a prevention of sperm capacitation and deactivation of sperm cells. In contrast it could be shown that activity-increasing effectors of QC are able to stimulate fertility in males and to treat infertility.

In another embodiment, the present invention provides the use of effectors of QC, as selected with use of the present inventive animal model, for the preparation of a medicament for the treatment of pathophysiological conditions, such as suppression of proliferation of myeloid progenitor cells, neoplasia, inflammatory host responses, cancer, malign metastasis, melanoma, psoriasis, rheumatoid arthritis, atherosclerosis, lung fibrosis, liver fibrosis, renal fibrosis, graft rejection, acquired immune deficiency syndrom, impaired humoral and cell-mediated immunity responses, leukocyte adhesion and migration processes at the endothelium.

In a further embodiment, the present invention provides the use of effectors of QC, as selected with use of the present inventive animal model, for the preparation of a medicament for the treatment of impaired food intake and sleep-wakefulness, impaired homeostatic regulation of energy metabolism, impaired autonomic function, impaired hormonal balance and impaired regulation of body fluids.

In a further embodiment, the present invention therefore provides the use of effectors of QC, as selected with the present inventive animal model, for the preparation of a medicament for the treatment of Parkinson disease and Huntington's disease.

In another embodiment, the present invention provides a general way to reduce or inhibit the enzymatic activity of QC by using the test agent selected above.

The agents selected by the above-described screening methods can work by decreasing the conversion of at least one substrate of QC (negative effectors, inhibitors), or by increasing the conversion of at least one substrate of QC (positive effectors, activators).

According to a further aspect of the invention, there is provided a method of the treatment or prevention of a QC-related disease comprising:
(a) administering the selected test agent as defined herein; and
(b) monitoring the patient for a decreased clinical index for QC-related diseases.

In one embodiment, the QC-related disease is selected from Mild Cognitive Impairment, Alzheimer's disease, cerebral amyloid angiopathy, Lewy body dementia, neurodegeneration in Down Syndrome, hereditary cerebral hemorrhage with amyloidosis (Dutch type), Familial Danish Dementia, Familial British Dementia and Chorea Huntington.

In another embodiment, the QC-related disease is MCI or AD.

According to a further aspect of the invention, there is provided a test agent as defined herein for use in the treatment and/or prevention of a QC-related disease, such as Mild Cognitive Impairment, Alzheimer's disease, cerebral amyloid angiopathy, Lewy body dementia, neurodegeneration in Down Syndrome, hereditary cerebral hemorrhage with amyloidosis (Dutch type), Familial Danish Dementia, Familial British Dementia or Chorea Huntington.

The compounds of the present invention can be converted into acid addition salts, especially pharmaceutically acceptable acid addition salts.

The salts of the compounds of the invention may be in the form of inorganic or organic salts.

The compounds of the present invention can be converted into and used as acid addition salts, especially pharmaceutically acceptable acid addition salts. The pharmaceutically acceptable salt generally takes a form in which a basic side chain is protonated with an inorganic or organic acid. Representative organic or inorganic acids include hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. All pharmaceutically acceptable acid addition salt forms of the compounds of the present invention are intended to be embraced by the scope of this invention.

In view of the close relationship between the free compounds and the compounds in the form of their salts, whenever a compound is referred to in this context, a corresponding salt is also intended, provided such is possible or appropriate under the circumstances.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms of the compounds may exist as polymorphs and as such are intended to be included in the present invention. In addition, some of the compounds may form solvates with water (i.e. hydrates) or common organic solvents, and such solvates are also intended to be encompassed within the scope of this invention.

The compounds, including their salts, can also be obtained in the form of their hydrates, or include other solvents used for their crystallization.

In a further embodiment, the present invention provides a method of preventing or treating a condition mediated by modulation of the QC enzyme activity in a subject in need thereof which comprises administering any of the compounds of the present invention or pharmaceutical compositions thereof in a quantity and dosing regimen therapeutically effective to treat the condition. Additionally, the present invention includes the use of the compounds of this invention, and their corresponding pharmaceutically acceptable acid addition salt forms, for the preparation of a medicament for the prevention or treatment of a condition mediated by modulation of the QC activity in a subject. The compound may be administered to a patient by any conventional route of administration, including, but not limited to, intravenous, oral, subcutaneous, intramuscular, intradermal, parenteral and combinations thereof.

In a further preferred form of implementation, the invention relates to pharmaceutical compositions, that is to say, medicaments, that contain at least one compound or test agent as defined herein or salts thereof, optionally in combination with one or more pharmaceutically acceptable carriers and/or solvents. The pharmaceutical compositions may, for example, be in the form of parenteral or enteral formulations and contain appropriate carriers, or they may be in the form of oral formulations that may contain appropriate carriers suitable for oral administration. Preferably, they are in the form of oral formulations.

The effectors of QC activity administered according to the invention may be employed in pharmaceutically administrable formulations or formulation complexes as inhibitors or in combination with inhibitors, substrates, pseudosubstrates, inhibitors of QC expression, binding proteins or antibodies of those enzyme proteins that reduce the QC protein concentration in mammals. The compounds of the invention make it possible to adjust treatment individually to patients and diseases, it being possible, in particular, to avoid individual intolerances, allergies and side-effects.

The compounds also exhibit differing degrees of activity as a function of time. The physician providing treatment is thereby given the opportunity to respond differently to the individual situation of patients: he is able to adjust precisely, on the one hand, the speed of the onset of action and, on the other hand, the duration of action and especially the intensity of action.

A preferred treatment method according to the invention represents a new approach for the prevention or treatment of a condition mediated by modulation of the QC enzyme activity in mammals. It is advantageously simple, susceptible of commercial application and suitable for use, especially in the treatment of diseases that are based on unbalanced concentration of physiological active QC substrates in mammals and especially in human medicine.

The compounds may be advantageously administered, for example, in the form of pharmaceutical preparations that contain the active ingredient in combination with customary additives like diluents, excipients and/or carriers known from the prior art. For example, they can be administered parenterally (for example i.v. in physiological saline solution) or enterally (for example orally, formulated with customary carriers).

Depending on their endogenous stability and their bioavailability, one or more doses of the compounds can be given per day in order to achieve the desired normalisation of the blood glucose values. For example, such a dosage range in humans may be in the range of from about 0.01 mg to 250.0 mg per day, preferably in the range of about 0.01 to 100 mg of compound per kilogram of body weight.

By administering effectors of QC activity to a mammal it could be possible to prevent or alleviate or treat QC-related conditions selected from Mild Cognitive Impairment, Alzheimer's disease, Down Syndrome, Familial Danish Dementia, Familial British Dementia, Huntington's Disease, ulcer disease and gastric cancer with or w/o Helicobacter pylori infections, pathogenic psychotic conditions, schizophrenia, infertility, neoplasia, inflammatory host responses, cancer, psoriasis, rheumatoid arthritis, atherosclerosis, restenosis, lung fibrosis, liver fibrosis, renal fibrosis, graft rejection, acquired immune deficiency syndrome, impaired humoral and cell-mediated immune responses, leukocyte adhesion and migration processes in the endothelium, impaired food intake, sleep-wakefulness, impaired homeostatic regulation of energy metabolism, impaired autonomic function, impaired hormonal balance and impaired regulation of body fluids.

Further, by administering effectors of QC activity to a mammal it could be possible to stimulate gastrointestinal tract cell proliferation, preferably proliferation of gastric mucosal cells, epithelial cells, acute acid secretion and the differentiation of acid producing parietal cells and histamine-secreting enterochromaffin-like cells.

In addition, administration of QC inhibitors to mammals may lead to a loss of sperm cell function thus suppressing male fertility. Thus, the prevent invention provides a method for the regulation and control of male fertility and the use of activity lowering effectors of QC for the preparation of contraceptive medicaments for males.

Furthermore, by administering effectors of QC activity to a mammal it may be possible to suppress the proliferation of myeloid progenitor cells.

The compounds used according to the invention can accordingly be converted in a manner known per se into conventional formulations, such as, for example, tablets, capsules, dragées, pills, suppositories, granules, aerosols, syrups, liquid, solid and cream-like emulsions and suspensions and solutions, using inert, non-toxic, pharmaceutically suitable carriers and additives or solvents. In each of those formulations, the therapeutically effective compounds are preferably present in a concentration of approximately from 0.1 to 80% by weight, more preferably from 1 to 50% by weight, of the total mixture, that is to say, in amounts sufficient for the mentioned dosage latitude to be obtained.

The substances can be used as medicaments in the form of dragées, capsules, bitable capsules, tablets, drops, syrups or also as suppositories or as nasal sprays.

The formulations may be advantageously prepared, for example, by extending the active ingredient with solvents and/or carriers, optionally with the use of emulsifiers and/or dispersants, it being possible, for example, in the case where water is used as diluent, for organic solvents to be optionally used as auxiliary solvents.

Examples of excipients useful in connection with the present invention include: water, non-toxic organic solvents, such as paraffins (for example natural oil fractions), vegetable oils (for example rapeseed oil, groundnut oil, sesame oil), alcohols (for example ethyl alcohol, glycerol), glycols (for example propylene glycol, polyethylene glycol); solid carriers, such as, for example, natural powdered minerals (for example highly dispersed silica, silicates), sugars (for example raw sugar, lactose and dextrose); emulsifiers, such as non-ionic and anionic emulsifiers (for example polyoxyethylene fatty acid esters, polyoxyethylene fatty alcohol ethers, alkylsulphonates and arylsulphonates), dispersants (for example lignin, sulphite liquors, methylcellulose, starch and polyvinylpyrrolidone) and lubricants (for example magnesium stearate, talcum, stearic acid and sodium lauryl sulphate) and optionally flavourings.

Administration may be carried out in the usual manner, preferably enterally or parenterally, especially orally. In the case of enteral administration, tablets may contain in addition to the mentioned carriers further additives such as sodium citrate, calcium carbonate and calcium phosphate, together with various additives, such as starch, preferably potato starch, gelatin and the like. Furthermore, lubricants, such as magnesium stearate, sodium lauryl sulphate and talcum, can be used concomitantly for tabletting. In the case of aqueous suspensions and/or elixirs intended for oral administration, various taste correctives or colourings can be added to the active ingredients in addition to the above-mentioned excipients.

In the case of parenteral administration, solutions of the active ingredients using suitable liquid carriers can be employed. In general, it has been found advantageous to administer, in the case of intravenous administration, amounts of approximately from 0.01 to 2.0 mg/kg, preferably approximately from 0.01 to 1.0 mg/kg, of body weight per day to obtain effective results and, in the case of enteral administration, the dosage is approximately from 0.01 to 2 mg/kg, preferably approximately from 0.01 to 1 mg/kg, of body weight per day.

It may nevertheless be necessary in some cases to deviate from the stated amounts, depending upon the body weight of the experimental animal or the patient or upon the type of administration route, but also on the basis of the species of animal and its individual response to the medicament or the interval at which administration is carried out. Accordingly, it may be sufficient in some cases to use less than the above-mentioned minimum amount, while, in other cases, the mentioned upper limit will have to be exceeded. In cases where relatively large amounts are being administered, it may be advisable to divide those amounts into several single doses over the day. For administration in human medicine, the same dosage latitude is provided. The above remarks apply analogously in that case.

For examples of pharmaceutical formulations, specific reference is made to the examples of WO 2004/098625, pages 50-52, which are incorporated herein by reference in their entirety.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed.

(2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The above disclosure describes the present invention in general. A more complete understanding can be obtained by reference to the following examples. These examples are described solely for purposes of illustration and are not intended to limit the scope of the invention. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure. Although specific terms have been employed herein, such terms are intended in a descriptive sense and not for purposes of limitation.

### Example 1

### Generation of APP695 Transgenic Mice

The aim of this experiment was to generate transgenic mice with neuron-specific over-expression of the human APP695 wild type gene.

### (A) Establishment of Transgenic Plasmid

The plasmid pcDNA3.1-hAPP695wt was used as the template for PCR amplification of the hAPP-wt cDNA with the following primers:

hAPP695-Xhol-F
(SEQ ID NO: 7)
(5′-AATAATCTCGAGGCCACCATGCTGCCCGGTTTGGCACT-3′)
hAPP695-BsrGI-R
(SEQ ID NO: 8)
(5′-ACATATGTACACTAGTTCTGCATCTGCTCAAAG-3′)

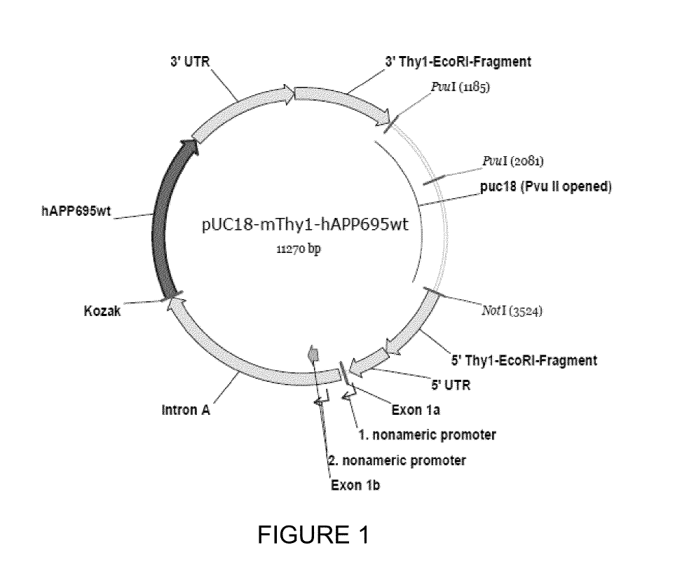

Because the wild type APP695 gene contains an Xho I site, this site was destroyed by a silent point mutation. After that the PCR was conducted, the PCR product was digested with XhoI and BsrGl and ligated with the pUC18-mThy1 vector plasmid. The correct plasmid clone (FIG. 1) was identified by restriction analysis and sequencing.

### (B) Establishment of Screening Strategy

### (i) PCR Screening for First Founder Identification

Forward primer NLE/Q-F:
(SEQ ID NO: 9)
5′-AACTCTTGGCACCTAGAGGATCT-3′
Reverse primer NLE/Q-R:
(SEQ ID NO: 10)
5′-TCCTGGTGTAAGAATTTGCACTT-3′ =
443 by in the start region of hAPP

(ii) qPCR Screening for Founder Confirmation

Forward primer 180F:
(SEQ ID NO: 11)
5′-GCTGTCACCCCAGAGGAG-3′
Reverse primer 184R:
(SEQ ID NO: 12)
5′-CAGAGGAAGGACCTCGACCT-3′
Probe 1p:
(SEQ ID NO: 13)
5′-FAM-CAGCAGAACGGCTACGAAAATCCAACC-TAMRA-3′=
situated in the end region of hAPP

(iii) Head-to-Tail PCR for Testing of transgene Integrity

Forward primer 307F:
(SEQ ID NO: 14)
5′-AGCAAGCCTGGAAGACCTGGGA-3′
Reverse primer 308R:
(SEQ ID NO: 15)
5′-AGACTCAGCCCATCCACTCCTT-3′

### (C) Preparation of the Transgenic Plasmid for Microinjection

The transgenic plasmid pUC18-mThy1-hAPP-wt was linearized with Pvu I and Not I to eliminate plasmid sequences.
Purification of transgenic construct:

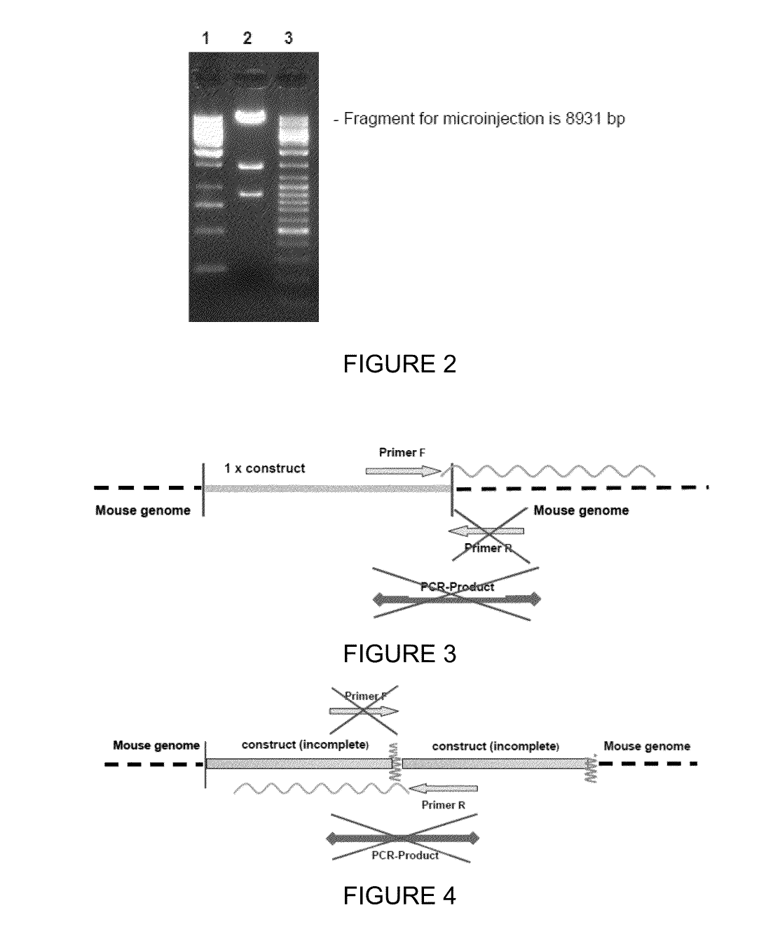

The 8931 by fragment corresponding to the transgenic construct was separated from the vector backbone by agarose gel electrophoresis (FIG. 2 and Table 1) and further purified.

TABLE 1
Lane Description for Agarose Gel shown in Figure 2

| Lane | Sample | Comments |
|---|---|---|
| 1 | Standard: 1 kb ladder | |
| 2 | Restr.: pUC18mThy1- | 8931 bp (fragment for |
| | hAPP695 wt-Not I/Pvu I | injection) + 1443 bp + 896 bp |
| 3 | Standard: DNA ladder mix | |

### (D) Microinjection

### Preparation of DNA:

Control cut for plasmid backbone removal with Not I and Pvu I
   Pronuclear microinjection included:
DNA pronuclear microinjection into (C57BL/6×CBA) F2 oocytes
Re-implantation of minimal 150 viable microinjected oocytes into pseudo-pregnant mice

### (E) Identification of Transgenic Founders

### (i) PCR Screening for Founder Identification

All pups were routinely screened with the primers described under (b)(i) above. The following mice were identified as founders: Fo#8, Fo#12, Fo#26, Fo#31, Fo#35, Fo#37, Fo#38, Fo#39, Fo#40.

All founders could be confirmed by qPCR with the following result:

| | |
|---|---|
| Fo#8, Fo#26, Fo#31, Fo#35, Fo#37, Fo#38, Fo#40 | ct-value between 22-25; |
| Fo#12 und Fo#39 | ct-value at approx. 30. |

### (ii) Head-to-Tail PCR

A head-to-tail PCR was conducted (for primer sequences see (b)(iii) above) to investigate transgenic construct integrity and multiple transgenic copies. The following results were obtained:

| | |
|---|---|
| Fo#8 | strong correct PCR fragment band |
| Fo#12 | negative |
| Fo#26 | strong correct PCR fragment band |
| Fo#31 | very strong correct PCR fragment band |
| Fo#35 | strong correct PCR fragment band |
| Fo#37 | weak correct PCR fragment band |
| Fo#38 | strong correct PCR fragment band |
| Fo#39 | negative |
| Fo#40 | weak correct PCR fragment band |

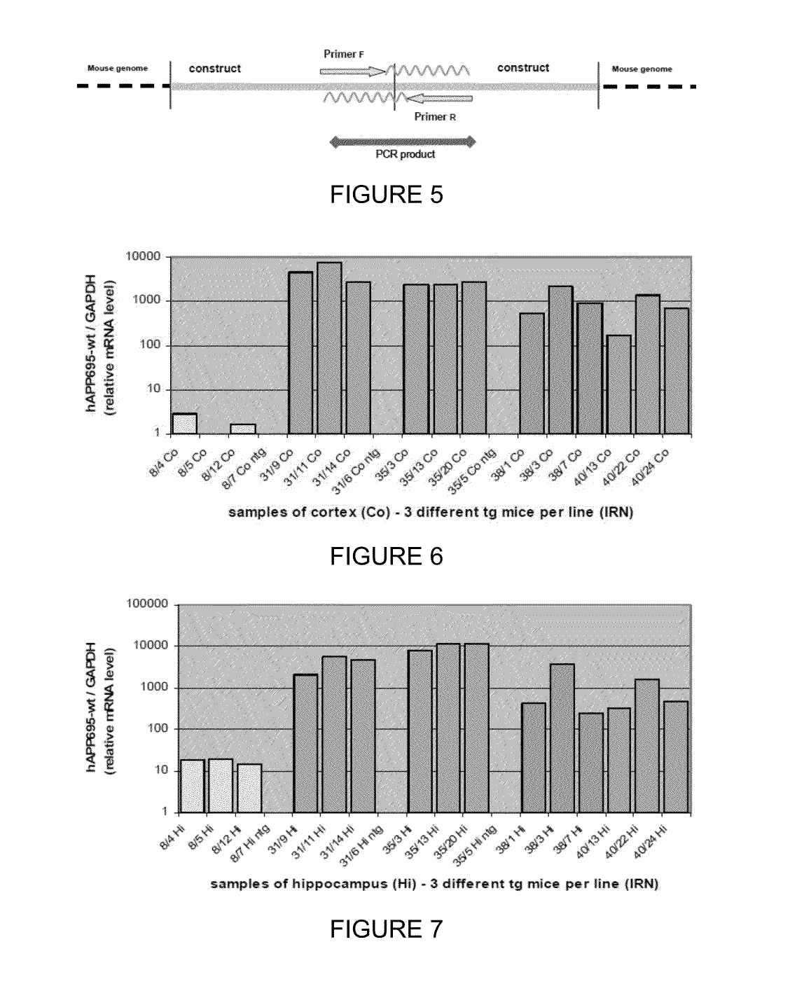

This result leads to the conclusion that all founders with the exception of Fo#12 and Fo#39 have multiple transgenic fragments integrated in tandem direction (as illustrated in FIG. 5).

As more constructs are combined in tandem orientation, as stronger is the head-to-tail PCR band signal.

The founders Fo#12 and Fo#39 appeared to have only a single or incomplete construct integrated (see FIGS. 3 and 4).

### (F) Breeding/Screening of F1 Generation of Founders

Founders Fo#8, Fo#26, Fo#31, Fo#35, Fo#37, Fo#38, and Fo#40 were bred with B6CBA breeding partners.

F1 mice were screened with the above described qPCR primer/probe set.

Transgenic pups could be identified for all founders except Fo#26 who had only non-transgenic pups.

Founder Fo#37 had a very low transgenic level (11 of 60).

(G) mRNA Investigation

Samples of cortex (Co), hippocampus (Hi) and spinal cord (SC) of different transgenic F1 pups of all

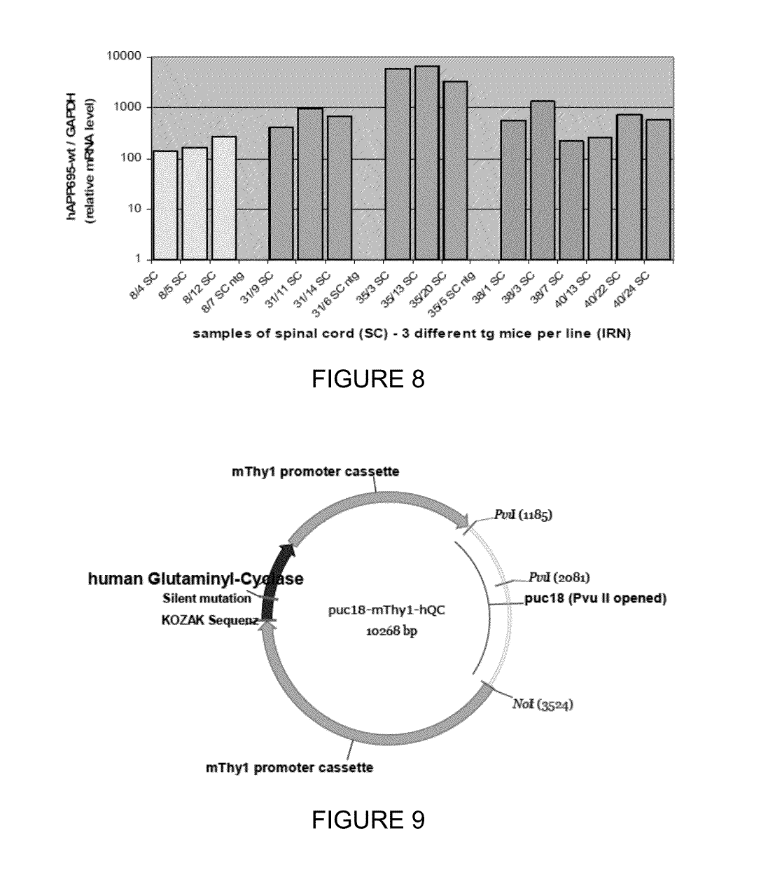

founders (age: 2.5 months) with exception of Fo#26 and Fo#37 were investigated by RT-qPCR together with one non-transgenic control pup. The graph and qPCR raw data are displayed in FIGS. 6-8.

The highest mRNA levels could be detected in Fo#35 and Fo#31 samples.

### Example 2

### Generation of QC Transgenic Mice

The aim of this experimental was to generate transgenic mice with neuron-specific over-expression of the human QC gene.

### (A) Establishment of Transgenic Plasmid

The plasmid pcDNA3.1-hQC was used as template for PCR amplification of the hQC cDNA with the

following primers:

mThy1-hQC-Xhol-F
(SEQ ID NO: 16)
(5′-AAT AAT CTC GAG GCC ACC ATG GCA GGC GGA AGA
CAC CG-3′)
mThy1-hQC-BsrGI-R
(SEQ ID NO: 17)
(5′-ACA TAT GTA CAT TAC AAA TGA AGA TA-3′)

The PCR product was digested with Xhol and BsrGl and ligated with the pUC18-mThy1 vector plasmid (FIG. 9). The correct plasmid clone was identified by restriction and sequencing.

### (B) Establishment of Screening Strategy

(i) qPCR Screening for Founder Identification

Forward primer hQCF1:
(SEQ ID NO: 18)
5′-TCCTACAAGTCTTTGTGTTGGAA-3′
Reverse primer mThy1R1:
(SEQ ID NO: 19)
5′-GAAGGACTTGGGGAGGGAG-3′
Probe 13p:
(SEQ ID NO: 20)
5′-FAM-CAAGTAAGTCGAGGTCCTTCCTCTGCA-TAMRA-3′

### (ii) Head-to-Tail PCR for Testing of Transgene Integrity

Forward primer HTT-mthy1-F:
(SEQ ID NO: 21)
5′-AGCAAGCCTGGAAGACCTGGGA-3′
Reverse primer HTT-mThy1-R:
(SEQ ID NO: 22)
5′-AGACTCAGCCCATCCACTCCTT-3′

### (C) Preparation of the Transgenic Plasmid for Microinjection

The transgenic plasmid pUC18-mThy1-hQC was linearized with Pvu I and Not I to eliminate plasmid sequences.

### Purification of Transgenic Construct:

The 7929 by fragment corresponding to the transgenic construct was separated from the vector

backbone by agarose gel electrophoresis and further purified.

### (D) Microinjection

### Preparation of DNA:

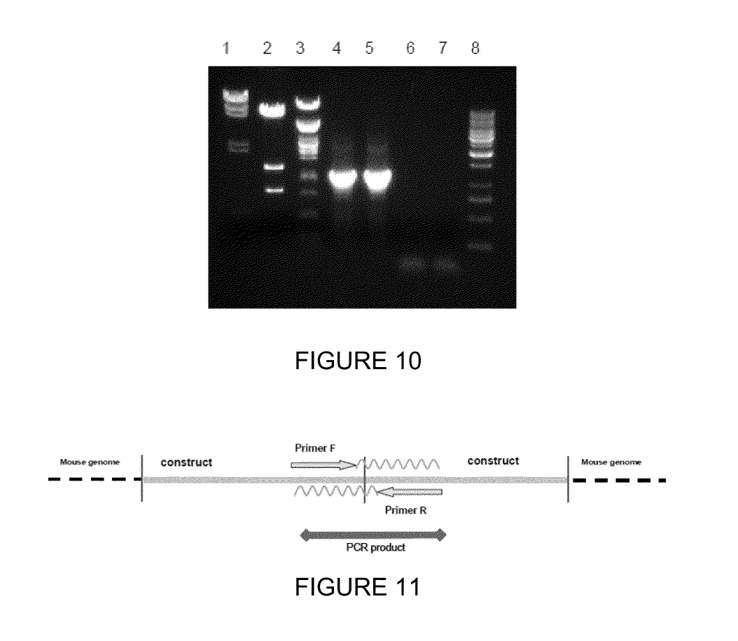

Control cut for plasmid backbone removal with Not I and Pvu I (FIG. 10 and Table 2)
0.8% Agarose/1×TBE
15 μl of 50 μpl PCR-Product/slot
gDNA=mouse genomic DNA
Fragment for microinjection is 7929 by

TABLE 2
Lane Description for Agarose Gel shown in Figure 10

| Lane | Sample |
|---|---|
| 1 | λ Hind III ladder |
| 2 | pUC18-mThy1- |
| | hQC/Not1/PvuI (7929 bp + |
| | 1443 bp + 896 bp) |
| 3 | λ Pst I ladder |
| 4 | PCR: pUC18-mThy1-hQC 27 |
| | ng (1316 bp) |
| 5 | PCR: pUC18-mThy1-hQC 27 |
| | ng + 100 ng gDNA (1316 bp) |
| 6 | PCR: 100 ng gDNA |
| 7 | PCR: Non template control |
| 8 | Standard: 1 kb ladder |

### Pronuclear Microinjection Included:

DNA pronuclear microinjection into (C57BL/6×CBA) F2 oocytes
Re-implantation of minimal 150 viable microinjected oocytes into pseudo-pregnant mice

### (E) Identification of Transgenic Founders

(i) PCR Screening for Founder Identification

All pups were routinely screened with the primers described under (b)(i) above. The following mice were identified as founders:

Fo#37, Fo#38, Fo#43, Fo#48, Fo#53 each of which were born on 30 May 2006, were brown in colour and were all male.

### (ii) Head-to-Tail PCR

A head-to-tail PCR was conducted (for primer sequences see section (b)(ii) above) to investigate transgenic construct integrity and multiple transgenic copies. The following results were obtained:

Fo#37 weak correct PCR fragment band

Fo#38 strong correct PCR fragment band

Fo#43 weak correct PCR fragment band

Fo#48 weak correct PCR fragment band

Fo#53 strong correct PCR fragment band

This result leads to the conclusion that all founders have multiple transgenic fragments integrated in tandem direction (as illustrated in FIG. 11).

The more constructs are combined in tandem orientation, the stronger the head-to-tail PCR band signal.

### (F) Breeding/Screening of F1 Generation of Founders

All founders were bred with B6CBA breeding partners. F1 mice were screened with the above described qPCR primer/probe set.

Transgenic pups could only be identified for Fo#37, Fo#43, and Fo#53. The founder Fo#38 and Fo#48 F1 pups were all non-transgenic.

A list of all animals born, their gender, genotype and use is shown in the appendix.

(G) mRNA Investigation

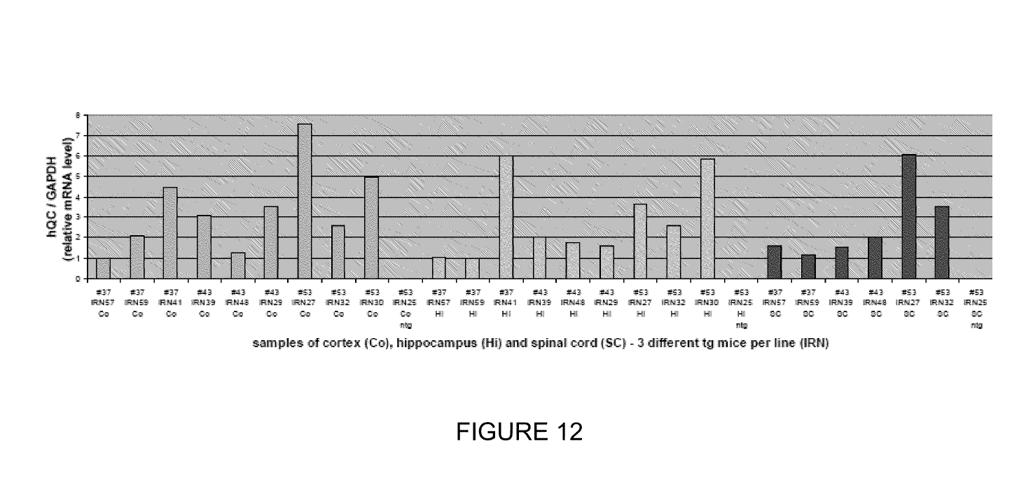

Samples of cortex (Co), hippocampus (Hi) and spinal cord (SC) of different transgenic F1 pups of Fo#37, Fo#43, and Fo#53 (age: 2 to 3.5 months) were investigated by RT-qPCR together with one non-transgenic control pup. The results are shown in FIG. 12.

The highest mRNA levels could be detected in Fo#53 samples.

### Example 3

### Characterisation of APP and QC Transgenic Mice

(A) Genetic constructs

### APP Transgene

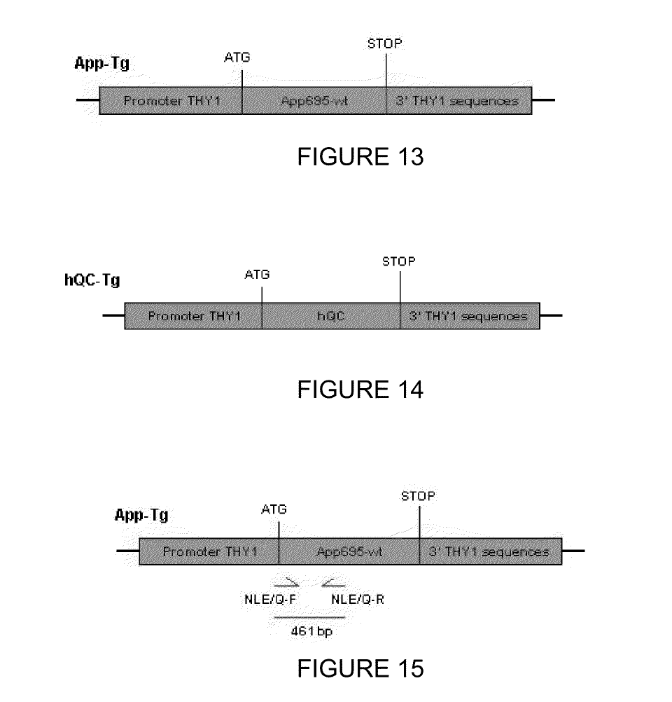

A construct (FIG. 13), which leads to a Thy1 promoter mediated neuronal expression of human Amyloid beta (A4) isoform c precursor protein (NCBI reference sequence: NP_{—}958817.1) was microinjected in fertilized mouse oocytes. Oocytes were transferred to pseudopregnant females and several independent mouse lines were generated:

APPwt−8, −31, −35, −37, −37, −39 and −40.

### (ii) hQC Transgene

A construct (FIG. 14), which leads to a Thy1 promoter mediated neuronal expression of glutaminyl-peptide cyclotransferase precursor protein (NCBI reference sequence: NP_{—}036545.1) was microinjected in fertilized mouse oocytes. Oocytes were transferred to pseudopregnant females and several independent mouse lines were generated: hQC-43, -53 and -63.

### (B) Breeding Performance

(i) Genetic Background hAPP and hQC

The pronucleus injection was conducted in (C57BL/6×CBA) F2 oozytes and subsequently the founders were crossed with the hybrid strain C57BL/6×CBA to yield the F1 generation. Suppliers of original strains: C57BL/6: Harlan
CBA: Harlan

Genetic background of the import strain: 50% C57BL/6, 50% CBA

The imported mice were outcrossed against C57BL/6 (Charles River) to yield a genetic background of 75% C57/BL6, 25% CBA.

(ii) Breeding performance and genotype ratio (Mendelian ratio) hAPPwt-35

Genotyping of 42 litters derived from heterozygous parents revealed an almost normal mendelian ratio (1/2/1) of the different genotypes (wildtype, heterozygous and homozygous). There is no indication of an embryonic or early lethal phenotype originating from the transgene integration neither in heterozygous nor in homozygous animals. The breeding performance is >90%.

TABLE 3
Breeding performance and genotype ratio APPwt-35 mouse line

| Matings APPwt35 | 28 | | | |
|---|---|---|---|---|
| Litter | 42 | | | |
| Total pubs | 308 | | | |
| Average litter size | 7.3 | | | |
| Gender/Genotype | wt | het | hom | Total |
| Male | 37 | 71 | 35 | |
| Female | 31 | 52 | 31 | |
| Total | 68 | 123 | 66 | 257 |
| % | 26 | 48 | 26 | |

(iii) Breeding Performance and Survival of hQC

The breeding performances of hQC-43, -63 and -53 are approximately 85%. Homozygous animals are born and vital in all three lines (hQC-43, -53 and -63) indicating no severe side effect due to the transgene insertion. However, a genotype ratio calculation was not available. One group of hQC-43 animals was observed until senescence of the animals (24 months of age) and no animal died prematurely indicating no adverse effect of hQC overexpression.

TABLE 4
Survival of hQC-43 up to 2 years of age

| hQC-43 | | | |
|---|---|---|---|
| Day of birth | 21.04-28.04. 2008 | | |
| Day of death | 07.04-13.04.2010 | | |
| Gender | Male | | |
| Genotype | wt | het | hom |
| hQC-43 | 6 | 10 | 8 |
| Animals died premature | 0 | 0 | 0 |

### (C) Genotyping

### (i) General Genotyping Assay for the Detection of the APP Transgene

### Assay: PCR+Gel Electrophoresis

This PCR based genotyping assay detects the presence of the transgenic fragment in the mouse genome and allows identification of APP transgene carriers. This assay does not discriminate between heterozygous and homozygous animals (FIG. 15).

### PCR Primer:

NLE/Q-F:
(SEQ ID NO: 23)
AACTCTTGGCACCTAGAGGATCT
NLE/Q-R
(SEQ ID NO: 24)
TCCTGGTGTAAGAATTTGCACTT

### Generated PCR Fragment Size: 449 bp

### PCR Fragment (449 bp):

(SEQ ID NO: 25)
AACTCTTGGCACCTAGAGGATCTCGAGGCCACCATGCTGCCCGGTTTG
GCACTGCTCCTGCTGGCCGCCTGGACGGCTCGGGCGCTGGAGGTACCC
ACTGATGGTAATGCTGGCCTGCTGGCTGAACCCCAGATTGCCATGTTC
TGTGGCAGACTGAACATGCACATGAATGTCCAGAATGGGAAGTGGGAT
TCAGATCCATCAGGGACCAAAACCTGCATTGATACCAAGGAAGGCATC
CTGCAGTATTGCCAAGAAGTCTACCCTGAACTGCAGATCACCAATGTG
GTAGAAGCCAACCAACCAGTGACCATCCAGAACTGGTGCAAGCGGGGC
CGCAAGCAGTGCAAGACCCATCCCCACTTTGTGATTCCCTACCGCTGC
TTAGTTGGTGAGTTTGTAAGTGATGCCCTTCTCGTTCCTGACAAGTGC
AAATTCTTACACCAGGA

### PCR Details

### Reagents:

10×PCR-Buffer¹.: 160 mM (NH₄)₂SO₄
670 mM Tris-HCl
15 mM MgCl₂
0.1% Tween 20

¹Taq buffer from the enzyme supplier.

dNTP-Mix: each nucleotide 25 mM

Taq-Polymerase: 5U/μl

Primer: 10 pmol/μl

PCR-Assay: chrom. DNA: 30-50 ng
10×Puffer 2.5 μl
dNTP-Mix 0.2 μl
Primer1: 0.5 μl
Primer2: 0.5 μl
Taq-Pol.: 0.2 μl
H₂O: to 25 μl

### PCR Conditions:

| | | | |
|---|---|---|---|
| 94.0° C.; 3 min | | | |
| 94.0° C.; 30 sec | 61.0° C.; 30 sec; | 72.0° C.; 90 sec | 2X |
| 94.0° C.; 30 sec | 59.0° C.; 30 sec; | 72.0° C.; 90 sec | 2X |
| 94.0° C.; 30 sec | 57.0° C.; 30 sec; | 72.0° C.; 90 sec | 2X |
| 94.0° C.; 30 sec | 55.0° C.; 30 sec; | 72.0° C.; 90 sec | 28X |
| 94.0° C.; 30 sec | 61.0° C.; 30 sec; | 72.0° C.; 10 min | End |

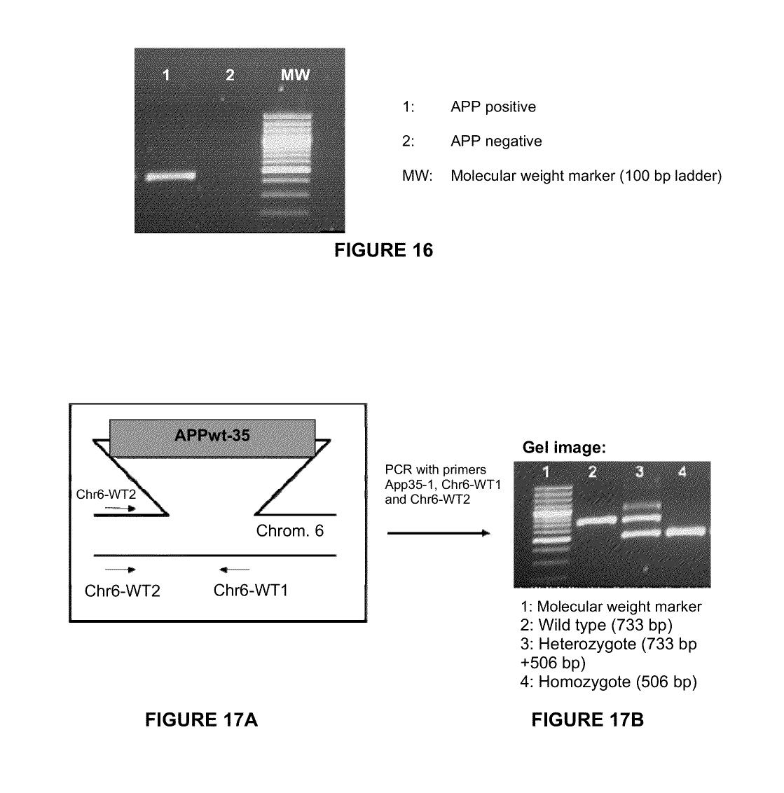

The results of the PCR may be seen in FIG. 16.

### (ii) Specific Genotyping Assay for APPwt-35 Line

### Assay: PCR+Gel Electrophoresis

This genotyping assay for the line APPwt-35 detects the presence of the transgene construct and allows simultaneously the assignment of the zygosity status. The assay is based upon the identification of the integration site of the transgenic fragment into chromosome 6 of line APPwt-35 (see FIG. 17).

### PCR-Primer:

App35-1:
(SEQ ID NO: 26)
TGCCCATATGTCCTAAGCTC
Chr6-WT1:
(SEQ ID NO: 27)
TGGTCATGGCATCTGTTCAC
Chr6-WT2:
(SEQ ID NO: 28)
GGAACAAGATCCTGCGAATG

### Generated PCR Fragments

Wild type allele: 733 bp

Transgenic allele: 506 bp

### Wildtype Fragment (733 bp):

(SEQ ID NO: 29)
GGAACAAGATCCTGCGAATGAATGAGAAGGGAGTCAGATACCTCAGCA
GCTAGTAGTCCCAGTGCCAGGGCAGGAGCCTCCTCCAGGGAGCCGCAG
ACACCAACACATGTATTCCCCGCTTATGCTCAGGATTCTCCCCCTGAA
GTATGTGGAGCTGCAAATCTATGACCGGACGCAGCGCATCCTCCGCGT
TAGGACAGTGACGGAGAAGATCTACTACCTGAGGCTCCACGAGAAACA
TCCACAGGCTGTGTTTCAGTTCTGGATCCGGTTGGTGAAAATTTTACA
GAAAGGTCTGTCCATCACCACCAAAGACCCAAGAATCCAGTTCACTCA
CTGCCTGGTGCCCAAGATGTCCAACTCCTCCACTGAAACAGCAGTAAG
TGCCCCCCCCCCCCCCGGCGGTCTGCGCAGAAATGTATATATCCATGT
ATATACTCATACACTCACATGCACGCACTCGTATGTTCCCAGATGCAA
ATGGACACCTAGCCTTTCATAGATGCCGGTCACTCATCCATAGATTCA
CTCAACACCTTGGATCAGAAATATTCAGGAAACAAACACTGCTCCCTT
CTCACTGTTCCCTGAACAATACAGTGGGATAGTCTATTTACATAGTAT
TGTGTTGTGTGAGGCATGTGCAAAAGCTATGTCATTGCAAATAAGGGA
CTTGATTATAGGGGAGGTGTCTTAGTTAGGGTTTTACTTCTGTGAACA
GATGCCATGACCA

### Transgenic Fragment: (506 bp)

(SEQ ID NO: 30)
GGAACAAGATCCTGCGAATGAATGAGAAGGGAGTCAGATACCTCAGCA
GCTAGTAGTCCCAGTGCCAGGGCAGGAGCCTCCTCCAGGGAGCCGCAG
ACACCAACACATGTATTCCCCGCTTATGCTCAGGATTCTCCCCCTGAA
GTATGTGGAGCTGCAAATCTATGACCGGACGCAGCGCATCCTCCGCGT
TAGGACAGTGACGGAGAAGATCTACTACCTGAGGCTCCACGAGAAACA
TCCACAGGCTGTGTTTCAGTTCTGGATCCGGTTGGTGAAAATTTTACA
GAAAGGTCTGTCCATCACCACCAAAGACCCAAGAATCCAGTTCACTCA
CTGCCTGGTGCCCAAGATGTCCAACTCCTCCACTGAATCGGTGCGGGC
CTCTTCGCTATTACGCCAGGATCAATTCTAGGACTTAAGGTAGGAAAC
TAAGTGGCTGAAGGTAGAGAGAGAATAAGGACAGTGAACGAGTGGGTG
GGTGGGGAGCTTAGGACATATGGGCA

### PCR Details

### Reagents:

10×PCR-Buffer¹.: 160 mM (NH₄)₂SO₄
670 mM Tris-HCl
15 mM MgCl₂
0.1% Tween 20

¹ Taq buffer from the enzyme supplier.

dNTP-Mix: each nucleotide 25 mM

Taq-Polymerase: 5U/μl

Primer: 10 pmol/μl

PCR-Assay: chrom. DNA: 30-50 ng
10×Puffer 2.5 μl
dNTP-Mix 0.2 μl
Primer1: 0.5 μl
Primer2: 0.5 μl
Primer3: 0.5 μl
Taq-Pol.: 0.2 μl
H₂O: to 25 μl

### PCR Conditions:

| | | | |
|---|---|---|---|
| 94.0° C.; 3 min | | | |
| 94.0° C.; 30 sec | 61.0° C.; 30 sec; | 72.0° C.; 90 sec | 2X |
| 94.0° C.; 30 sec | 59.0° C.; 30 sec; | 72.0° C.; 90 sec | 2X |
| 94.0° C.; 30 sec | 57.0° C.; 30 sec; | 72.0° C.; 90 sec | 2X |
| 94.0° C.; 30 sec | 55.0° C.; 30 sec; | 72.0° C.; 90 sec | 28X |
| 94.0° C.; 30 sec | 61.0° C.; 30 sec; | 72.0° C.; 10 min | End |

(iii) General Genotyping Assay for the Detection of the hQC Transgene

### Assay: PCR+Gel Electrophoresis

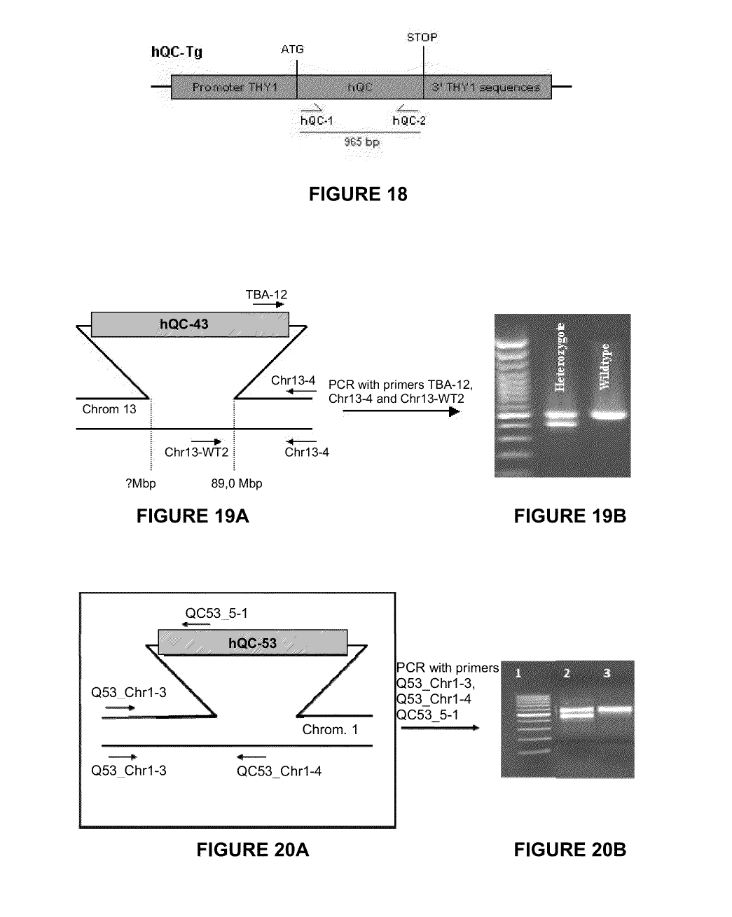

This PCR based genotyping assay detects the presence of the hQC transgenic fragment in the mouse genome and allows identification of hQC transgene carriers. This assay does not discriminate between heterozygous and homozygous animals (see FIG. 18).

### PCR:

Primer1:
hQC-1:
(SEQ ID NO: 31)
GGCCAGAGGAGAAGAATTACC
Primer2:
hQC-2:
(SEQ ID NO: 32)
TTCCAACACAAAGACTTGTAGGA

### Generated PCR Fragment Size: 965 bp

### PCR Fragment (965 bp):

(SEQ ID NO: 33)
GGCCAGAGGAGAAGAATTACCACCAGCCAGCCATTTTGAATTCATCGG
CTCTTCGGCAAATTGCAGAAGGCACCAGTATCTCTGAAATGTGGCAAA
ATGACTTACAGCCATTGCTGATAGAGCGATACCCGGGATCCCCTGGAA
GCTATGCTGCTCGTCAGCACATCATGCAGCGAATTCAGAGGCTCCAGG
CTGACTGGGTCTTGGAAATAGACACCTTCTTGAGTCAGACACCCTATG
GGTACCGGTCTTTCTCAAATATCATCAGCACCCTCAATCCCACTGCTA
AACGACATTTGGTCCTCGCCTGCCACTATGACTCCAAGTATTTTTCCC
ACTGGAACAACAGAGTGTTTGTAGGAGCCACTGATTCAGCCGTGCCAT
GTGCAATGATGTTGGAACTTGCTCGTGCCTTAGACAAGAAACTCCTTT
CCTTAAAGACTGTTTCAGACTCCAAGCCAGATTTGTCACTCCAGCTGA
TCTTCTTTGATGGTGAAGAGGCTTTTCTTCACTGGTCTCCTCAAGATT
CTCTCTATGGGTCTCGACACTTAGCTGCAAAGATGGCATCGACCCCGC
ACCCACCTGGAGCGAGAGGCACCAGCCAACTGCATGGCATGGATTTAT
TGGTCTTATTGGATTTGATTGGAGCTCCAAACCCAACGTTTCCCAATT
TTTTTCCAAACTCAGCCAGGTGGTTCGAAAGACTTCAAGCAATTGAAC
ATGAACTTCATGAATTGGGTTTGCTCAAGGATCACTCTTTGGAGGGGC
GGTATTTCCAGAATTACAGTTATGGAGGTGTGATTCAGGATGACCATA
TTCCATTTTTAAGAAGAGGTGTTCCAGTTCTGCATCTGATACCGTCTC
CTTTCCCTGAAGTCTGGCACACCATGGATGACAATGAAGAAAATTTGG
ATGAATCAACCATTGACAATCTAAACAAAATCCTACAAGTCTTTGTGT
TGGAA

### PCR Details

### Reagents:

10×PCR-Buffer¹.: 160 mM (NH₄)₂SO₄
670 mM Tris-HCl
15mM MgCl₂
0.1% Tween 20

¹ Taq buffer from the enzyme supplier.

dNTP-Mix: each nucleotide 25 mM

Taq-Polymerase: 5U/μl

Primer: 10 pmol/μl

PCR-Assay: chrom. DNA: 30-50 ng
10×Puffer 2.5 μl
dNTP-Mix 0.2 μl
Primer1: 0.5 μl
Primer2: 0.5 μl
Taq-Pol.: 0.2 μl
H₂O: to 25 μl

### PCR Conditions:

| | | | |
|---|---|---|---|
| 94.0° C.; 3 min | | | |
| 94.0° C.; 30 sec | 61.0° C.; 30 sec; | 72.0° C.; 90 sec | 2X |
| 94.0° C.; 30 sec | 59.0° C.; 30 sec; | 72.0° C.; 90 sec | 2X |
| 94.0° C.; 30 sec | 57.0° C.; 30 sec; | 72.0° C.; 90 sec | 2X |
| 94.0° C.; 30 sec | 55.0° C.; 30 sec; | 72.0° C.; 90 sec | 28X |
| 94.0° C.; 30 sec | 61.0° C.; 30 sec; | 72.0° C.; 10 min | End |

(iv) Specific Genotyping assay for hQC-43 Line

### Assay: PCR+Gel Electrophoresis

### Assay Details:

This genotyping assay for the line hQC43 detects the presence of the transgene construct and allows simultaneously the assignment of the zygosity status. The assay is based upon the identification of the integration site of the transgenic fragment into chromosome 13 of line hQC43 (see FIG. 19).

### PCR-Primer:

Primer 1:
TBA-12:
(SEQ ID NO: 34)
TGCCCATATGTCCTAAGCTC
Primer 2:
Chr13-4:
(SEQ ID NO: 35)
GGAGATTGTACGCTAGGAAGG
Primer 3:
Chr13-WT2:
(SEQ ID NO: 36)
AGAAAGCCTTTTTGGCAGTT

### Generated PCR Fragments

Wildtype allele: 517 bp

Transgenic allele: 416 bp

### Wildtype Fragment (517 bp):

(SEQ ID NO: 37)
AGAAAGCCTTTTTGGCAGTTCAGTGTGATGTGTACTGTTGCCTGACTA
CCCATGTTCATCTTAATCCTGCCTGTTTATTGCTGTCTGAGCAAAAGC
AGAACGCATGCTTCTGCATTTCTGAATTCCAACTGTTCTGGACCAATT
TTAGAACTCATCCTTTTCTTTATTCATTAGGACAAACCAGCAACTACT
TATTCCAGGAGAAACTGGTAACATTGTGAGGTTGCTAATTCCTCAACC
TGTCTTACTTGGCACATATTTCATATGATTTGAGGTTGGATTAGTGAT
TTCAAGGGTAGTGCTGAAGGTCAGTATATACAATATGTCTCTGGAAAT
TGTATATTAATTTTCCATGTATGCTGTACAGTTATTTATCAAAATATC
TTTATGTTTAAACCTTGATATCTGAAAATAAGTGAGATGATATTCAAG
TGGCAGAAAAATTGAAGTTTCTTCTACAAATATGAGGAGTTTTTCTAT
TTTAAAATTATAACATCCTTCCTAGCGTACAATCTCC

### Transgenic Fragment: (416 bp)

(SEQ ID NO: 38)
TGCCCATATGTCCTAAGCTCCCCACCCACCCACTCGTTCACTGTCCTT
ATTCTCTCTCTACCTTCAGCCACTTAGTTTCCTACCTTAAGTCCTAGA
ATTGATCCTGGCGTAATAGCGAAGAGGCCCGCACCGATCAACCTGTCT
TACTTGGCACATATTTCATATGATTTGAGGTTGGATTAGTGATTTCAA
GGGTAGTGCTGAAGGTCAGTATATACAATATGTCTCTGGAAATTGTAT
ATTAATTTTCCATGTATGCTGTACAGTTATTTATCAAAATATCTTTAT
GTTTAAACCTTGATATCTGAAAATAAGTGAGATGATATTCAAGTGGCA
GAAAAATTGAAGTTTCTTCTACAAATATGAGGAGTTTTTCTATTTTAA
AATTATAACATCCTTCCTAGCGTACAATCTCC

### PCR Details

### Reagents:

10×PCR-Buffer¹.: 160 mM (NH₄)₂SO₄
670 mM Tris-HCl
15 mM MgCl₂
0.1% Tween 20

¹ Taq buffer from the enzyme supplier.

dNTP-Mix: each nucleotide 25 mM

Taq-Polymerase: 5U/μl

Primer: 10 pmol/μl

PCR-Assay: chrom. DNA: 30-50 ng
10×Puffer 2.5 μl
dNTP-Mix 0.2 μl
Primer1: 0.5 μl
Primer2: 0.5 μl
Primer3: 0.5 μl
Taq-Pol.: 0.2 μl
H₂O: to 25 μl

### PCR Conditions:

| | | | |
|---|---|---|---|
| 94.0° C.; 3 min | | | |
| 94.0° C.; 30 sec | 61.0° C.; 30 sec; | 72.0° C.; 90 sec | 2X |
| 94.0° C.; 30 sec | 59.0° C.; 30 sec; | 72.0° C.; 90 sec | 2X |
| 94.0° C.; 30 sec | 57.0° C.; 30 sec; | 72.0° C.; 90 sec | 2X |
| 94.0° C.; 30 sec | 55.0° C.; 30 sec; | 72.0° C.; 90 sec | 28X |
| 94.0° C.; 30 sec | 61.0° C.; 30 sec; | 72.0° C.; 10 min | End |

(v) Specific Genotyping Assay for hQC-53 Line

### Assay: PCR+Gel Electrophoresis

### Assay Details:

This genotyping assay for the line hQC53 detects the presence of the transgene construct and allows simultaneously the assignment of the zygosity status. The assay is based upon the identification of the integration site of the transgenic fragment into chromosome 1 of line hQC53 (see FIG. 20).

### PCR-Primer:

Primer 1:
QC53_5-1:
(SEQ ID NO: 39)
ACTGAACTCAGGCTGTCAGG
Primer 2:
QC53_Chr1-3:
(SEQ ID NO: 40)
CAGGAGGAATCTGGTCAATG
Primer 3:
QC53_Chr1-4:
(SEQ ID NO: 41)
AGCAGAGACCAAGGAGGATT

### Generated PCR Fragments

Wild type allele: 552 bp

Transgenic allele: 419 bp

### Wild Type Fragment (552 bp):

(SEQ ID NO: 42)
CAGGAGGAATCTGGTCAATGCTCCCCCAGCAATGGTTAATGCCCACTC
ATCCAAGGATGAGAAGGTCATTTGATCACCTCAGTTAAGTGTGGCCTT
ATTAAATTTAATTCAAAGGGGGAGACCATACCGTGAGAAAGTATGCCT
TTCACAGCTTCCCACTCTAACAAGCCAAATGGTCTTGTGCTAAGGAGC
GGGTCATCACCCCCTCCTCCCTATTCCCTTTCTGGCACCTGAGGCTAT
AAAAAGCTAAATTATAGACCCCTCTTCCTTATCTCTTCCTGACTCCCA
AGACTTCTAAGGACATGAGTTATGTGCTGAGCCCAGCCTGACTCCCAA
GGCTGTTAAGGAGGATTCTACATCCTGGATATAGATTCAGAGTGCCTC
CCGCCTGCAGTCTGAGGTCGGCCTTCATGTCCCCAGATGCCCACTTCT
TTGTTCTTTGTTAATTCCCCCTCAACCCCTCCCTATTTCCCTTGCTGT
GTGCTTAAAACCTGGCGTTTCAGCCTAATAAACTGAGTGAGACCTTGA
CAGGAATCCTCCTTGGTCTCTGCT

### Transgenic Fragment: (419 bp)

(SEQ ID NO: 43)
CAGGAGGAATCTGGTCAATGCTCCCCCAGCAATGGTTAATGCCCACTC
ATCCAAGGATGAGAAGGTCATTTGATCACCTCAGTTAAGTGTGGCCTT
ATTAAATTTAATTCAAAGGGGGAGACCATACCGTGAGAAAGTATGCCT
TTCACAGCTTCCCACTCTAACAAGCCAAATGGTCTTGTGCTAAGGAGC
GGGTCATCACCCCCTCCTCCCTATTCCCTTTCTGGCACCTGAGGCTAT
AAAAAGCTAAATTATAGACCCCTCTTCCTTATCTCTTCCTGACTCCCA
AGATCCCCGGGCGAGCTCGAATTCAGAGACCGGGAACCAAACTAGCCT
TTAAAAAACATAAGTACAGGAGCCAGCAAGATGGCTCAGTGGGTAAAG
GTGCCTACCAGCAAGCCTGACAGCCTGAGTTCAGT

### PCR Details

### Reagents:

10×PCR-Buffer¹.: 160 mM (NH₄)₂SO₄
670 mM Tris-HCl
15mM MgCl₂
0,1% Tween 20

¹ Taq buffer from the enzyme supplier.

dNTP-Mix: each nucleotide 25 mM

Taq-Polymerase: 5U/μl

Primer: 10 pmol/μl

PCR-Assay: chrom. DNA: 30-50 ng
10×Puffer 2.5 μl
dNTP-Mix 0.2 μl
Primer1: 0.5 μl
Primer2: 0.5 μl
Primer3: 0.5 μl
Taq-Pol.: 0,2 μl
H₂O: to 25 μl

### PCR Conditions:

| | | | |
|---|---|---|---|
| 94.0° C.; 3 min | | | |
| 94.0° C.; 30 sec | 61.0° C.; 30 sec; | 72.0° C.; 90 sec | 2X |
| 94.0° C.; 30 sec | 59.0° C.; 30 sec; | 72.0° C.; 90 sec | 2X |
| 94.0° C.; 30 sec | 57.0° C.; 30 sec; | 72.0° C.; 90 sec | 2X |
| 94.0° C.; 30 sec | 55.0° C.; 30 sec; | 72.0° C.; 90 sec | 28X |
| 94.0° C.; 30 sec | 61.0° C.; 30 sec; | 72.0° C.; 10 min | End |

(vi) Specific Genotyping Assay for hQC-63 Line

### Assay: PCR+Gel Electrophoresis

### Assay Details:

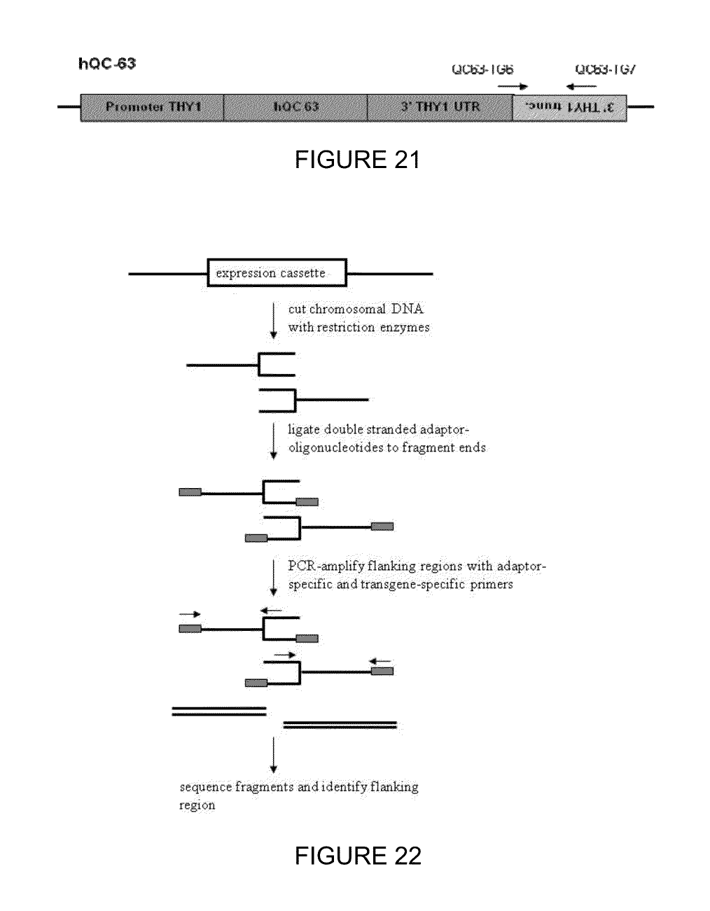

This genotyping assay detects a specific rearrangement of the expression cassette in line hQC63, which occurred during chromosomal integration of transgene construct. The genotyping assay detects the presence of the hQC63 expression cassette but does not allow discrimination of heterozygous and homozygous animals. A schematic view of the primer binding sites is shown in FIG. 21.

### PCR:

Primer1:
hQC63-TG6:
(SEQ ID NO: 44)
CAGGGACTTTGGTGCATAAG
Primer2:
hQC63-TG7:
(SEQ ID NO: 45)
ATTGATCCTGGCGTAATAGC

Generated PCR Fragment iSze: 580 bp

### PCR Fragment (580 bp):

(SEQ ID NO: 46)
CAGGGACTTTGGTGCATAAGTATGTACCATGCCCTTTTTTCACAGTCC
TAGCTCTGCAGAAGTGCAGCCTGAAGGCCTGTCTGCTGAGAGGACATG
CCCTGGAGCCCTGAAACAGGCACAGTGGGAGGAGGAACGGAGGATGAC
AGGCATCAGGCCCTCAGTCCAAAAGCAACCACTTGAGAATGGGCTGGA
GTACGAAACATGGGGTCCCGTCCCTGGATCCCTCCTCAAAGAGTAATA
AGTAAAATATAAACAGGTACCCCAGGCCGTTCTGGGTTTGGGTTGTAA
TGGGATCCATTTGCAGAGAACTATTGAGACAGCCCAGCCGTACTGTGA
CAGGCAATGTGGGGGAGGAGGTTGAATCACTTGGTATTTAGCATGAAT
AGAATAATTCCCTGAACATTTTTCTTAAACATCCATATCTAAATTACC
ACCACTCGCTCCCAGTCTTCCTGCCTTTGCGCCAGCCTCCTGTCTGGC
CATGCCTGAAGAAGGCTGGAGAAGCCACCCACCTCAGGCCATGACACT
GCCAGCCACTTGGCAGGTGCAGCCAAACCTGAGCTATTACGCCAGGAT
CAAT

### PCR Details

### Reagents:

10×PCR-Buffer¹.: 160 mM (NH₄)₂SO₄
670 mM Tris-HCl
15 mM MgCl₂
0.1% Tween 20

¹ Taq buffer from the enzyme supplier.

dNTP-Mix: each nucleotide 25 mM

Taq-Polymerase: 5U/μl

Primer: 10 pmol/μl
PCR-Assay: chrom. DNA: 30-50 ng
10×Puffer 2.5 μl
dNTP-Mix 0.2 μl
Primer1: 0.5 μl
Primer2: 0.5 μl
Taq-Pol.: 0.2 μl
H₂O: to 25 μl

### PCR Conditions:

| | | | |
|---|---|---|---|
| 94.0° C.; 3 min | | | |
| 94.0° C.; 30 sec | 61.0° C.; 30 sec; | 72.0° C.; 90 sec | 2X |
| 94.0° C.; 30 sec | 59.0° C.; 30 sec; | 72.0° C.; 90 sec | 2X |
| 94.0° C.; 30 sec | 57.0° C.; 30 sec; | 72.0° C.; 90 sec | 2X |
| 94.0° C.; 30 sec | 55.0° C.; 30 sec; | 72.0° C.; 90 sec | 28X |
| 94.0° C.; 30 sec | 61.0° C.; 30 sec; | 72.0° C.; 10 min | End |

### (D) Identification of the Transgene Integration Site

A PCR-based approach was used for the identification of the chromosomal integration site of the transgenic fragment. In this approach double-stranded adaptor oligonucleotides were ligated to the ends of DNA fragments derived from restriction enzyme digests of the chromosomal DNA from carrier animals followed by two rounds of nested PCR using adaptor-specific and transgene-specific primers. The generated fragments are separated by agarose gel electrophoresis, eluted from the gel matrix and sequenced (see FIG. 22).

(i) hAPP Transgene Integration Sites

Integration mapping allowed the identification of the 3-prime integration site of the APP transgene fragment in line APPwt-35 on Chromosome 6 (map position 29 236 121 bp; NCBI37/mm9 assembly). The precise localisation of the 5′-integration site is still unknown.

Sequence of the APPwt-35 transgene insertion site:

(SEQ ID NO: 47)
ACGCAGCGCATCCTCCGCGTTAGGACAGTGACGGAGAAGATCTACTACC
TGAGGCTCCACGAGAAACATCCACAGGCTGTGTTTCAGTTCTGGATCCG
GTTGGTGAAAATTTTACAGAAAGGTCTGTCCATCACCACCAAAGACCCA
AGAATCCAGTTCACTCACTGCCTGGTGCCCAAGATGTCCAACTCCTCCA
CTGAATCGGTGCGGGCCTCTTCGCTATTACGCCAGGATCAATTCTAGGA
CTTAAGGTAGGAAACTAAGTGGCTGAAGGTAGAGAGAGAATAAGGACAG
TGAACGAGTGGGTGGGTGGGGAGCTTAGGACATATGGGCAGGAGTCCCA
GGTCTTCCAGGCTTGCTGACTTGGCCAGAGGGACAGATGGGTGTCATGG
CCAGCTGC
Bold: Chromosome 6 sequence
Underlined: 3′-end transgene expression cassette

### (ii) hQC Transgene Integration Sites

hQC-43 Transgene Integration Site

In line hQC-43, the 3′-integration site of the expression cassette was identified on chromosome 13 (map position 89 014 418 bp; NCBI37/mm9 assembly). The precise localisation of the 5′-integration site for line hQC-43 is unknown.

Sequence of the hQC43 3′-transgene insertion site:

(SEQ ID NO: 48)
GCAGCTGGCCATGACACCCATCTGTCCCTCTGGCCAAGTCAGCAAGCC
TGGAAGACCTGGGACTCCTGCCCATATGTCCTAAGCTCCCCACCCACC
CACTCGTTCACTGTCCTTATTCTCTCTCTACCTTCAGCCACTTAGTTT
CCTACCTTAAGTCCTAGAATTGATCCTGGCGTAATAGCGAAGAGGCCC
GCACCGATCAACCTGTCTTACTTGGCACATATTTCATATGATTTGAGG
TTGGATTAGTGATTTCAAGGGTAGTGCTGAAGGTCAGTATATACAATA
TGTCTCTGGAAATTGTATATTAATTTTCCATGTATGCTGTACAGTTAT
TTATCAAAATATCTTTATGTTTAAACCTTGATATCTGAAAATAAGTGA
GATGATATTCAAGTGG
Bold: Chromosome 13 sequence
Underlined: 3′end transgene expression cassette

In order to characterize the 5′-integration site an ordered set of primers were designed which bind to chromosome 13 regions with increasing distance upstream of the 3′-integration site. PCR reactions using homo- and heterozygous DNA as templates showed that the transgene integration deleted a DNA region of about 50 kb upstream of the integration site.

The chromosomal integration of the transgene cassette occurred in intron 1 of the mouse Edil3 gene (EGF-like repeats and discoidin I-like domains 3) and the 50 kb upstream deletion removed the first coding exon of Edil3. Hence, homozygous hQC43 animals are devoid of Edil3 gene function.

hQC-53 Transgene Integration Site

In line hQC-53, the 5′-integration site of the expression cassette was mapped on chromosome 1 (map position 118 168 889 bp; NCBI37/mm9 assembly). The 3′-integration site for line hQC-53 is still unknown.

Sequence of the hQC53 5′-transgene insertion site:

(SEQ ID NO: 49)
GCCTTATTAAATTTAATTCAAAGGGGGAGACCATACCGTGAGAAAGTA
TGCCTTTCACAGCTTCCCACTCTAACAAGCCAAATGGTCTTGTGCTAA
GGAGCGGGTCATCACCCCCTCCTCCCTATTCCCTTTCTGGCACCTGAG
GCTATAAAAAGCTAAATTATAGACCCCTCTTCCTTATCTCTTCCTGAC
TCCCAAGATCCCCGGGCGAGCTCGAATTCAGAGACCGGGAACCAAACT
AGCCTTTAAAAAACATAAGTACAGGAGCCAGCAAGATGGCTCAGTGGG
TAAAGGTGCCTACCAGCAAGCCTGACAGCCTGAGTTCAGTCCCCACGA
ACTACGTGGTAGGAGAGGACCAACCAACTCTGGAAATCTGTTCTGCAA
ACACATGCTCACACAC
Bold: Chromosome 1 sequence
Underlined: 5′end transgene expression cassette

The chromosomal integration of the hQC53 transgene cassette occurred in intron 12 of the mouse Cntnap5a gene (contactin associated protein-like 5A). Hence, ctnap5a gene function might be impaired in hQC53-transgenic animals.

hQC-63 Transgene Integration Site

Breeding of hQC-53 founder animals with wild types revealed that the founder carried multiple transgene insertions at different chromosomal loci which separated upon breeding. Therefore, offspring with an insertion pattern different from the above hQC-53 pattern were treated as a separate line and named hQC-63.

Despite numerous mapping approaches an unequivocal determination of the transgene insertion region was not possible for line hQC-63.

### (E) Transgene Expression Analysis (RNA, Protein, IHC)

(i) Transgene Expression of hAPP: APP ELISA of Several APP Founder Lines (App-wt-31,35, 37 and 40)

From each line 3-10 transgenic animals were killed and brains dissected. The cerebrum and cerebellum were prepared separately and processed for ELISA detection of human APP.

Brain samples were weighed and transferred into a bead mill (Precellys) tube, containing 1000 μl of 2% SDS and protease inhibitors (complete mini, Roche, Kat.Nr: 1836153 supplemented with 10 μl 1M AEBSF (ROTH/Karlsruhe) per 10 ml of 2% SDS solution). Homogenization was achieved using the homogenizer at 6500rpm for 30s. Afterwards, the homogenate was removed and the tube was washed with the extraction buffer (10 times the volume of the brain weight). The combined homogenate was transferred into another conical tube and subjected to sonification. The remaining cell debris were pelleted by centrifugation at 13.000 g, 4° C., for 15 min. the supernatant was removed and subjected to Western-Blot or ELISA analysis. Alternatively, it was stored at −80° C.

For determination of the APP-concentration, the samples were diluted using EIA buffer (1:10), which is supplied with the ELISA kit (IBL; cat-No. JP27731). The ELISA was performed according to the recommendations of the manufacturer.

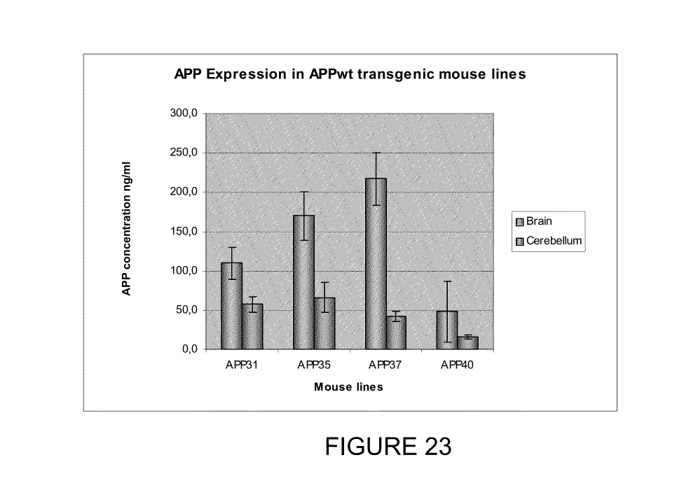

FIG. 23 shows a strong expression of human APP in the lines APP-31, -35 and -37 and a weak expression in mouse line APP-40.

(ii) hAPP Western Analysis of Heterozygous Transgenenic APP Mice (Age 6,5-8 months).

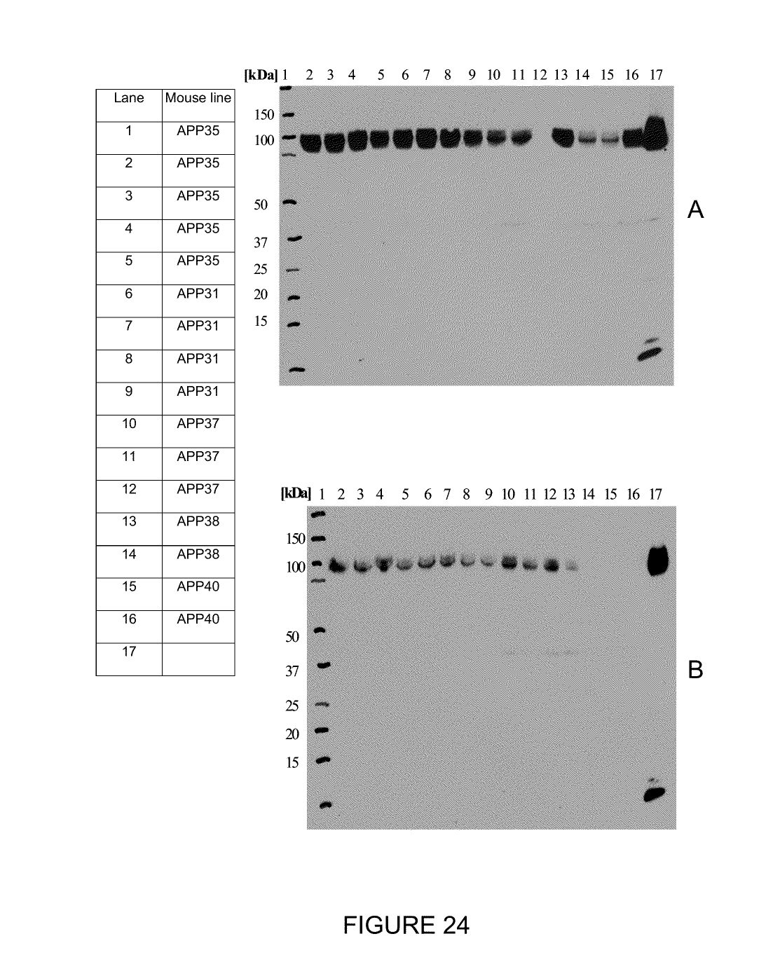

Animals were killed and perfused for 2 min with PBS buffer. After preparation and dissection of cerebrum and cerebellum the brain samples were frozen in liquid nitrogen and stored at −80° C. for further analysis. Brain samples were homogenized in Precelys 24 tubes (containing 2% SDS (per 100 mg brain 1000 μl 2% SDS). The homogenized samples were sonified for 30 sec and centrifuged for 15 min 13.000 rpm. The pellets were dissolved in 5×sample buffer for electrophoresis (see FIG. 24).

### (F) Characterization of hQC Transgene Expression

### (i) Comparison of Transgene Expression Levels in the hQC Transgenic Lines

Quantitative RT-PCR on total brain RNA from heterozygous animals (n=3 for lines hQC43 and 53; n=4 for hQC63) was used to compare the transgene expression levels in the various hQC transgenic lines.

The relative quantification method with β-actin expression as endogenous reference was used for the calculation of the transgene expression rates and the values were normalized against transgene expression in hQC53 heterozygotes.

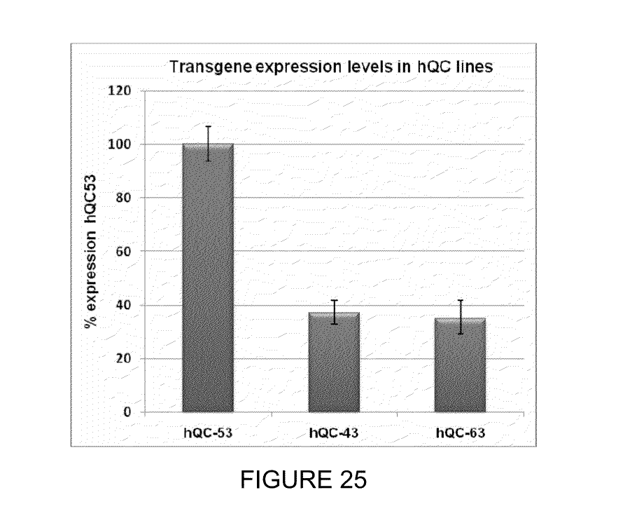

The results showed that transgene expression is highest in line hQC53 with line hQC 43 and hQC63 reaching about 35% of the hQC53 expression level (FIG. 25).

### (ii) Stability of hQC Transgene Expression During Lifespan

Analysis of age-dependent phenotypes using transgenic animal models requires a stable and uniform transgene expression during the animal lifespan. Quantitative RT-PCR on total brain RNA was used to compare the transgene expression rate in two months old hQC43 heterozygotes (n=3) with expression rates in 24 months old hQC43 homo- (n=3) and heterozygotes (n=3).

The relative quantification method with β-actin expression as endogenous reference was used for the calculation of the transgene expression rates and normalized against transgene expression in hQC43 2 months old heterozygotes.

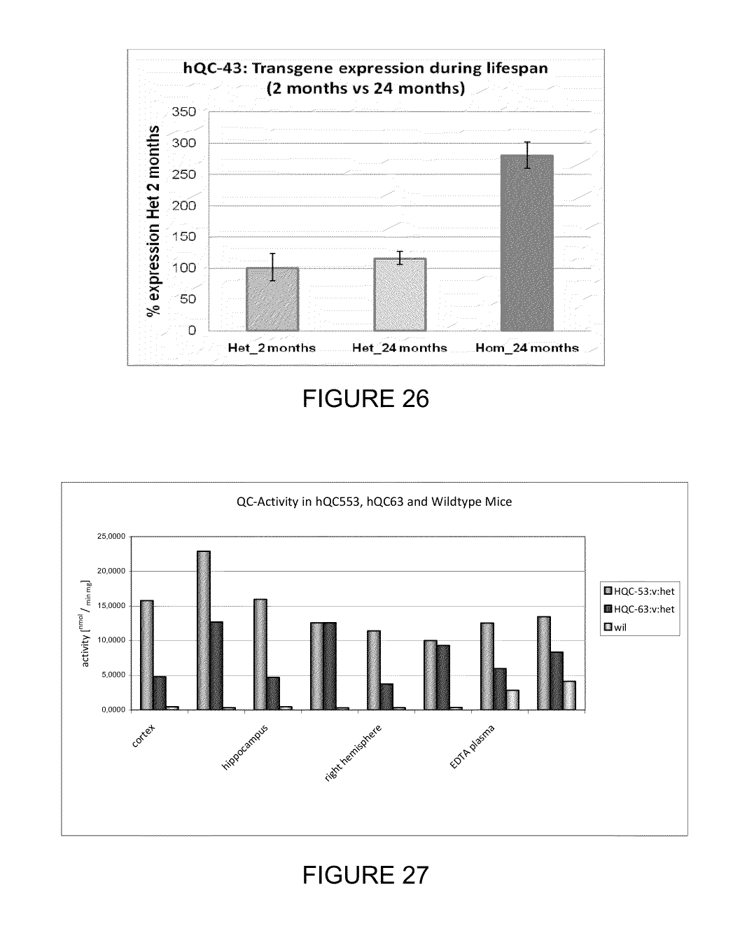

The results showed that hQC43 transgene expression in 24 months old animals is unaltered in comparison to the expression rate in 2 months old animals. Hence, transgene expression is stable during the lifespan of the hQC43 transgenic animal model (see FIG. 26).

### (G) HQC: Enzyme Activity

The brain samples of human QC-transgenic mice were homogenized using a QC extraction buffer consisting of 10 mM Tris, pH 7.5; 100 mM NaCl; 5 mM EDTA; 0.5% (^{v}/ᵥ) Triton; 10% (^{v}/ᵥ) glycerol and 1 tablet of complete Mini (Roche, Germany) per 7 ml. For QC extraction, 10 μl of extraction buffer were added to 1 mg of tissue. Homogenization was carried out using a precellys 24 homogenizer (peqlab, Germany) (6,500 rpm, 2 times 30 s with 10 s break) in 2 ml homogenization tubes with Ø1.4 mm ceramic spheres (peqlab, Germany). Tubes were stored on ice and centrifuged at 7,000×g for 10 min (4° C.) to separate spheres and remove foam. The tissue pellet was resolved and the solution was transferred to new reaction tubes for the following sonification (9 cycles in 10 s at 70% amplitude; sonopuls, bandelin, Germany). Afterwards, the sample solution was centrifuged at 13,000×g for 30 min (4° C.). The supernatant was diluted 1:5 and protein concentration was determined (Bradford reagent).

The QC activity was determined applying an HPLC-assay essentially as described elsewhere (Cynis et al., BBA, 2006) since continuous assay methods for QC activity are hampered by aminopeptidase activities in the crude extracts. The assay is based on conversion of H-Gln-βNA to pGlu-βNA. The sample consisted of 50 μM H-Gln-βNA in 25 mM MOPS, pH 7.0, 0.1mM N-Ethylmaleinimide (NEM) and enzyme solution in a final volume of 1 ml. Substrate and NEM were pre-incubated for 15 min at 30° C. The sample was centrifuged at 16.000×g, 4° C., for 20 min. The reaction was started by addition of 100 μl sample. The reaction mix was further incubated at 30° C. and constantly shaken at 300 rpm in a thermomixer (Eppendorf). Test samples were removed at time points of 0, 5, 10, 15, 22, 30 and 45 min. The reaction was immediately stopped by boiling for 4 min. Test samples were cooled on ice and stored at −20° C. For analysis samples were thawed on ice and centrifuged at 4° C. for 20 min at 16,000×g. All HPLC measurements were performed using a RP18 LiChroCART HPLC-Cartridge [LiChroCART 125-4, LiChrospher 100, RP-18e (5 μm)] and the HPLC system D-7000 (Merck-Hitachi). Briefly, 20μl of the sample were injected and separated by increasing concentration of solvent A (acetonitrile containing 0.1% TFA) from 8% to 20% in solvent B (H₂O containing 0.1% TFA) in 8 min at a flowrate of 1^{ml}/min. QC activity was quantified from a standard curve of pGlu-βNA (Bachem, Switzerland) determined under assay conditions.

The results of this study may be seen in FIG. 27. The data in this Figure clearly suggest a significant increase of QC activity in the transgenic animals compared to the wild type. According to these results, line hQC-53 expresses the QC most efficiently.

### (H) Histology APP

Immunohistochemistry was performed on paraffin brain sections of APPwt-35 mice to evaluate the APP expression.

The following antibodies were used on coronal sections of APPwt-35 brains:
6E10 x-4x-Aβ-specific antibody (mouse monoclonal, Calbiochem, Merck, Darmstadt, Germany; dilution 1:30000)
APP C-terminal-specific antibody (rabbit polyclonal, 127003, Synaptic systems, 1:100000)

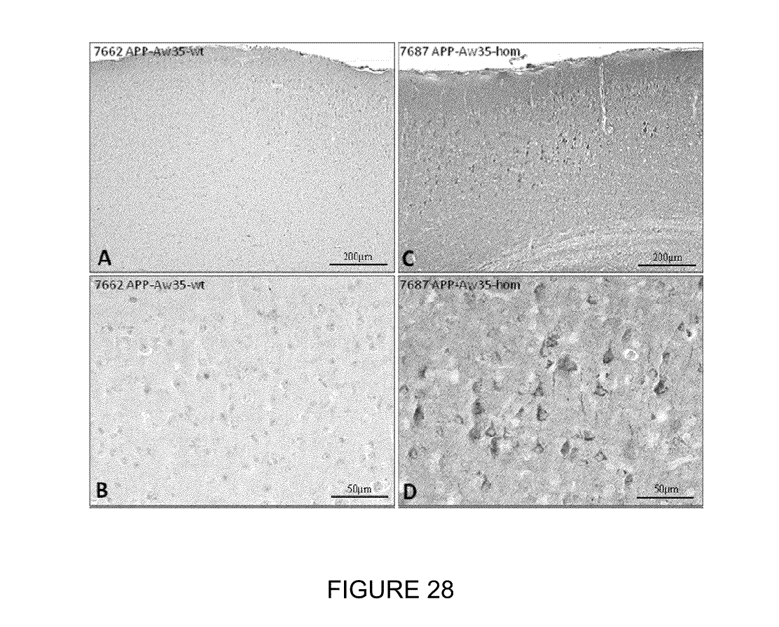

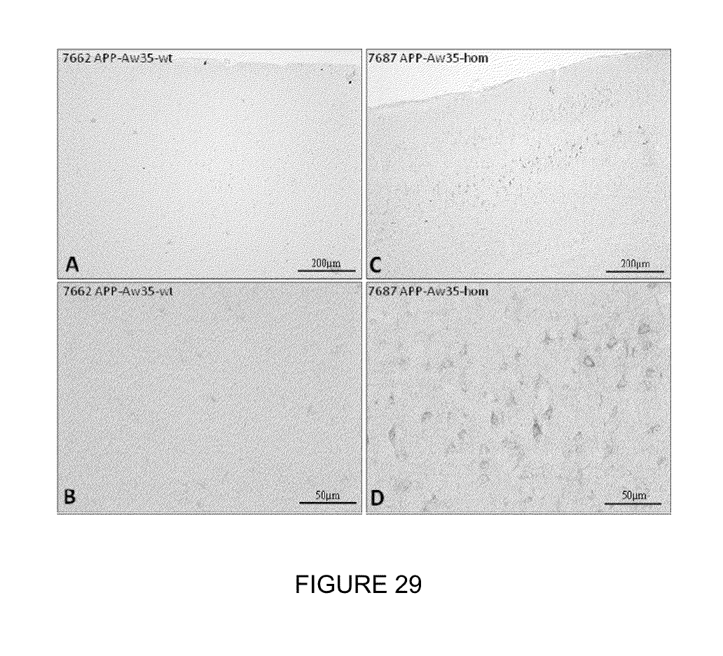

Homozygous and wildtype animals of APPwt-35 at the age of 4 months were devoid of plaques and extracellular amyloid depositions. Nevertheless the overexpression of APP is verified by intraneuronal and diffuse extracellular APP in the APPwt-35 -hom animals. The Amyloid Precursor Protein is immunoreactive to both the APP C-terminal antibody and the 6E10 antibody; both stainings reveal a prominent difference between APPwt-35-wt and APPwt-35-hom (see FIGS. 28 and 29).

(i) Histology hQC-43

### Methods:

The brains of 7 month old animals (hQC-43) were perfused with washing buffer (PBS), fixed with 4% PFA, and cryoprotected in 30% sucrose. The brains were cut into sample pieces and snap-frozen at −68° C. with n-hexane. 30 μm coronal sections were stained free floating using the two step DAB method. As primary antibody the human QC-specific antibody hQC8696 (rabbit, polyclonal, Probiodrug) diluted 1:50 000 was used. As secondary antibody a biotinylated goat-anti-rabbit antibody (Vector) diluted 1.1 000 was used with an avidin-biotin-complex kit (Vactastain, Vector), visualizing the immunoreactivity with an peroxidase subtrate kit (ImmPact DAB, Vector) according to the manufacturer's instructions.

### Results:

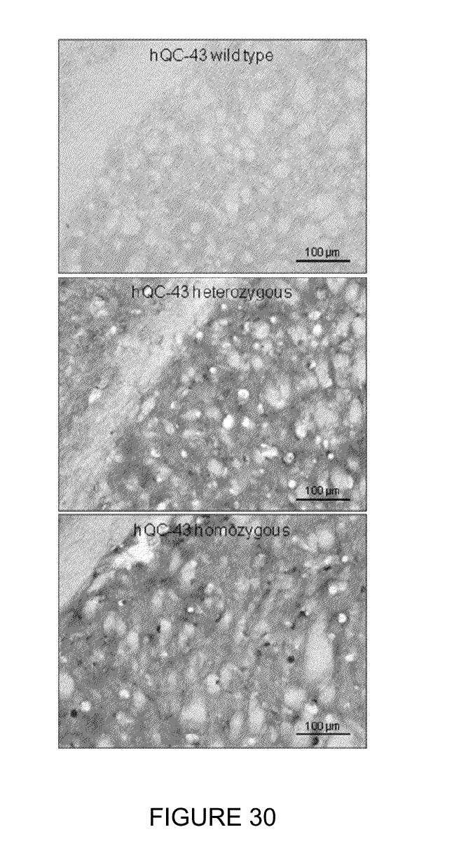

The brains of animals overexpressing hQC showed an increased immunoreactivity in all stained sections (forebrain, midbrain, cerebellum and brainstem). Heterozygous animals show immunoreactivity of the neuropile and single cells, which is increased in homozygous animals (more cells and darker staining) compared to wild type animals. The results are shown in FIG. 30 where it can be seen that a gene-dose dependent increase of intracellular immunoreactivity can be observed in single neurons, but also the neuropiles of heterozygous and homozygous animals show increased immunoreactivity.

### (I) Behavioral Phenotyping

### Methods:

### Primary Screen

The primary screen is used to prompt animals' general health, neurological reflexes and sensory functions, that could interfere with further behavioral assays. It consists of 15 short tests and is based on the guidelines of the SHIRPA protocol, which provides a behavioral and functional profile by observational assessment.

### Pole Test

The Pole test is a simple test to detect motor-coordinative deficits. Animals are μlaced head-up directly under the top end of a vertical metal pole and time to orientate themselves down (t-turn) is measured. Aberrant activities (e.g. falling, jumping, sliding) are recorded as 120 s (cutoff-time). The best performance over five trials is used for analysis.

### Rotarod

The Rotarod paradigm is a common test of motor function, where mice must continuously walk forward on a rotating rod to keep from falling off. The latencies to fall of the accelerating rod (4 to 40 rpm over a five minute period) measured in nine test trials serve as index for motor balance and coordination, as well as for motor learning.

### Constant Hotplate

Acute thermal pain sensitivity is investigated on the 52.5° C. warm surface of a constant hotplate. Hind paw withdrawal latency (or shaking/licking of the hind paw) is measured twice: first without former habituation and then after habituation on a 32.0° C. hotplate. Cutoff-time is 60 seconds.

### Tail Flick

The tail flick is a spinal reflex in which the mouse moves its tail out of the path of a thermal stimulus directed to the tip of the tail. This tail withdrawal latency is measured three times with at least 60 minutes inter trial intervals.

### Exploratory Behavior

Within a pilot experiment exploratory behavior was investigated with help of an operant wall system installed within a type 3 cage. During 75 minutes of free exploration number of nose pokes was measured by automated counting of light beam breaks in two holes.

(i) hAPP

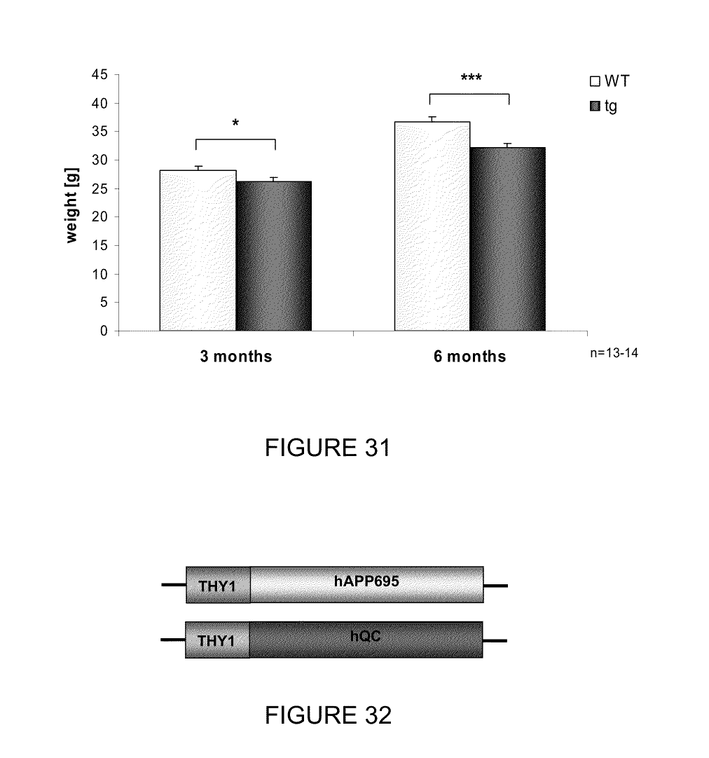

In the APP-wt-31 mouse line two primary screens were performed with a male testing group (transgene vs. wild type) at the age of 3 and 6 months. In both screens no neurological or dysmorphological abnormalities were detected which could be correlated with a specific genotype. But weight was significantly lower in transgene animals compared to wild type controls (FIG. 31).

(ii) hQC

Primary screen analysis in young hQC-43 animals (males and females) aged 3 months did not reveal neurological or dysmorphological abnormalities in homozygous mice. Only weight was significantly reduced in the male HOM group compared to wildtype controls (t-test p<0,05).

Adult hQC-43 females (9 months) were tested in a variety of assays, detecting comparable motor performances of HOM, HET and WT mice on the pole and no significant differences but a slightly improved performance of HOM on the accelerating rotarod, as well as normal pain sensitivity on the constant hotplate and in the tail flick assay. Nose poke activity measured in an experiment for exploratory behavior was slightly increased in HOM mice. In aged hQC-43 males and females (21 months) primary screen analysis again could not detect distinct abnormalities in HOM and HET animals. But weight was slightly decreased in HOM females compared to HET and WT controls (1-way ANOVA p=0.1133).

### Example 4

### Generation of a Bigenetic Model Overexpressing hAPP and hQC

(A) Crossbred and Breeding Performance of hQC-63 and hAPP-35

The vector maps of APP and hQC may be seen in FIG. 32. As described in Example 3(B)(ii) and (iii), breeding performance of APPwt-35 and hQC-63 mouse lines were excellent. Crossbreeding of APPwt-35 and hQC-63 yields the mouse line APP35/hQC63. The Genotypes for both transgene integrations APP35 and hQC63 were determined as described in Example 3(C)(ii) and (vi), respectively. Genotype groups of Hom/Het, Het/Het and Wt/Het animals were observed up to an age of 26 months.

### (B) ELISA

The Aβ accumulation in brain of transgenic animals was assessed applying ELISAs, which detect total Aβ42 (Aβₓ₋₄₂) and Aβ_{3(pE)-42}.

Mice of different age were sacrificed and the brain removed. The cerebellum was dissected from the residual brain, and the cerebrum was subjected to a sequential extraction of Aβ in 2% SDS and 70% formic acid. Brain tissues were homogenized in 2% SDS in distilled water (SDS fraction), sonicated and centrifuged at 75,500×g for 1 hour at 4° C. The supernatant was stored at −80° C. and the pellet suspended in 70% formic acid and neutralized using 1M Tris, pH 9.0 (formic acid fraction). The Aβ concentrations of the SDS and FA fractions were determined and the total Aβ burden calculated on the basis of the wet tissue weight. The ELISA was performed according to the manufacturer's protocol (IBL-Hamburg, Germany).

TABLE 5
Aβ and pE-Aβ ELISA at different ages of APP/hQC mice
APP35/hQC63 SDS fraction ng/mg brain

| | | | |
|---|---|---|---|
| Abeta x-42 | 12 months | 18 months | 2 Years |
| Hom/Het | nd | 68.1 | 6413.2 |
| HET/Het | 0 | 20.9 | 34.8 |
| Wt/Het | 0 | nd | 25.8 |
| pE-Abeta x-42 | 12 months | 18 months | 2 Years |
| Hom/Het | nd | 0.5 | 43.7 |
| HET/Het | 0 | 0 | 0.6 |
| Wt/Het | 0 | nd | 0.2 |
| | N = 3 | N > 5 | N > 5 |

nd = not determined

As depicted in Table 5, deposition of Aβ starts at 18 months age and significantly increases in animals >24 months in homozygous/heterozygous animals only. The increase of Aβ is combined with an increase of pE-Aβ too. The result correlates with separate histology results (not shown) where extra cellular plaques formation starts at 18 months of age in homozygous/heterozygous animals.

### (C) Histology

Histology was performed on paraffin brain sections of APP35/hQC63 mice to evaluate APP expression and amyloid pathology. These neuropathological changes were characterized in detail by immunohistochemistry (IHC) and histochemistry (CongoRed staining) on coronal sections of APP35/hQC63 brains (aged animals, 18, 24 & 26 months). The following antibodies were used:
N3pE-Aβ-specific antibody (rabbit polyclonal, 218003, Synaptic Systems, Göttingen, Germany; dilution 1:100000)
6E10 x-4x-Aβ-specific antibody (mouse monoclonal, Calbiochem, Merck, Darmstadt, Germany; dilution 1:30000)
APP C-terminal-specific antibody (rabbit polyclonal, 127003, Synaptic systems, 1:100000)
N1-4x-Aβ (N)-terminal-specific antibody (rabbit polyclonal, 18584, IBL, 1:30000)
N11pE Aβ-specific antibody (Probiodrug; mouse monoclonal, 1:30000)
Glia-specific antibody GFAP (rabbit polyclonal, Z0334, DAKOCytomation, Glostrup, Denmark; dilution 1:5000)

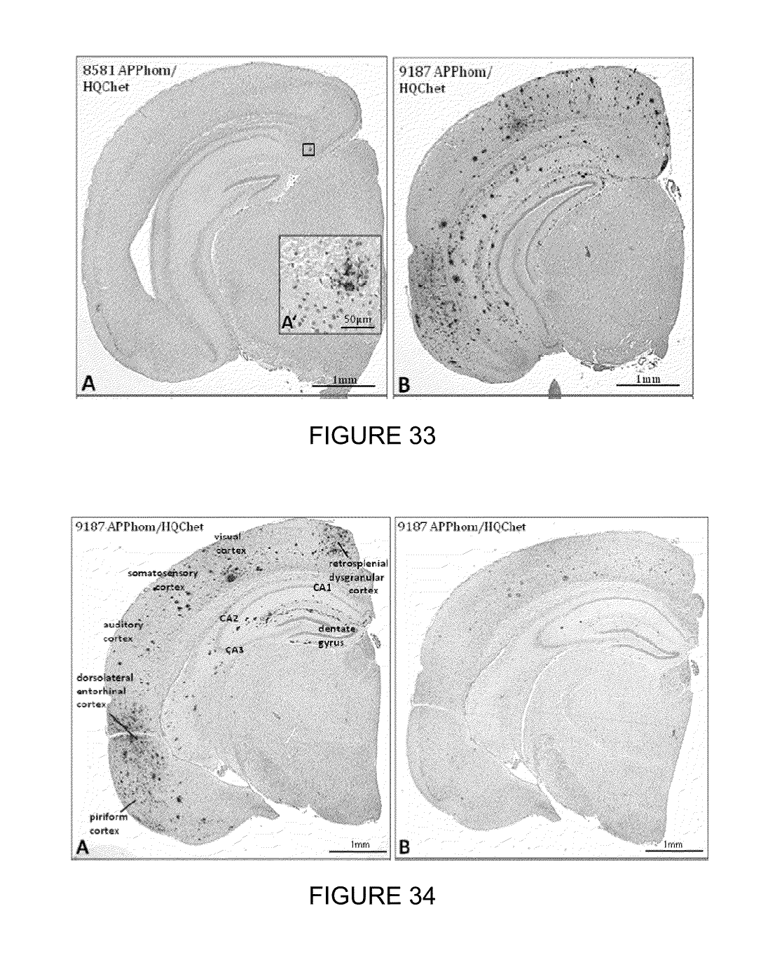

Animals (“APP35hom/hQC63het” or “APPhom/HQChet”) up to 18 months of age showed no evidence of amyloid plaques. First rare amyloid plaques appeared in animals at the age of 18 months (FIG. 33A), which was shown by IHC with an anti-N3pE-Aβ antibody.

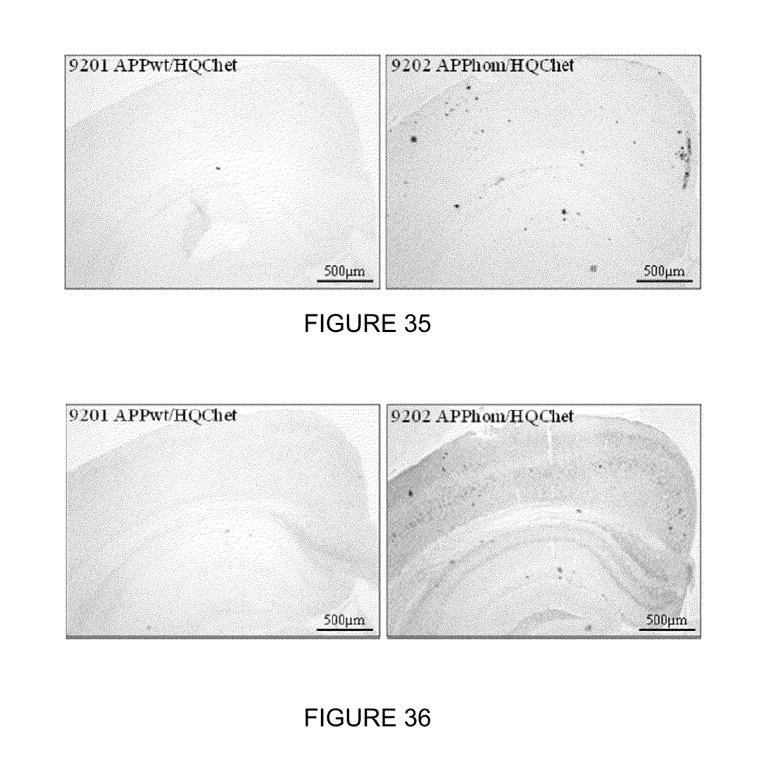

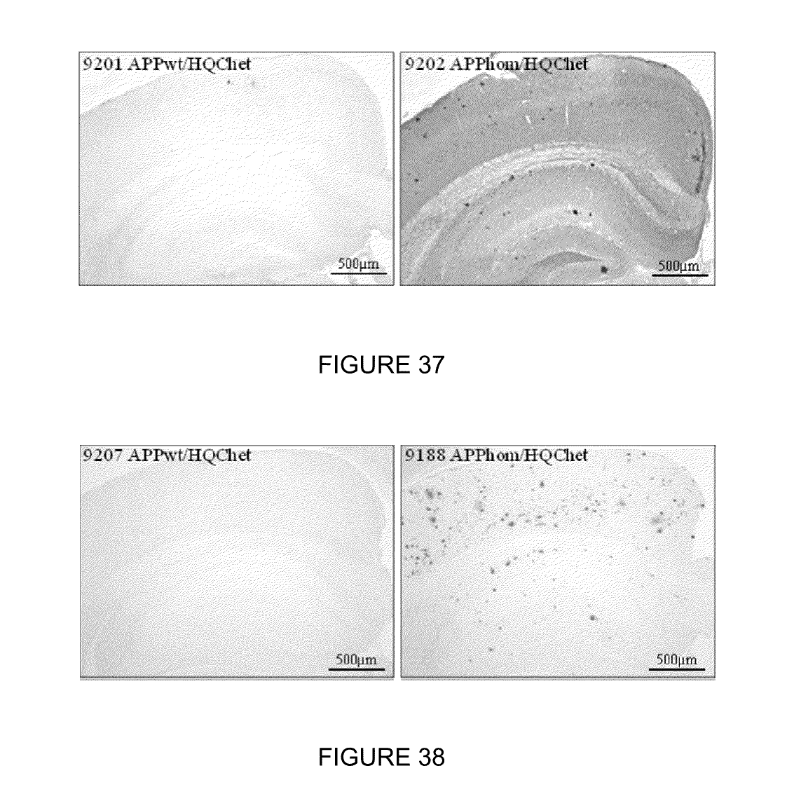

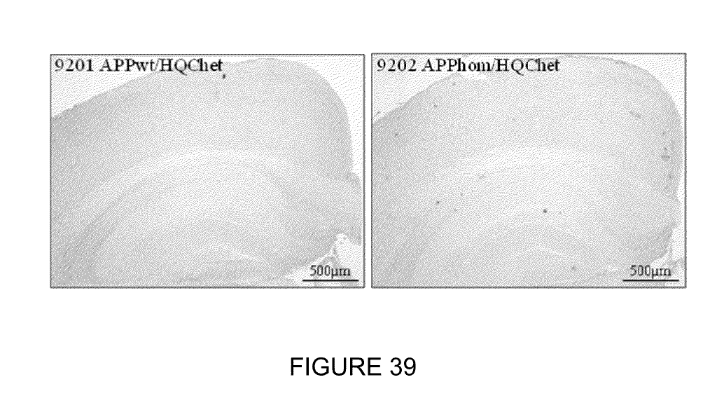

Later on (ages 24 & 26 months,) IHC reveals strong APP/Aβ-immunoreactivity in APPhom/HQChet animals compared to APP35/HQC63het within hippocampal and cortical areas (FIGS. 33-38). Plaques are reactive for anti-N3pE-Aβ (FIGS. 33B, 34A, 35), anti-APP (C-terminal) (FIGS. 34B, 36), anti-Aβ x-4x (6E10) (FIG. 37) and anti-Aβ N1-4x (FIG. 38).

The overexpression of APP is verified by intraneuronal and extracellular APP (in addition to the involvement in amyloid plaques), which is immunoreactive to both the APP C-terminal antibody and the 6E10 antibody (FIGS. 36 & 37). Both stainings reveal a prominent difference between APPwt/HQChet and APPhom/HQChet.

Aβ antibodies show higher immunoreactivity to amyloid plaques compared with antibodies, which are specific to the precursor protein. Therefore, in our characterization of the APP35/hQC63 line anti-N3pE-Aβ and anti-N1-4x-Aβ revealed the most specific staining of plaques and almost no staining of neurons and plaque-free areas (FIGS. 33B, 34A, 35 & 38).

N-terminally truncated Aβ species (N3pE-Aβ; N11pE-Aβ) were clearly detectable (FIGS. 33B, 34A, 35 & 39). The relative ratio of the different Aβ species could not be determined by means of IHC, because variable affinities of the antibodies preclude a valid comparison of their abundance.

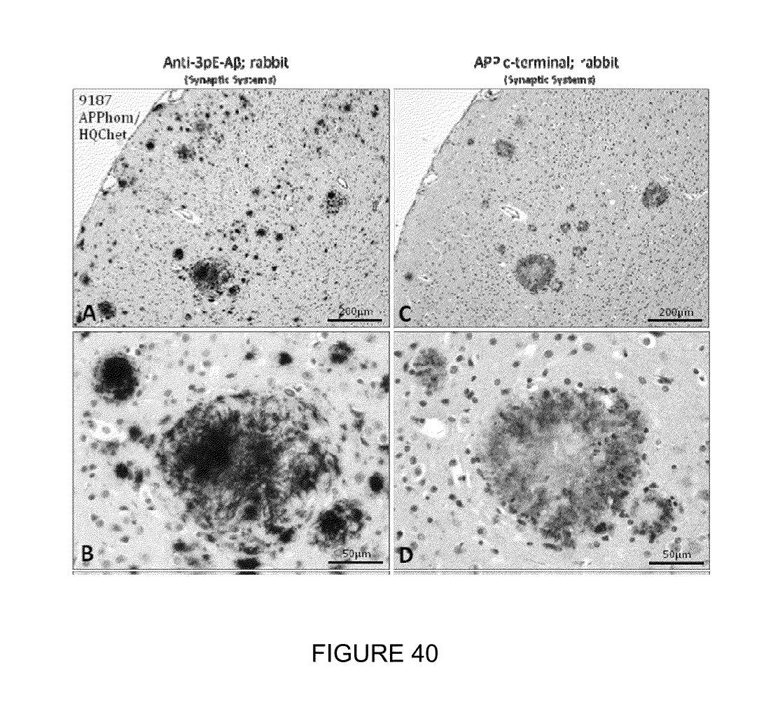

Amyloid plaques can be classified in two groups based on structural and morphological characteristics. Dense-core plaques are fibrillar deposits of Abeta with the classical properties of amyloid (beta-sheet secondary structure), while diffuse plaques exhibit a more amorphous character. FIG. 40 illustrates that anti-N3pE-Abeta is immunoreactive to dense core plaques and diffuse plaques (A, B) whereas anti-APP only binds to the dense core type (C, D).

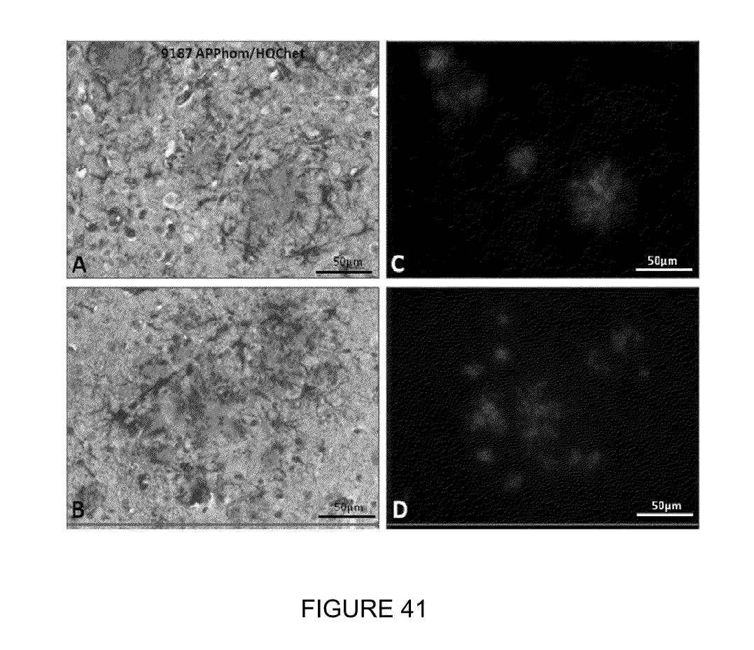

Dense core plaques are conventionally detected by Congo Red staining (FIG. 41) which shows a fluorescent activity when bound to amyloid fibrils with beta-sheet secondary structure. Therefore Congo Red serves as an additional marker for dense core plaques beside immunohistochemistry.

FIGS. 41A, B demonstrate, that numerous GFAP-positive activated glia cells are localized in the area of dense core plaques. This indicates that neuroinflammatory processes are running in plaque-affected regions of the brain.

### (D) Behavior APP/hQC

### Methods:

### Tail Suspension

The tail suspension test is the most widely used paradigm for the investigation of depressive behavior in rodents. Animals are suspended by the tail without chance to escape. An extended duration of immobility during a 6-minute trial indicates depressive behavior.

### Tail Flick and Pole Test

See Example 3(I)

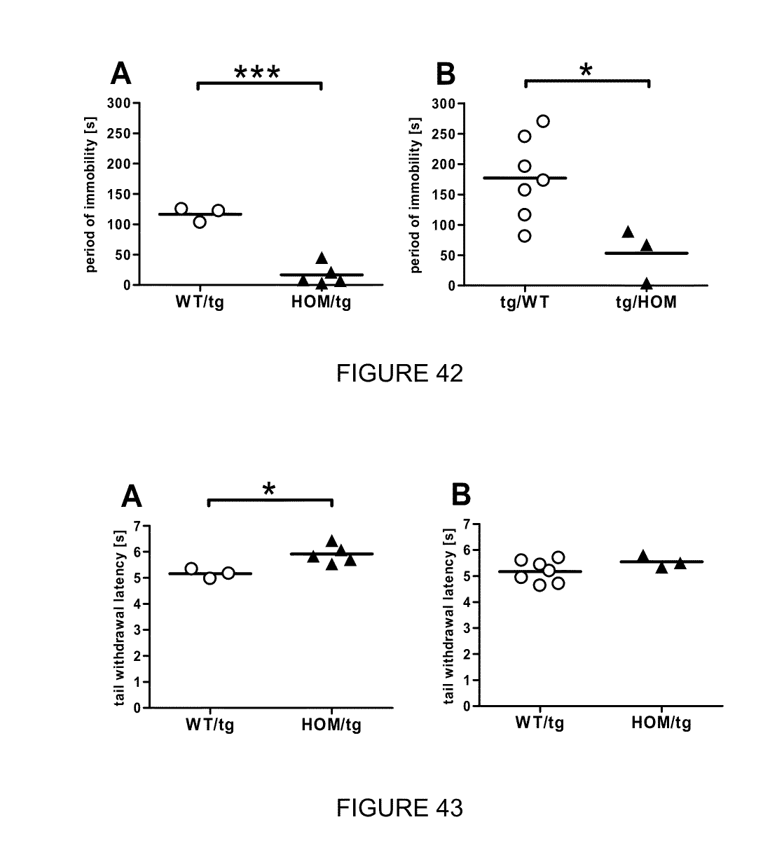

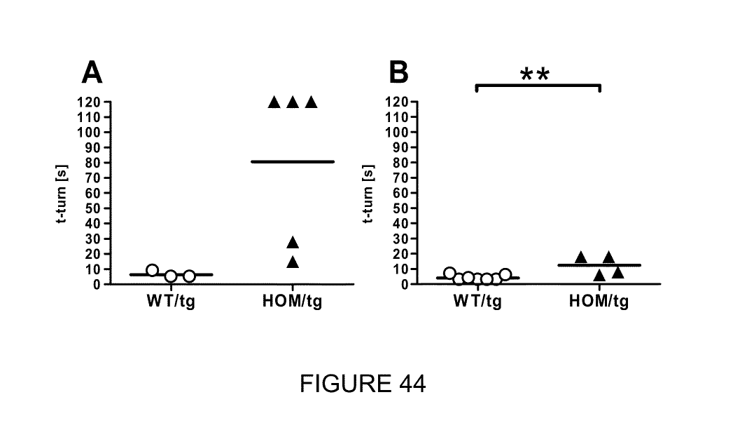

Pilot experiments (with up to 11 different behavioral tests) were conducted using the following two APP35/hQC63 female groups
set 1: PT_{—}02 at the age of 23-24 months (WT/tg n=3, HOM/tg n=4)
set 2: PT_{—}08 at the age of 25 months (WT/tg n=7, HOM/tg n=4)
   delivered clearly decreased periods of immobility in HOM/tg groups of both sets in the tail suspension test (FIG. 42). In addition HOM/tg females of set 1 were found to display increased tail withdrawal latencies in the tail flick assay (FIG. 43) as well as increased t-turn latencies in the pole test (FIG. 44) compared to WT/tg littermates.

The results presented herein provide the first indications for transgene-driven behavioral alterations in APP35/hQC63 mice.

What is claimed is:

1. A transgenic non-human animal for overexpressing amyloid precursor protein (APP) and glutaminyl cyclase (QC).

2. The transgenic non-human animal of claim 1, which overexpresses amyloid precursor protein (APP) and glutaminyl cyclase (QC).

3. The transgenic non-human animal of claim 1, which comprises cells containing one or more DNA transgenes encoding human APP and human QC.

4. The transgenic non-human animal of claim 3, wherein the human QC comprises the amino acid sequence of SEQ ID NO: 1 or an amino acid sequence having at least 75% sequence identity to the amino acid sequence of SEQ ID NO: 1 or a fragment or derivative of the amino acid sequence of SEQ ID NO: 1.

5. The transgenic non-human animal of claim 3, wherein the human QC consists of the amino acid sequence of SEQ ID NO: 1.

6. The transgenic non-human animal of claim 3, wherein the human APP comprises human APP695 or human APP770.

7. The transgenic non-human animal of claim 6, wherein the human APP comprises human APP695 as defined by the amino acid sequence of SEQ ID NO: 2 or an amino acid sequence having at least 75% sequence identity to the amino acid sequence of SEQ ID NO: 2 or a fragment or derivative of the amino acid sequence of SEQ ID NO: 2.

8. The transgenic non-human animal of claim 7, wherein the human APP695 consists of the amino acid sequence of SEQ ID NO: 2.

9. The transgenic non-human animal of any of claim 3, wherein the DNA transgene encoding QC comprises the nucleotide sequence of SEQ ID NO: 4 or substantially the same nucleotide sequence of SEQ ID NO: 4.

10. The transgenic non-human animal of claim 9, wherein the DNA transgene encoding QC consists of the nucleotide sequence of SEQ ID NO: 4.

11. The transgenic non-human animal of any of claim 3, wherein the DNA transgene encoding APP695 comprises the nucleotide sequence of SEQ ID NO: 5 or substantially the same nucleotide sequence of SEQ ID NO: 5.

12. The transgenic non-human animal of claim 11, wherein the DNA transgene encoding APP695 consists of the nucleotide sequence of SEQ ID NO: 5.

13. The transgenic non-human animal of claim 1, wherein the animal is heterozygous for at least one of the transgenes.

14. The transgenic non-human animal of claim 1, wherein the animal is homozygous for at least one of the transgenes.

15. The transgenic non-human animal of claim 1, wherein the animal is homozygous for the APP transgene and heterozygous for the QC transgene.

16. The transgenic non-human animal of any of claim 1, wherein the animal is a mouse.

17. The transgenic non-human animal according to claim 1, wherein each of the transgenes are operably linked to a tissue-specific promoter.

18. A method of producing a transgenic non-human animal for overexpressing APP and QC, wherein said method comprises crossbreeding a transgenic non-human animal comprising cells containing a DNA transgene encoding human APP with a transgenic non-human animal comprising cells containing a DNA transgene encoding human QC.

19. A transgenic non-human animal, which overexpresses APP and QC, obtainable by the method as defined in claim 18.

20. A method of screening for biologically active agents that inhibit or promote QC production in vivo, comprising:
administering a test agent to the transgenic non-human animal of claim 1; and
determining the effect of the agent on the amount of QC produced or on the QC activity.

21. A cell or cell line derived from the transgenic non-human animal according to claim 1.

22. A transgenic mouse comprising a transgenic nucleotide sequence encoding QC, which comprises the nucleotide sequence of SEQ ID NO: 4 or substantially the same nucleotide sequence of SEQ ID NO: 4, and a transgenic nucleotide sequence encoding APP, which comprises the nucleotide sequence of SEQ ID NOS: 5 or 6 or substantially the same nucleotide sequences of SEQ ID NOS: 5 or 6, operably linked to a promoter, integrated into the genome of the mouse, wherein the mouse demonstrates a phenotype that can be reversed or ameliorated with an QC inhibitor.

23. A method of screening for therapeutic agents that inhibit or promote QC activity comprising:
(a) administering test agents to the transgenic non-human animal as defined in claim 1;
(b) evaluating the effects of the test agent on the neurological phenotype of the transgenic non-human animal; and
(c) selecting a test agent which inhibits or promotes QC activity.

24. A method of investigation of the physiological function of QC comprising:
(a) Crossbreeding of the APP and QC transgenic non-human animals of claim 1 with a non-human animal model, which is specific for a desired disease,
(b) Breeding and ageing the crossbred animals and the disease specific animals;
(c) Monitoring the disease state age-dependently in the crossbred animals,
(d) As a control group, monitoring the disease state age-dependently in the disease specific animal models that are not transgenic for APP and QC,
(e) Calculating the differences in the disease state in the crossbred animals versus the disease specific animals, and
(f) Determining the effect of the APP and QC transgenes on the disease state.

25. The method of claim 24, comprising at least one feature selected from the group consisting of:
wherein the crossbred animals are heterozygous for the APP and QC transgenes;
wherein the crossbred animals are homozygous for the APP and QC transgenes;
wherein the crossbred animals are homozygous for the APP transgene and heterozygous for the QC transgene;
wherein the recombinant QC, which is overexpressed in the crossbred non-human animals, leads to one or more of the following effects on the disease state: an earlier outbreak of the specific disease, an accelerated course of the specific disease and/or a more severe course of the specific disease;
wherein the recombinant QC leads to the increase or decrease of the level of one or more QC substrates in the crossbred non-human animals;
wherein the disease specific animal model is selected from PDAPP, Tg2576, APP23, TgCRND8, PSEN1M146V or PSEN1M146L, PSAPP, APPDutch, BRI-Aβ40 and BRI-Aβ42, JNPL3, TauP301S, TauV337M, TauR406W, rTg4510, Htau, TAPP and 3×TgAD;
wherein the recombinant QC leads to the increase or decrease of the level of one or more QC substrates in the crossbred non-human animals and the QC substrate is selected from [Glu3]Aβ3-40/42/43 or [Glu11]Aβ11-40/42/43;
wherein the disease specific animal model is the apoE deficient mouse; and
wherein the recombinant QC leads to the increase or decrease of the level of one or more QC substrates in the crossbred non-human animals and the QC substrate is a chemokine selected from CCL2, CCL8, CCL7, CCL13, CCL 16, and CCL 18.

26. A method of screening for activity decreasing effectors of QC comprising:
(a) Crossbreeding of the APP and QC transgenic non-human animals of claim 1 with a non-human animal model, which is specific for a desired disease,
(b) Administering a test agent to a treatment group of crossbred animals,
(c) Administering a μlacebo to a control group of crossbred animals,
(d) Monitoring the disease state age-dependently in the treatment group,
(e) Monitoring the disease state age-dependently in the control group,
(f) Calculating the differences in the disease state in the treatment group versus the control group, and
(g) Determining the effect of the test agent on the disease state.

27. The method of claim 26, comprising at least one feature selected from the group consisting of:
wherein the crossbred animals are heterozygous for the APP and QC transgenes;
wherein the crossbred animals are homozygous for the APP and QC transgenes;
wherein the crossbred animals are homozygous for the APP transgene and heterozygous for the QC transgene;
wherein the recombinant QC, which is overexpressed in the crossbred non-human animals, leads to one or more of the following effects on the disease state: an earlier outbreak of the specific disease, an accelerated course of the specific disease and/or a more severe course of the specific disease;
wherein the recombinant QC leads to the increase or decrease of the level of one or more QC substrates in the crossbred non-human animals;
wherein the disease specific animal model is selected from PDAPP, Tg2576, APP23, TgCRND8, PSEN1M146V or PSEN1M146L, PSAPP, APPDutch, BRI-Aβ40 and BRI-Aβ42, JNPL3, TauP301S, TauV337M, TauR406W, rTg4510, Htau, TAPP and 3×TgAD.
wherein the recombinant QC leads to the increase or decrease of the level of one or more QC substrates in the crossbred non-human animals and the QC substrate is selected from [Glu3]Aβ3-40/42/43 or [Glu11]Aβ11-40/42/43;
wherein the disease specific animal model is the apoE deficient mouse; and
wherein the recombinant QC leads to the increase or decrease of the level of one or more QC substrates in the crossbred non-human animals and the QC substrate is a chemokine selected from CCL2, CCL8, CCL7, CCL13, CCL 16, and CCL 18.

28. A pharmaceutical composition comprising the selected test agent as defined in claim 23.

29. A method of treatment or prevention of a QC-related disease comprising:
(a) administering to a subject in need thereof the test agent as selected by the method of claim 23, wherein the QC-related disease is selected the group consisting of mild cognitive impairment, Alzheimer's disease, Familial British Dementia, Familial Danish Dementia, neurodegeneration in Down Syndrome, Huntington's disease, Kennedy's disease, ulcer disease, duodenal cancer with or w/o Helicobacter pylori infections, colorectal cancer, Zolliger-Ellison syndrome, gastric cancer with or without Helicobacter pylori infections, pathogenic psychotic conditions, schizophrenia, infertility, neoplasia, inflammatory host responses, cancer, malign metastasis, melanoma, psoriasis, rheumatoid arthritis, atherosclerosis, pancreatitis, restenosis, lung fibrosis, liver fibrosis, renal fibrosis, graft rejection, acquired immune deficiency syndrome, impaired humoral and cell-mediated immune responses, leukocyte adhesion and migration processes in the endothelium, impaired food intake, impaired sleep-wakefulness, impaired homeostatic regulation of energy metabolism, impaired autonomic function, impaired hormonal balance or impaired regulation of body fluids, multiple sclerosis, the Guillain-Barré syndrome and chronic inflammatory demyelinizing polyradiculoneuropathy; or
(b) (i) administering the selected test agent of claim 23; and (ii) monitoring the patient for a decreased clinical index for QC-related diseases.


SEQUENCE LISTING (49 sequences)

SEQ ID NO: 1 - protein, 361 aa, Human
MAGGRHRRVVGTLHLLLLVAALPWASRGVSPSASAWPEEKNYHQPAILNSSALRQIAEGTSISEMWQNDLQPLLIERYPGSPGSYAARQHIMQRIQRLQADWVLEIDTFLSQTPYGYRSFSNIISTLNPTAKRHLVLACHYDSKYFSHWNNRVFVGATDSAVPCAMMLELARALDKKLLSLKTVSDSKPDLSLQLIFFDGEEAFLHWSPQDSLYGSRHLAAKMASTPHPPGARGTSQLHGMDLLVLLDLIGAPNPTFPNFFPNSARWFERLQAIEHELHELGLLKDHSLEGRYFQNYSYGGVIQDDHIPFLRRGVPVLHLIPSPFPEVWHTMDDNEENLDESTIDNLNKILQVFVLEYLHL

SEQ ID NO: 2 - protein, 695 aa, Human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - protein, 770 aa, Human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 4 - DNA, 1086 bp, Human
atggcaggcggaagacaccggcgcgtcgtgggcaccctccacctgctgctgctggtggccgccctgccctgggcatccaggggggtcagtccgagtgcctcagcctggccagaggagaagaattaccaccagccagccattttgaattcatcggctcttcggcaaattgcagaaggcaccagtatctctgaaatgtggcaaaatgacttacagccattgctgatagagcgatacccgggatcccctggaagctatgctgctcgtcagcacatcatgcagcgaattcagaggcttcaggctgactgggtcttggaaatagacaccttcttgagtcagacaccctatgggtaccggtctttctcaaatatcatcagcaccctcaatcccactgctaaacgacatttggtcctcgcctgccactatgactccaagtatttttcccactggaacaacagagtgtttgtaggagccactgattcagccgtgccatgtgcaatgatgttggaacttgctcgtgccttagacaagaaactcctttccttaaagactgtttcagactccaagccagatttgtcactccagctgatcttctttgatggtgaagaggcttttcttcactggtctcctcaagattctctctatgggtctcgacacttagctgcaaagatggcatcgaccccgcacccacctggagcgagaggcaccagccaactgcatggcatggatttattggtcttattggatttgattggagctccaaacccaacgtttcccaatttttttccaaactcagccaggtggttcgaaagacttcaagcaattgaacatgaacttcatgaattgggtttgctcaaggatcactctttggaggggcggtatttccagaattacagttatggaggtgtgattcaggatgaccatattccatttttaagaagaggtgttccagttctgcatctgataccgtctcctttccctgaagtctggcacaccatggatgacaatgaagaaaatttggatgaatcaaccattgacaatctaaacaaaatcctacaagtctttgtgttggaatatcttcatttgtaa

SEQ ID NO: 5 - DNA, 2088 bp, Human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatcttgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 6 - DNA, 2313 bp, Human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 7 - DNA, 38 bp, Artificial
  Synthetic Primer
aataatctcgaggccaccatgctgcccggtttggcact

SEQ ID NO: 8 - DNA, 33 bp, Artificial
  Synthetic Primer
acatatgtacactagttctgcatctgctcaaag

SEQ ID NO: 9 - DNA, 23 bp, Artificial
  Synthetic Primer
aactcttggcacctagaggatct

SEQ ID NO: 10 - DNA, 23 bp, Artificial
  Synthetic Primer
tcctggtgtaagaatttgcactt

SEQ ID NO: 11 - DNA, 18 bp, Artificial
  Synthetic Primer
gctgtcaccccagaggag

SEQ ID NO: 12 - DNA, 20 bp, Artificial
  Synthetic Primer
cagaggaaggacctcgacct

SEQ ID NO: 13 - DNA, 27 bp, Artificial
  Synthetic Probe
cagcagaacggctacgaaaatccaacc

SEQ ID NO: 14 - DNA, 22 bp, Artificial
  Synthetic Primer
agcaagcctggaagacctggga

SEQ ID NO: 15 - DNA, 22 bp, Artificial
  Synthetic Primer
agactcagcccatccactcctt

SEQ ID NO: 16 - DNA, 38 bp, Artificial
  Synthetic Primer
aataatctcgaggccaccatggcaggcggaagacaccg

SEQ ID NO: 17 - DNA, 26 bp, Artificial
  Synthetic Primer
acatatgtacattacaaatgaagata

SEQ ID NO: 18 - DNA, 23 bp, Artificial
  Synthetic Primer
tcctacaagtctttgtgttggaa

SEQ ID NO: 19 - DNA, 19 bp, Artificial
  Synthetic Primer
gaaggacttggggagggag

SEQ ID NO: 20 - DNA, 27 bp, Artificial
  Synthetic Probe
caagtaagtcgaggtccttcctctgca

SEQ ID NO: 21 - DNA, 22 bp, Artificial
  Synthetic Primer
agcaagcctggaagacctggga

SEQ ID NO: 22 - DNA, 22 bp, Artificial
  Synthetic Primer
agactcagcccatccactcctt

SEQ ID NO: 23 - DNA, 23 bp, Artificial
  Synthetic Primer
aactcttggcacctagaggatct

SEQ ID NO: 24 - DNA, 23 bp, Artificial
  Synthetic Primer
tcctggtgtaagaatttgcactt

SEQ ID NO: 25 - DNA, 449 bp, Artificial
  Synthetic Fragment
aactcttggcacctagaggatctcgaggccaccatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccagga

SEQ ID NO: 26 - DNA, 20 bp, Artificial
  Synthetic Primer
tgcccatatgtcctaagctc

SEQ ID NO: 27 - DNA, 20 bp, Artificial
  Synthetic Primer
tggtcatggcatctgttcac

SEQ ID NO: 28 - DNA, 20 bp, Artificial
  Synthetic Primer
ggaacaagatcctgcgaatg

SEQ ID NO: 29 - DNA, 733 bp, Artificial
  Synthetic Fragment
ggaacaagatcctgcgaatgaatgagaagggagtcagatacctcagcagctagtagtcccagtgccagggcaggagcctcctccagggagccgcagacaccaacacatgtattccccgcttatgctcaggattctccccctgaagtatgtggagctgcaaatctatgaccggacgcagcgcatcctccgcgttaggacagtgacggagaagatctactacctgaggctccacgagaaacatccacaggctgtgtttcagttctggatccggttggtgaaaattttacagaaaggtctgtccatcaccaccaaagacccaagaatccagttcactcactgcctggtgcccaagatgtccaactcctccactgaaacagcagtaagtgccccccccccccccggcggtctgcgcagaaatgtatatatccatgtatatactcatacactcacatgcacgcactcgtatgttcccagatgcaaatggacacctagcctttcatagatgccggtcactcatccatagattcactcaacaccttggatcagaaatattcaggaaacaaacactgctcccttctcactgttccctgaacaatacagtgggatagtctatttacatagtattgtgttgtgtgaggcatgtgcaaaagctatgtcattgcaaataagggacttgattataggggaggtgtcttagttagggttttacttctgtgaacagatgccatgacca

SEQ ID NO: 30 - DNA, 506 bp, Artificial
  Synthetic Fragment
ggaacaagatcctgcgaatgaatgagaagggagtcagatacctcagcagctagtagtcccagtgccagggcaggagcctcctccagggagccgcagacaccaacacatgtattccccgcttatgctcaggattctccccctgaagtatgtggagctgcaaatctatgaccggacgcagcgcatcctccgcgttaggacagtgacggagaagatctactacctgaggctccacgagaaacatccacaggctgtgtttcagttctggatccggttggtgaaaattttacagaaaggtctgtccatcaccaccaaagacccaagaatccagttcactcactgcctggtgcccaagatgtccaactcctccactgaatcggtgcgggcctcttcgctattacgccaggatcaattctaggacttaaggtaggaaactaagtggctgaaggtagagagagaataaggacagtgaacgagtgggtgggtggggagcttaggacatatgggca

SEQ ID NO: 31 - DNA, 21 bp, Artificial
  Synthetic Primer
ggccagaggagaagaattacc

SEQ ID NO: 32 - DNA, 23 bp, Artificial
  Synthetic Primer
ttccaacacaaagacttgtagga

SEQ ID NO: 33 - DNA, 965 bp, Artificial
  Synthetic Fragment
ggccagaggagaagaattaccaccagccagccattttgaattcatcggctcttcggcaaattgcagaaggcaccagtatctctgaaatgtggcaaaatgacttacagccattgctgatagagcgatacccgggatcccctggaagctatgctgctcgtcagcacatcatgcagcgaattcagaggctccaggctgactgggtcttggaaatagacaccttcttgagtcagacaccctatgggtaccggtctttctcaaatatcatcagcaccctcaatcccactgctaaacgacatttggtcctcgcctgccactatgactccaagtatttttcccactggaacaacagagtgtttgtaggagccactgattcagccgtgccatgtgcaatgatgttggaacttgctcgtgccttagacaagaaactcctttccttaaagactgtttcagactccaagccagatttgtcactccagctgatcttctttgatggtgaagaggcttttcttcactggtctcctcaagattctctctatgggtctcgacacttagctgcaaagatggcatcgaccccgcacccacctggagcgagaggcaccagccaactgcatggcatggatttattggtcttattggatttgattggagctccaaacccaacgtttcccaatttttttccaaactcagccaggtggttcgaaagacttcaagcaattgaacatgaacttcatgaattgggtttgctcaaggatcactctttggaggggcggtatttccagaattacagttatggaggtgtgattcaggatgaccatattccatttttaagaagaggtgttccagttctgcatctgataccgtctcctttccctgaagtctggcacaccatggatgacaatgaagaaaatttggatgaatcaaccattgacaatctaaacaaaatcctacaagtctttgtgttggaa

SEQ ID NO: 34 - DNA, 20 bp, Artificial
  Synthetic Primer
tgcccatatgtcctaagctc

SEQ ID NO: 35 - DNA, 21 bp, Artificial
  Synthetic Primer
ggagattgtacgctaggaagg

SEQ ID NO: 36 - DNA, 20 bp, Artificial
  Synthetic Primer
agaaagcctttttggcagtt

SEQ ID NO: 37 - DNA, 517 bp, Artificial
  Synthetic Fragment
agaaagcctttttggcagttcagtgtgatgtgtactgttgcctgactacccatgttcatcttaatcctgcctgtttattgctgtctgagcaaaagcagaacgcatgcttctgcatttctgaattccaactgttctggaccaattttagaactcatccttttctttattcattaggacaaaccagcaactacttattccaggagaaactggtaacattgtgaggttgctaattcctcaacctgtcttacttggcacatatttcatatgatttgaggttggattagtgatttcaagggtagtgctgaaggtcagtatatacaatatgtctctggaaattgtatattaattttccatgtatgctgtacagttatttatcaaaatatctttatgtttaaaccttgatatctgaaaataagtgagatgatattcaagtggcagaaaaattgaagtttcttctacaaatatgaggagtttttctattttaaaattataacatccttcctagcgtacaatctcc

SEQ ID NO: 38 - DNA, 416 bp, Artificial
  Synthetic Fragment
tgcccatatgtcctaagctccccacccacccactcgttcactgtccttattctctctctaccttcagccacttagtttcctaccttaagtcctagaattgatcctggcgtaatagcgaagaggcccgcaccgatcaacctgtcttacttggcacatatttcatatgatttgaggttggattagtgatttcaagggtagtgctgaaggtcagtatatacaatatgtctctggaaattgtatattaattttccatgtatgctgtacagttatttatcaaaatatctttatgtttaaaccttgatatctgaaaataagtgagatgatattcaagtggcagaaaaattgaagtttcttctacaaatatgaggagtttttctattttaaaattataacatccttcctagcgtacaatctcc

SEQ ID NO: 39 - DNA, 20 bp, Artificial
  Synthetic Primer
actgaactcaggctgtcagg

SEQ ID NO: 40 - DNA, 20 bp, Artificial
  Synthetic Primer
caggaggaatctggtcaatg

SEQ ID NO: 41 - DNA, 20 bp, Artificial
  Synthetic Primer
agcagagaccaaggaggatt

SEQ ID NO: 42 - DNA, 552 bp, Artificial
  Synthetic Fragment
caggaggaatctggtcaatgctcccccagcaatggttaatgcccactcatccaaggatgagaaggtcatttgatcacctcagttaagtgtggccttattaaatttaattcaaagggggagaccataccgtgagaaagtatgcctttcacagcttcccactctaacaagccaaatggtcttgtgctaaggagcgggtcatcaccccctcctccctattccctttctggcacctgaggctataaaaagctaaattatagacccctcttccttatctcttcctgactcccaagacttctaaggacatgagttatgtgctgagcccagcctgactcccaaggctgttaaggaggattctacatcctggatatagattcagagtgcctcccgcctgcagtctgaggtcggccttcatgtccccagatgcccacttctttgttctttgttaattccccctcaacccctccctatttcccttgctgtgtgcttaaaacctggcgtttcagcctaataaactgagtgagaccttgacaggaatcctccttggtctctgct

SEQ ID NO: 43 - DNA, 419 bp, Artificial
  Synthetic Fragment
caggaggaatctggtcaatgctcccccagcaatggttaatgcccactcatccaaggatgagaaggtcatttgatcacctcagttaagtgtggccttattaaatttaattcaaagggggagaccataccgtgagaaagtatgcctttcacagcttcccactctaacaagccaaatggtcttgtgctaaggagcgggtcatcaccccctcctccctattccctttctggcacctgaggctataaaaagctaaattatagacccctcttccttatctcttcctgactcccaagatccccgggcgagctcgaattcagagaccgggaaccaaactagcctttaaaaaacataagtacaggagccagcaagatggctcagtgggtaaaggtgcctaccagcaagcctgacagcctgagttcagt

SEQ ID NO: 44 - DNA, 20 bp, Artificial
  Synthetic Primer
cagggactttggtgcataag

SEQ ID NO: 45 - DNA, 20 bp, Artificial
  Synthetic Primer
attgatcctggcgtaatagc

SEQ ID NO: 46 - DNA, 580 bp, Artificial
  Synthetic Fragment
cagggactttggtgcataagtatgtaccatgcccttttttcacagtcctagctctgcagaagtgcagcctgaaggcctgtctgctgagaggacatgccctggagccctgaaacaggcacagtgggaggaggaacggaggatgacaggcatcaggccctcagtccaaaagcaaccacttgagaatgggctggagtacgaaacatggggtcccgtccctggatccctcctcaaagagtaataagtaaaatataaacaggtaccccaggccgttctgggtttgggttgtaatgggatccatttgcagagaactattgagacagcccagccgtactgtgacaggcaatgtgggggaggaggttgaatcacttggtatttagcatgaatagaataattccctgaacatttttcttaaacatccatatctaaattaccaccactcgctcccagtcttcctgcctttgcgccagcctcctgtctggccatgcctgaagaaggctggagaagccacccacctcaggccatgacactgccagccacttggcaggtgcagccaaacctgagctattacgccaggatcaat

SEQ ID NO: 47 - DNA, 400 bp, Artificial
  Synthetic Fragment
acgcagcgcatcctccgcgttaggacagtgacggagaagatctactacctgaggctccacgagaaacatccacaggctgtgtttcagttctggatccggttggtgaaaattttacagaaaggtctgtccatcaccaccaaagacccaagaatccagttcactcactgcctggtgcccaagatgtccaactcctccactgaatcggtgcgggcctcttcgctattacgccaggatcaattctaggacttaaggtaggaaactaagtggctgaaggtagagagagaataaggacagtgaacgagtgggtgggtggggagcttaggacatatgggcaggagtcccaggtcttccaggcttgctgacttggccagagggacagatgggtgtcatggccagctgc

SEQ ID NO: 48 - DNA, 400 bp, Artificial
  Synthetic Fragment
gcagctggccatgacacccatctgtccctctggccaagtcagcaagcctggaagacctgggactcctgcccatatgtcctaagctccccacccacccactcgttcactgtccttattctctctctaccttcagccacttagtttcctaccttaagtcctagaattgatcctggcgtaatagcgaagaggcccgcaccgatcaacctgtcttacttggcacatatttcatatgatttgaggttggattagtgatttcaagggtagtgctgaaggtcagtatatacaatatgtctctggaaattgtatattaattttccatgtatgctgtacagttatttatcaaaatatctttatgtttaaaccttgatatctgaaaataagtgagatgatattcaagtgg

SEQ ID NO: 49 - DNA, 400 bp, Artificial
  Synthetic Fragment
gccttattaaatttaattcaaagggggagaccataccgtgagaaagtatgcctttcacagcttcccactctaacaagccaaatggtcttgtgctaaggagcgggtcatcaccccctcctccctattccctttctggcacctgaggctataaaaagctaaattatagacccctcttccttatctcttcctgactcccaagatccccgggcgagctcgaattcagagaccgggaaccaaactagcctttaaaaaacataagtacaggagccagcaagatggctcagtgggtaaaggtgcctaccagcaagcctgacagcctgagttcagtccccacgaactacgtggtaggagaggaccaaccaactctggaaatctgttctgcaaacacatgctcacacac